(19) United States
(12) Patent Application Publication
(10) Pub. No.: US 2004/0048377 A1
(43) Pub. Date: Mar. 11, 2004

(54) Methods and compositions involved in groucho-mediated differentiation

(76) Inventor: Johan Ericson, Stockholm (SE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 09/998,861

(22) Filed: Nov. 1, 2001

Related U.S. Application Data
(60) Provisional application No. 60/245,002, filed on Nov. 1, 2000.

(51) Int. Cl. A61K 38/17; C12N 15/85; C12N 5/08

(52) U.S. Cl. 435/455; 514/12; 435/368

(57) ABSTRACT

The present invention is directed to methods and compositions involved in modulating the fate of cellular differentiation. More specifically, the invention is directed to groucho-mediated differentiation, involving the interaction between a groucho-interacting protein, which recruits a Grouch corepressor.

### RELATED APPLICATIONS

This application claims priority from U.S. Ser. No. 60/245,002, filed Nov. 1, 2000. The contents of this applications are incorporated herein by reference in its entirety.

### FIELD OF THE INVENTION

The invention relates to the field of cellular differentiation, including methods for and compositions involved in the guided modulation of the fate of cellular differentiation.

### BACKGROUND OF THE INVENTION

One of the wonders of biology is that a fertilized egg gives rise to an embryo made up of multiple cell types that are not only chemically different, but also arranged in a specific, three-dimensional pattern. Cells that inherited the same genetic material from the egg diverge into a variety of cell types. Such chemical and architectural variety is possible because genes are switched on or off, and are expressed differently in diverse tissues. Differentiation is the process by which cells in a multicellular organism become specialized for these particular functions. Understanding the mechanisms which regulate and control this cellular differentation process can provide novel targets for therapeutic intervention in cellular disease. This cellular differentiation is especially relevant for the development of neurons, which are not only produced during the embryonic and early postnatal period but also continue to be generated in the adult brain from stem cells. More specifically, understanding the mechansisms which regulate differentiation of neuronal cells will lead to the development of therapies for the treatment of disorders of the central nervous system such as Parkinson's disease, Alzheimer's disease, stroke, obesity, diabetes, and spinal cord injury.

Pattern formation is the activity by which embryonic cells form ordered spatial arrangement of differentiated tissues. The physical complexity of higher organisms arises during embryogenesis through the interplay of cell-intrinsic lineage and cell-extrinsic signaling. Inductive interactions are essential to embryonic patterning in vertebrate develomment from the earliest establishment of the beody plan, to the patterning of the organ systems, to the generation of diverse cell types during tissue differentiation.

The patterning of many embryonic tissues depends on the secretion of inductive signals that specify distinct cell fates at different concentration thresholds, linking cell position to cell fate. The response of cells to graded inductive signals is often achieved through the patterned expression of transcription factors that serve as intermediaries in the position-dependent specification of cell fate. Insights into the principles of action of such transcriptional regulators have emerged from studies of embryonic patterning in Drosophila. In vertebrates, however, less is known about the strategies by which transcription factors control cell pattern in response to graded inductive signals. Understanding the role transcription factors play in cellular differentiation will provide valuable information leading to the provision of novel targets for intervention in cellular disorders and diseases.

One region of the vertebrate embryo in which graded inductive signals specify cell fates in a position-dependent manner is the neural tube. The generation of diverse neuronal subtypes within the vertebrate central nervous system (CNS) is an early and fundamental step in the assembly of neuronal circuits. Understanding the mechansisms which regulate the determination of neuronal fate is a key element in the development of therapies for the treatment of disorders of the central nervous system such as Parkinson's disease, Alzheimer's disease, stroke, obesity, diabetes, and spinal cord injury.

During the development of the vertebrate central nervous system, the assignment of regional identity to neural progenitor cells has a critical role in directing the subtype identity of post-mitotic neurons. In many regions of the CNS the specification of neuronal subtype identity is initiated by the imposition of distinct regional character on progenitor cells in the neural tube. Within the ventral half of the neural tube, the regional character of neuroepithelial cells is revealed through the spatially restricted expression of homeodomain transcription factors. Five distinct ventral progenitor domains can be identified in the combinatorial expression of a set of seven homeodomain proteins in response to the graded signalling activities of Sonic hedgehog (Shh) signalling in the ventral neural tube. These homeodomain proteins can be subdivided into class I and class II proteins, based on their repression or activation by Shh signals. Class I proteins comprise members of the Pax, Dbx and Irx families, and their expression is repressed by graded Shh signalling. Class II proteins comprise three Nkx proteins—Nkx6.1, Nkx2.2 and Nkx2.9 —and their expression in neural progenitors is dependent on Shh signalling. Specification of neuronal fate in the vertebrate CNS depends on the profile of transcription factor expression by neural progenitor cells. Characterizing the roles such transcription factors play in neurogenesis is a fey factor in understanding cellular differentiation, and is thus a key factor in treatments related to cellular differentiation.

The profile of class I and class II homeodomain protein expression within a progenitor cell appears to direct neuronal fate. The establishment of progenitor cell identity appears to involve cross-regulatory interactions between complementary pairs of class I and II HD proteins that share a common boundary. Class I and class II proteins that share a common progenitor cell boundary exhibit mutual cross-regulatory interactions. The complementary class I and class II protein pairs, Pax6 and Nkx2.2/Nkx2.9 and Dbx2 and Nkx6.1 mutually repress each others' expression. This regulatory network thus defines the boundaries of specific progenitor domains in response to the graded signalling activity of Shh. In addition, these cross-regulatory interactions help to maintain the identity of ventral progenitor cells over a prolonged period of neurogenesis, independent of ongoing Shh signalling.

As progenitors leave the cell division cycle and commit to a neuronal fate, the profile of homeodomain protein expression by ventral progenitor cells directs the subtype identity of post-mitotic neurons, thus generating motor neurons and interneurons at defined dorsoventral positions within the ventral neural tube. For example, Nkx6.1 is expressed in the three most ventral progenitor domains in the developing spinal cord that generate V2-neurons, motor neurons and V3 neurons. Nkx6.1 function in the pMN progenitor domain induces the expression of downstream neuronal subtype determinants such as MNR2 and Lim3, that direct motor neuron identity. Expression of the class I protein Irx3 within the p2 progenitor domain blocks the Nkx6.1-mediated induction of MNR2 but promotes Lim3 expression, resulting in the generation of V2 neurons. Similarly, Nkx2.2 expression in p3 domain progenitors prevents Nkx6.1 from initiating a motor neuron differentiation program and promotes the generation of V3 neurons.

At a mechanistic level, however, it remains unclear how cross-regulatory interactions between the class I and class II homeodomain proteins establish regional progenitor domains and control neuronal fate. The ability of class I progenitor homeodomain proteins to restrict the domain of expression of class II proteins, and vice versa, could reflect the function of these proteins as transcriptional activators, inducing the expression of downstream genes that function to repress target genes. Alternatively, class I and class II homeodomain proteins may functional primarily as transcriptional repressors, constraining the pattern of expression of target genes through their direct repressor activity. It is also possible that individual homeodomain proteins function both as activators and repressors, with distinct activities for different target genes. A critical issue in understanding cellular differentiation, which can lead to the provision of treatments for disorders related to cellular differentiation, is whether the patterning activities of progenitor HD proteins reflect their function as transcriptional activators or repressors.

A need remains to identify the molecular interactions mediated by class I and class II HD proteins which result in establishing progenitor cell identity and defining neuronal fate. Once these molecular interactions have been characterized, therapies can be provided which regulate the key components of these molecular interactions.

### SUMMARY OF THE INVENTION

The present invention identifies critical molecular interactions and complex formations involved in guiding the fate cellular differentiation. These complexes provide novel therapeutic targets for modulating guided cellular differentiation.

In one aspect, the invention involves a method of guiding the fate of differentiation of a cell into a specific cell type by providing a sample containing the cell and contacting the sample with a Groucho-interacting protein (GIP) in an amount and for a time sufficient to result in the formation of a complex between the GIP and a Groucho corepressor protein. In this aspect, the GIP and Groucho-corepressor protein complex represses DNA transcription and suppresses alternative pathways of differentiation in order to guide the fate of differentiation of the cell into a specific cell type.

In a further embodiment, this method also involves the step of contacting the cell with an exogenous Groucho corepressor protein. In a different embodiment, the Groucho corepressor protein is endogenous to the cell. Examples of suitable corepressor proteins include, but are not limited to, Grg1, Grg2, Grg3, and Grg4 and their human homologs.

In some embodiments, the GIP contains a TN-like domain. In other embodiments, the GIP is a homeodomain polypeptide. This homeodomain polypeptide may be a class II homeodomain polypeptide. This class II homeodomain polypeptide may be a member of the Nkx polypeptide family, such as Nkx2.2, Nkx2.9, Nkx6.1, Nkx6.2, and Nkx6.3 and their human homologs.

Using the methods of the claimed invention, the guided differentiation may result in the cell being differentiated into a motor neuron cell.

In other embodiments, the homeodomain polypeptide may be a class I homeodomain polypeptide. This class I homeodomain polypeptide may be selected from the group consisting of members of the Pax, Dbx, and Irx polypeptide families.

In some embodiments, the cell selected is a stem cell, such as a neural stem cell. In other embodiments, the cell is a progenitor cell.

The cells used in the invention may differentiate into a neuron, including an interneuron, a motor neuron, and a projection neuron. The projection neurons may be a dopaminergic neuron, a cortical neuron, a gaba-ergic neuron, or a glutaminergic neuron. In other embodiments, the cells used in the invention may differentiate into a stem cell, a cell of the peripheral nervous system, a kidney cell, a heart muscle cell, a pancreatic cell, a skin cell, a liver cell, and a white or red blood cell.

In some embodiments, the GIP is selected from the group consisting of Nkx6.1, Nkx6.2, Nkx6.3 and the cell type into which the cell differentiates is a beta cell producing insulin. In other embodiments, the GIP Nkx2.2 and the cell type into which the cell differentiates is a glucagon producing cell.

In various embodiments, the contacting of the sample with a GIP occurs either in vitro, ex vivo, or in vivo. In other embodiments, the GIP is a polypeptide having an amino acid sequence selected from the group consisting of SEQ ID NO: 7 and 13. In still other embodiments, the GIP is a polypeptide having the amino acid sequence X_{AA1}-X_{AA2}-X_{AA3}-X_{AA4}-X_{AA5}-X_{AA6}-X_{AA7}-X_{AA8}-X_{AA9}-X_{AA10}-X_{AA11}, wherein X_{AA1} is Thr, Leu, or Ser; X_{AA2} is Gly or Pro; X_{AA3} is Phe or His; X_{AA4} is Ser, Thr, Gly, or His; X_{AA5} is Val or Ile; X_{AA6} is Lys, Arg, Asn, or Ser; X_{AA7} is Asp or Ser; X_{AA8} is Is1 or Leu; X_{AA9} is Leu; X_{AA10} is Asp, Asn, Ser, or Gly; and X_{AA11} is Leu or Arg.

In another aspect, the invention includes an isolated polypeptide having an amino acid sequence selected from the group consisting of an amino acid sequence of SEQ ID NO: 7 or 13a variant of an amino acid sequence of SEQ ID NO: 7 or 13, wherein one or more amino acid residues in the variant differs from the amino acid sequence of the mature form, provided that the variant differs in no more than 15% of amino acid residues from this amino acid sequence.

In some embodiments, this polypeptide has the amino acid sequence of a naturally-occurring allelic variant of an amino acid sequence of SEQ ID NO: 7 or 13. This allelic variant may have an amino acid sequence that is the translation of a nucleic acid sequence differing by a single nucleotide from the nucleic acid sequence of SEQ ID NO: 12. This variant may also have a conservative amino acid substitution.

In yet another aspect, the invention involves an isolated nucleic acid molecule having a nucleic acid sequence encoding a polypeptide having an amino acid sequence, wherein the amino acid sequence encoded by the nucleic acid molecule is selected from the group consisting of the amino acid sequence of SEQ ID NO: 7 or 13 a variant of an amino acid sequence of SEQ ID NO: 7 or 13, wherein one or more amino acid residues in the variant differs from the amino acid sequence of the mature form, provided that the variant differs in no more than 15% of amino acid residues from the amino acid sequence; and wherein the nucleic acid molecule is selected from the group consisting of a nucleic acid fragment encoding at least a portion of a polypeptide having an amino acid sequence of SEQ ID NO: 7 or 13, or a variant of the polypeptide, wherein one or more amino acid residues in the variant differs from the amino acid sequence of the mature form, provided that the variant differs in no more than 15% of amino acid residues from the amino acid sequence; and a nucleic acid molecule containing the complement of any of these.

In some embodiments, the invention involves a vector containing the nucleic acid molecule of the invention. This vector may also contain a promoter operably-linked to the nucleic acid molecule. Also included in the invention is a cell containing this vector.

In another embodiment, the invention involves an antibody that binds immunospecifically to the polypeptide of the invention. The antibody may be a monoclonal antibody or a humanized antibody.

In a further aspect, the invention involves a peptide less than 400 amino acids in length that includes the amino acid sequence X_{AA1}-X_{AA2}-X_{AA3}-X_{AA4}-X_{AA5}-X_{AA6}-X_{AA7}-X_{AA8}-X_{AA9}-X_{AA10}-X_{AA11}, wherein X_{AA1} is Thr, Leu, or Ser; X_{AA2} is Gly or Pro; X_{AA3} is Phe or His; X_{AA4} is Ser, Thr, Gly, or His; X_{AA5} is Val or lie; X_{AA6} is Lys, Arg, Asn, or Ser; X_{AA7} is Asp or Ser; X_{AA8} is lie or Leu; X_{AA9} is Leu; X_{AA10} is Asp, Asn, Ser, or Gly; and X_{AA11} is Leu or Arg.

In another aspect, the invention involves a purified complex containing a first polypeptide and a second polypeptide, wherein the first polypeptide is a GIP and the second protein is a Groucho corepressor protein. In various embodiments, the first polypeptide is labeled or the second polypeptide is labeled. In some embodiments, the first polypeptide is selected from the group consisting of class I and class II homeodomain polypeptides. In other embodiments, the first polypeptide contains a TN domain. In other embodiments, the Groucho corepressor protein is selected from the group consisting of Grg1, Grg2, Grg3, or Grg4 and their mammalian homologs. In still other embodiments, the GIP contains a TN-like domain. In other embodiments, the GIP is a homeodomain polypeptide.

In a further aspect, the invention involves a purified complex containing a first polypeptide and a second polypeptide, wherein the first polypeptide contains a region of amino acids of a GIP sufficient to allow the first polypeptide to bind the second polypeptide, and wherein the second polypeptide has a region of amino acids of a Groucho corepressor protein sufficient to bind the first polypeptide.

In still a further aspect, the invention includes a chimeric polypeptide having a first domain covalently linked to a second domain, wherein the first domain contains six or more amino acids of the first polypeptide and the second domain contains six or more amino acids of the second polypeptide. This first domain may contain a GIP binding domain and the second domain may contain a Groucho corepressor binding domain. In one embodiment, the invention involves a nucleic acid encoding the chimeric polypeptide of the invention. The invention also includes a vector containing this nucleic acid as well as a cell containing this vector.

In various other embodiments, the invention involves an antibody, which specifically binds the complex of the invention. In another embodiment, the invention involves a kit containing a reagent, which can specifically detect the complex of the invention. This reagent may include an antibody specific for the complex, an antibody specific for the first polypeptide, and an antibody specific for the second polypeptide.

In yet another aspect, the invention involves a method of identifying an agent which modulates the stability or activity of the complex of the invention. This method involves the steps of providing the complex; contacting the complex with a test agent; and detecting whether the test agent modulates the stability or activity of the complex.

In a further aspect, the invention involves a method of identifying an agent which disrupts a polypeptide complex involving the steps of providing the complex of the invention; contacting the complex with a test agent; and detecting the presence of a polypeptide displaced from the complex, wherein the presence of displaced polypeptide indicates the agent disrupts the complex.

In still a further aspect the invention provides a method for the screening of a candidate substance interacting with the complex of the invention by providing the complex; obtaining a candidate substance; bringing into contact the complex with the candidate substance; and detecting the complexes formed between the polypeptide and the candidate substance.

Also provided is a method for the screening of a candidate substance interacting with a Groucho-corepressor protein, by providing a Groucho-corepressor protein; obtaining a candidate substance; bringing into contact the polypeptide with the candidate substance; and detecting the complexes formed between the polypeptide and the candidate substance.

The invention also involves a method for inhibiting the guided differentiation of a cell resulting in the impairment of ventral patterning by contacting the complex of the invention with an agent that disrupts the complex, thereby inhibiting the guided differentiation by disrupting the complex, which is necessary for differentiation.

In a further aspect, the invention involves a method of identifying a polypeptide complex in a subject by providing a biological sample from a subject and detecting, if present, the polypeptide complex of the invention in the sample, thereby identifying the complex.

In yet another aspect, the invention involves a method of determining altered expression of a polypeptide in a subject by providing a biological sample from the subject; measuring the level of the complex of the invention in the sample; and comparing the level of the complex from to the level of the complex in a reference sample whose level of the complex of the invention is known; thereby determining whether the subject has altered expression of the polypeptide.

Finally, the invention involves a method of treating or preventing a disease or disorder involving altered levels of the complex of the invention by administering a therapeutically effective amount of at least one molecule that modulates the function of the complex to a subject in need thereof.

### BRIEF DESCRIPTION OF THE FIGURES

FIG. 1: Class II Nkx proteins act as TN-domain-dependent repressors and interact with Gro/TLE Proteins in vitro. FIG. 1A shows an alignment of proteins with a TN (white band) and a HD (dark gray) domain. Conserved amino acids of the Nkx consensus are depicted by filled circles (>90%) or diamonds (70-90%). FIG 1B depicts a gel of a GST-Gro/Nkx binding assay. FIG 1C shows a gel indicating the binding of Nkx proteins (± the TN domain) with mouse Grg4. FIG. 1D shows EMSA assays of Nkx proteins. FIG. 1E presents a bar graph depicting TN domain-dependent repression of Grg4.

FIG. 2: Nkx proteins act as TN domain-dependent repressors in neural patterning in vivo. FIG. 2A depicts a graphic view of the Nkx constructs. FIG. 2B presents a graph of the activity of GAL4-Nkx constructs in a transcription reporter assay in COS-7 cells. FIG. 2C contains photographs depicting expression of Nkx and Pax proteins in the spinal cord of normal HH stage 20 chick embryos. FIG. 2D contains photographs depicting expression of Nkx, Dbx and Pax proteins in the ventral spinal cord of normal HH stage 20 chick embryos.

FIG. 3 shows figures depicting ectopic expression of Nkx proteins. FIG. 3A shows the consequences of misexpression of Nkx proteins on Sim1 expression. FIG. 3B shows the consequences of misexpression of Nkx proteins on MN marker expression. FIG. 3C shows the consequences of misexpression of Nkx proteins on MN (MNR2 and Hb9) and interneuron marker expression.

FIG. 4 shows classI proteins interacting with Gro/TLE proteins and acting as eh1 domain-dependent repressors in neural patterning in vivo. FIG. 4A shows an alignment of class 1 proteins with the Nkx consensus domain and the eh1 domain. FIGS. 4B-4E show the interaction of GST-Gro fusion protein with class 1 proteins. FIGS. 4F-4I show the consequences of ectopic expression of Dbx on Nkx expression. FIGS. 4J-4M show the consequences of misexpression of Pax6 on p3 domain progenitors.

FIG. 5 depicts labelling experiments showing expression of Grg4 in the developing chick spinal cord.

FIG. 6A shows the interaction of GST-Gro with Grg5; FIG. 6B shows a bar graph depicting the effect of Grg5 on Nkx-mediated expression in vitro; FIG. 6C shows the consequences of ectopic expression of Grg5 on marker gene expression in the chick neural tube; FIG. 6D shows the consequences of ectopic expression of Nkx and Grg on Lim3 expression in chick neural tube.

FIG. 7 depicts a schematic version of a derepression model of ventral cell fate specification.

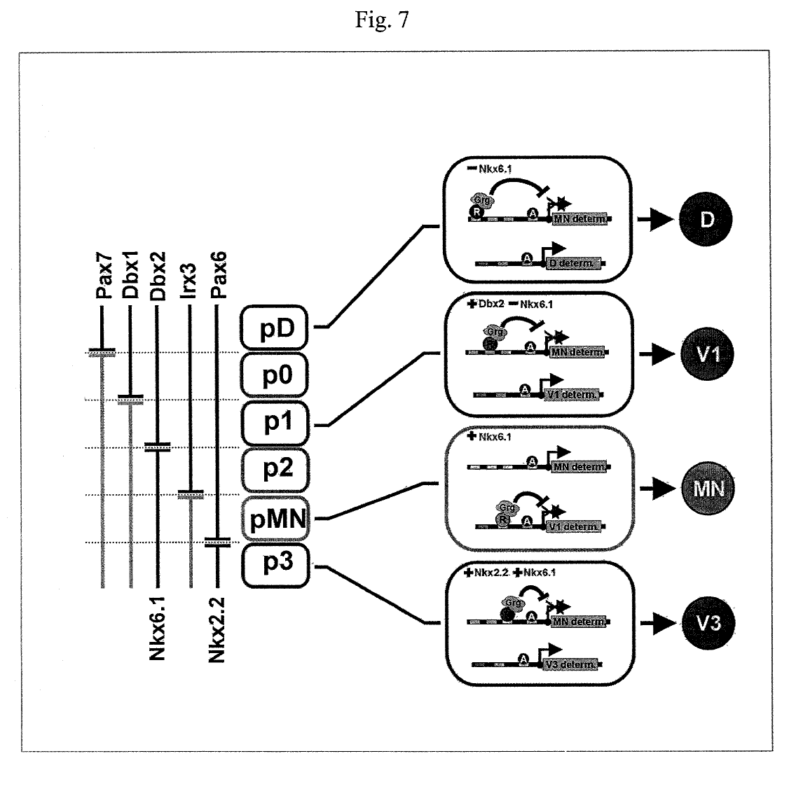

### DETAILED DESCRIPTION

This invention relates to the mechanisms underlying the cross-repressive interactions that occur between classes of transcription factor proteins, which establish progenitor domain identity and determine the fate of cellular differentiation of a variety of cell types. The identification of the molecular interactions involved in establishing progenitor cell identity and defining neuronal fate is a key component in providing therapies targeted to treatment of disorders related to cellular differentiation and to disorders of the CNS. Once these molecular interactions have been characterized, therapies can be provided which regulate the key components of these molecular interactions. The present invention demonstrates that transcription factors modulate the fate of differentiation of cells by functioning directly as transcriptional repressors, through the recruitment of corepressors, including the Gro/TLE class of corepressors. The Gro/TLE class of corepressors are referred to herein as Gro/TLE, Groucho, or Groucho-like corepressors, with all terms being used equivalently. Progenitor cells are then directed to individual neuronal fates by the suppression of alternative pathways of differentiation. The spatial pattern of neurogenesis has been shown to be achieved through the repression of repressors, a derepression strategy of neuronal determination.

In one aspect, the invention provides a method for guiding the fate of the differentiation of a cell, by providing to cells Groucho-like corepressor polypeptides and Groucho-interacting polypeptides (hereafter referred to as “GIP”) in amounts sufficient for the polypeptides to form a complex, wherein the complex suppresses alternative pathways of differentiation by causing repression of DNA transcription, thus resulting in the guided differentiation of the fate of a cell.

In another aspect, the invention is directed to compositions involved in the molecular interactions necessary to guide the fate of cellular differentiation. More specifically, these compositions include a functional domain that mediates neural patterning activity, a novel GIP named Nkx6.3, and novel complexes formed between a class of transcriptional repressors and a Groucho-like corepressor, which they recruit. The functional domain is the TN (NK decapeptide) domain. A class of such domains is provided herein. The invention also provides a novel consensus sequence for this domain, which is involved with the recruitment of a groucho-like corepressor. The consensus sequence is provided herein as SEQ ID NO: 7.

Another composition of the present invention is a novel GIP which helps guide the fate of cellular differentiation by recruiting a Groucho-like corepressor. The novel GIP is termed Nkx6.3 and is provided herein as SEQ ID NO: 12 (nucleic acid) and 13 (amino acid).

Yet another aspect of the present invention is the complex formed between the GIP and the groucho-corepressor proteins, with such complex playing a critical role in guiding the fate of cells in cellular differentiation.

Other aspects of the invention are directed to methods of screening for agents which modulate the stability or activity of the above complex. Such agents can play a role in guiding the fate of a cell in cellular differentiation through their role in either enhancing or inhibiting the critical role played by GIP-Groucho complex in cellular differentiation. Such screening methods include methods for identifying an agent which modulates the stability or activity of the complex, methods of identifying an agent which disrupts the polypeptide complex, methods for screening for candidate substances which interact with the complex, methods for identifying the polypeptide complex in a subject, and methods of determining altered expression of a polypeptide complex or component in a sample. Other methods of the present invention include methods of treating or preventing a disease or disorder involving altered levels of the complex by administering therapeutically effective levels of compositions which modulate the function of the complex.

Each of these aspects of the invention is described in more detail below.

Methods for Guiding the Fate of Cellular Differentiation

Evidence that the Nkx class homeodomain proteins and the Dbx class proteins pattern the neural tube through groucho-mediated repressive interactions is provided. It has been demonstrated that all class II proteins and most class I proteins are transcriptional repressors, and that the activity of these proteins relies on their ability to recruit a common class of corepressors, the Gro/TLE proteins. These findings indicate that Gro/TLE-mediated repression has a pivotal role in the control of ventral neural patterning. They also reveal that the patterning of the neural tube is achieved in large part by the spatially restricted repression of transcriptional repressors—thus invoking a derepression model of neuronal fate specification.

Graded Shh signaling appears to initiate the patterning in the ventral spinal cord by regulating the expression of class I and class II homeodomain proteins in neural progenitor cells. Individual class I and class II proteins appear to establish progenitor cell identity through their ability to restrict the expression of other homeodomain proteins to discrete domains of the ventral neural tube. The biochemical basis of neural patterning by class I and class II homeodomain proteins, however, remains unclear. This study provides evidence that most class I and class II homeodomain establish progenitor cell identity through their action as transcriptional repressors. Their repressor activity maps to a conserved eh1-like protein motif that appears to recruit a common class of Gro/TLE corepressor proteins. These findings suggest a model in which the specification of progenitor cell identity in the ventral neural tube is achieved by a common program of Gro/TLE mediated repression of homeodomain protein expression. An implication of this model is that the fate and pattern of neurogenesis in the ventral spinal cord, emerges through the derepression of genes that establishes the identity of differentiating neurons.

Groucho-Interacting Polypeptides (GIPs) as eh1 Domain-Dependent Transcriptional Repressors

A common molecular mechanism for the activities of class I and class II GIP proteins in the control of neural pattern has been demonstrated. Most class I and class II proteins influence neural pattern solely through their activity as repressors. A conserved eh1 domain (Smith and Jaynes, 1996) is found in all class I and class II proteins that function in the neural tube as repressors. There is a precise correlation between the presence of the eh1 motif in class I and class II proteins, their ability to bind Gro/TLE proteins in vitro, and their activity as repressors in vitro and in neural patterning in vivo. Thus, the patterning activities of the class II and class I proteins that possess an eh1 motif appear to be mediated exclusively through their ability to recruit a common class of Gro/TLE corepressors.

Hybrid class I and class II proteins consisting of the homeodomain fused to an EnR domain mimic the neural patterning activity of the corresponding full length proteins. In contrast, the homeodomain, when expressed alone or linked to a strong transcriptional activation domain, has no influence on neural cell pattern. These findings argue that regions other than the homeodomain contribute little to their specificity of action in neural patterning. The distinct functions of class I and class II proteins are therefore likely to be mediated primarily by the specificity of DNA binding inherent in the homeodomain. In support of this idea, Nkx2.2 and Nkx2.9 have the same patterning activities in the neural tube, highly conserved homeodomains (Pabst et al., 1998), and recognize the same target DNA sequence. Similarly, Nkx6.1 and Nkx6.2 have equivalent patterning activities in the neural tube (A. Vallstedt et al., in preparation), conserved homeodomains (Qui et al., 1998), and recognize the same DNA target sequence.

Groucho-Like Corepressors Play a Role in Guiding Cellular Differentiation

Several lines of evidence support a central role for Gro/TLE corepressors in neural patterning in vivo. First, the eh1 domain in class I and class II proteins predicts their repressor function of in vivo. Second, two Gro/TLE genes, Grg3 and Grg4, are expressed by ventral progenitor cells at the time that neural pattern is established. Third, overexpression of Grg5, a protein that inhibits Gro/TLE repressor function (Roose et al., 1998; Ren et al., 1999), results in a marked deregulation in the spatial pattern of class II proteins along the dorsoventral axis of the neural tube. Fourth, Grg5 expression blocks the inductive activity of Nkx6.1 in vivo.

The finding that overexpression of Grg5 elicits a dorsal expansion in the domains of expression of both class II proteins, Nkx6.1 and Nkx2.2, provides indirect evidence that Gro/TLE function is required normally to establish the p1/p2 and pMN/p3 progenitor domain boundaries. However, the dorsal expansion in the domains of expression of Nkx6.1 and Nkx2.2, was not accompanied by a complementary ventral expansion in the domains of expression of the class I proteins Dbx2 and Pax6. This asymmetry raises the possibility that a higher level of Gro/TLE protein is required to elicit the repressor activity of class I proteins than of class II proteins. Overexpression of Grg5 also disrupted the normal complementarity in patterns of expression of the class I/class II protein pairs Dbx2/Nkx6.1 and Pax6/Nkx2.2. Thus, in the presence of Grg5, regions of the neural tube in which Nkx6.1 was expanded dorsally coexpressed Dbx2. In addition, individual cells that expressed ectopic Nkx2.2 coexpressed Pax6. These findings imply that the presence of Grg5 reduces Gro/TLE activity below a level at which class II proteins are able to repress the expression of class I proteins.

Although our analysis has focused on the interaction between pairs of class I and class II proteins that share a common progenitor domain boundary, it is likely that Gro/TLE protein function is also necessary to delineate the expression of class I proteins for which complementary class II proteins have not yet been identified. In support of this idea, the expression patterns of the class I proteins Dbx1 and Pax7 are also altered by Grg5 overexpression.

In contrast to most class I proteins, however, Pax6 lacks an eh1 domain and appears to function in vivo as an activator rather than a repressor. Nevertheless, Pax6 is necessary and sufficient to repress Nkx2.2 expression in vivo, implying that Pax6 acts indirectly to induce an intermediary repressor protein. The finding that the domain of Nkx2.2 expands dorsally upon Grg5 overexpression therefore implies that the repressor protein activated by Pax6 itself functions in a Grg/TLE-dependent manner. Taken together, these observations suggest that the establishment and maintenance of many and perhaps all ventral progenitor domains, whether achieved by direct or indirect repression, depend on the activity of Gro/TLE corepressors.

GIPs Recruit Groucho-Like Corepressors, Leading to Cellular Differentiation by Derepression

Class II proteins that contain eh1 motifs also possesses transcriptional activation domains and, in certain contexts these proteins have been shown to function as transcriptional activators. Nevertheless, our results suggest that the activity of class II proteins in ventral neuronal cell fate specification can be attributed solely to their repressor activity. How do the Gro/TLE dependent repressor function of class II proteins specify distinct neuronal fates? We examine this issue through a description of the patterning activities of Nkx6.1. Our studies reveal that the role of Nkx6.1 in promoting motor neuron generation is mediated solely by its repressor activity. Thus, Nkx6.1 is likely to act within the pMN domain by excluding the expression of class I homeodomain proteins that have the capacity to suppress motor neuron differentiation. One relevant ventral repressor of motor neuron specification appears to be Dbx2. However, the repressor activity of Nkx6.1 is sufficient to specify motor neuron fate in more dorsal regions of the neural tube where progenitors do not express Dbx2. This finding implies that Nkx6.1 is able to repress the expression of suppressors of motor neuron specification along the entire dorsoventral axis of the neural tube. The identity of such dorsal Nkx6.1-sensitive repressors is not established, but may include the Gsh1 and Gsh2 homeodomain proteins since the expression of both proteins is normally restricted to the dorsal neural tube, but are ectopically activated within the ventral neural tube in Nkx6.1 mutants (Sander et al., 2000). Essentially, similar arguments can be made for the role of Nkx6.1 in promoting V2 neuronal fate within the p2 progenitor domain (Briscoe et al., 2000; Sander et al., 2000).

The role of Nkx6.1 repressor activity in promoting motor neuron fate and V2 fates in the p2 domain is also accompanied by a role of Nkx6.1 in suppressing the specification of V1 neurons within the pMN and p2 domains. V1 neurons are normally generated from Dbx2⁺ progenitors located within the p1 domain, and their exclusion from more ventral regions is dependent on Nkx6.1 activity (Sander at al., 2000). Our results show that the suppression of V1 fate can also be accounted for by the repressor activity of Nkx6.1. Thus, Nkx6.1-mediated repression both promotes motor neuron and V2 neural fates and concomitantly prevents ventral progenitors from initiating an aberrant program of V1 neurogenesis. By extension, Gro/TLE-dependent class I proteins, such as Dbx1 and Dbx2, are likely to function in a similar manner to exclude the expression of Nkx6.1 and other repressors of V0 and V1 neuronal fate (Briscoe et al., 2000; Pierani et al., 2001).

Gro/TLE-dependent class I proteins, such as Dbx1 and Dbx2, are likely to function in a similar manner as class II proteins to exclude the expression of Nkx6.1 and other repressors of V0 and V1 neuronal fate (Briscoe et al., 2000; Pierani et al., 2001). However, many transcription factors fulfils both activator and repressor functions, and we cannot exclude that class I proteins have also an activator function in neuronal cell fate specification. In this case, the role of class I proteins in neuronal patterning would resemble the role of the homeodomain protein Pax5 in B-lymphoid cell differentiation. The expression of Pax5 in B-lymphoid progenitors function to restricting the developmental potential of these cells by suppressing alternative cell fates, an activity that might be mediated through the ability of Pax5 to recruit Gro/TLE proteins (Eberhard et al., 2000). However, Pax5 also activates expression of certain B-cell lineage genes, and thus its role in B-cell lineage specification appears to require both its activator and repressor functions.

Our results indicate that Nkx2.2-mediated repression also serves a dual role in the control of neuronal cell fate, analogous to that described for Nkx6.1. The repressor activity of Nkx2.2 suppresses motor neuron differentiation and permits cells to adopt V3 identity. However, within the p3 domain Nkx6.1 and Nkx2.2 are coexpressed by individual progenitor cells (Briscoe et al., 1999), yet the activity of Nkx2.2 is dominant over Nkx6.1. How then does the activity of Nkx2.2 override that of Nkx6.1 to ensure that progenitors in this domain do not initiate motor neuron fate? One possibility is that Nkx2.2 has higher affinity than Nkx6.1 for Gro/TLE proteins, and sequesters Gro/TLE corepressors within p3 progenitors, thus eliminating Nkx6.1 function. Against this idea, Dbx2 is ectopically expressed in p3 domain progenitors in Nkx6.1 mutants, indicating that Nkx6.1 is functional also in p3 progenitors (Sander et al., 2000). Thus, a more likely explanation is that Nkx2.2 block motor neuron generation in the p3 domain by repressing directly the expression genes, such as MNR2 and Lim3, that acts downstream of Nkx6.1 to establish motor neuron identity in the adjacent pMN domain (Briscoe et al., 1999; 2000).

The finding that class II proteins, and most likely also class I proteins, specify neuronal identity through repression, raises the issue of the steps that activate determinants, such as MNR2 and Lim3, within individual progenitor domains. At one extreme, the activation of distinct subtype determinant genes in cells in individual progenitor domains could be achieved by a single common activator protein uniformly expressed along the entire dorsoventral axis of the neural tube. In this view, the ability of this activator to induce distinct subtype determinant genes would be constrained by the repertoire of cis-acting binding sites for class I and class II proteins found in the regulatory regions of individual neural subtype determinant genes. Thus, the specificity of neuronal subtype generation would emerge solely through the activity of repressors. In support for such idea, the Gro/TLE proteins are considered to mediate “active” repression that silences gene expression largely independent of promoter context. An alternative extreme view is that each individual progenitor domain expresses a distinct activator protein. In this case, the pattern of activator expression is likely to be defined as a strict response to the repressor activities of class I and class II homeodomain proteins.

Determining between these two extreme views is not possible at present since the identity of proteins that activate the expression of neuronal subtype determinants is known. Several members of the basic helix-loop-helix (bHLH) class of transcriptional activators are expressed in discrete domains along the dorsoventral axis of the caudal neural tube (Sommer et al., 1996, Ma et al., 1997 ). Some of these genes transgress individual progenitor domain boundaries (Sommer et al., 1996, Ma et al., 1997) whereas other are more restricted to specific individual ventral progenitor domains (Sommer et al., 1996; Briscoe et al., 1999). Determining whether bHLH proteins mediate the missing activator function, and how they may integrate with class I and class II mediated repression in the spatial control of neuronal fate specification seems important for a further understanding of ventral neuronal patterning.

Taken together, this analysis of class II and class I protein function places derepression at the core of ventral cell fate specification. Our results raise the possibility that progenitor cells in the developing CNS that posses the potential to generate a wide array of distinct neuronal subtypes, are restricted to specific fates through repressors that suppress all but one program of neuronal differentiation. In this context, we note that several transcription factors with restricted expression along the rostrocaudal axis, including members of the En-, Gbx- and Pax-families also possesses Gro/TLE binding motifs. Thus, rostrcaudal restrictions of motor neuron and ventral interneuron generation may involve Gro/TLE-mediated repression through mechanisms similar to those operating along the dorsoventral axis of the neural tube. Also, the detection of eh1 domains in the vast majority of identified Nkx proteins, and in other homeodomain proteins with functions in specifying of non-neuronal cell types, suggests that the derepression mechanism at work in the ventral neural tube may have a more pervasive role in cell fate control in embryonic development.

Cell Fate—Guided Differentiation into Specific Cell Types

A primary role of GIPs appears to be to exclude other repressor proteins from progenitor cells, and in such way permitting the activation of specific downstream determinants that establishes the subtype identity of neurons. In principle, any given progenitor cell in the developing CNS might posses the potential to generate a wide array of distinct neuronal subtypes, with the pluripotentiality of cells restricted by repressors that suppress all but one differentiation program. GIPS also are involved in guiding the determination of a variety of cell types other than neuronal cells.

Cell fate can be influenced by a variety of factors. As used herein, “differentiation” refers to the expression or manifestation of the fate of a particular cell. Any protein or polypeptide that specifically induces or influences differentiation is commonly referred to as a differentiation factor.

Groucho interacting proteins (“GIPs”) are proteins that interact with Groucho. Groucho is a neurogenic gene and member of the Enhancer of split complex (E[sp1]-C). Groucho is named for a mutant that developed an increased number of supraorbital bristles (bristles around the eye), reminiscent of the bushy browed Marx brother (Schrons, H. Knust, E., and Campos-Ortega, J. A., Genetics: 132: 481-503, 1992). It encodes a nuclear protein expressed ubiquitously in both embryos and imaginal discs, and acts as a transcriptional repressor of several important genes in Drosophila development.

Groucho interacts with helix-loop-helix protein Hairy, one of the Enhancer of split complex genes, and Deadpan, and thus regulates transcription as a transcriptional corepressor, in partnership with other proteins. Groucho-E(SPL) protein complexes promote epidermal cell fate by repressing transcription of proneural AS-C genes (Paroush, Z., et al., Cell: 79: 805-815, 1994). In wing discs, hedgehog and entrailed are repressed in anterior cells by the activity of Groucho (de Celis, J. F. and Rulz-Gomez, M., Development: 121:

3467-76,1995). Thus Groucho, lacking a DNA binding domain, acts as a transcription factor by combining with other transcription factors to form an active complex repressing the transcription of target genes.

A transgenic embryo assay has been employed to discover the mode of repression mediated by Hairy. Hairy can act as a dominant repressor capable of functioning over long distances to block multiple enhancers. Hairy is shown to repress a heterologous enhancer, the rhomboid enhancer sequence, when bound 1 kb from the nearest upstream activator.

Hairy has been shown to interact with the co-repressor protein Groucho through the C-terminal WRPW motif. Gro is not known to bind DNA, but fusions of GRO with heterologous DNA binding domains have revealed that GRO can act as a transcriptional repressor. The Gro protein contains several repeats of a 40-residue motif, termed the WD40 repeat, that is thought to mediate protein-protein interactions. Tup1, a yeast corepressor protein that also contains WD40 repeats, is recruited to DNA by the alpha2 repressor in alpha-type cells for the silencing of alpha-specific genes. Similarly, Hairy may recruit Gro for silencing specific genes in the Drosophila embryo. The yeast mating-type repressors alpha2 and Tup1 have been reported to interact with histones. This observation raised the possibility that Gro mediates transcriptional silencing by influencing chromatin structure.

Hairy-related proteins are site-specific DNA-binding proteins defined by the presence of both a repressor-specific bHLH DNA binding domain and a carboxyl-terminal WRPW (Trp-Arg-Pro-Trp) motif. These proteins act as repressors by binding to DNA sites in target gene promoters and not by interfering with activator proteins, indicating that these proteins are active repressors that should therefore have specific repression domains. The WRPW motif is a functional transcriptional repression domain sufficient to confer active repression to Hairy-related proteins or a heterologous DNA-binding protein, Gal4. The WRPW motif is sufficient to recruit Groucho or the TLE mammalian homologs to target gene promoters. Groucho and TLE proteins actively repress transcription when directly bound to a target gene promoter. Thus Groucho family proteins are active transcriptional corepressors for Hairy-related proteins and are recruited by the 4-amino acid protein-protein interaction domain, WRPW (Fisher, A. L., Ohsako, S. and Caudy, M., Mol. Cell. Biol.:16: 2670-2677, 1996).

Stifani et al. (Nature Genet. 2: 119-127, 1992) described human homologs of Drosophila groucho protein; these were designated TLE for ‘transducin-like enhancer of split.’ Miyasaka et al. (Europ. J. Biochem. 216: 343-352, 1993) reported the cDNA cloning, nucleotide and deduced amino acid sequencing, and tissue-specific expression of mouse and human TLE genes (also known as ESG for ‘enhancer of split groucho’). By Southern blot analysis of genomic DNA from human/Chinese hamster somatic hybrid cell lines, Miyasaka et al. mapped the human TLE1 gene to chromosome 9 and the TLE3 gene to chromosome 15. Although they mapped the TLE3 gene to 15q22, Liu et al. (Genomics 31: 58-64, 1996) found that the TLE1 and TLE2 genes are organized in a tandem array on 19p13.3. These assignments were determined by fluorescence in situ hybridization (FISH). See, generally, OMIM 600189.

Liu et al. showed that expression of individual TLE genes correlated with immature epithelial cells that are progressing toward that terminally differentiated state, suggesting a role during epithelial differentiation. In both normal tissues and tissues resulting from incorrect or incomplete maturation events (such as metaplastic and neoplastic transformations), TLE expression was elevated and coincided with ‘Notch’ expression, implicating these molecules in the maintenance of the undifferentiated state in epithelial cells. By FISH, Liu et al. found a TLE-related gene on chromosome 9q22 and concluded that it represents a new TLE gene or a pseudogene.

Groucho interacting proteins (“GIPs”) are proteins that interact with Groucho. The identity of a particular GIP influences the fate of a given cell. In other words, the identity of the GIP determines what the final cell type will be. Thus, GIP functions as a differentiation factor.

Transcription factors are examples of GIPs. Those skilled in the art will recognize that transcription factors include regulatory proteins that bind to regulatory regions of eukaryotic genes and interact with each other and with RNA polymerase to modulate transcription. In addition, transcription factors also interact with Groucho. Transcription factors include, but are not limited to, homeodomain proteins.

Homeodomain proteins are proteins containing the homeodomain, a 60 amino acid protein motif encoded by the homeobox, which is a semi-conserved 180 nucleotide DNA sequence found within the coding region of many eukaryotic genes. As noted above, homeodomain proteins function as transcription factors and, thus, are GIPs.

The influence exerted by GIPs on cell fate and differentiation can vary by both cell type and by GIP identity. For example, in case of motor neurons, the primary role of the GIP Nkx6.1 in the generation of somatic motor neurons appears to be to permit the activation of motor neuron determinants, such as MNR2 and Lim3, during the final cell division of motor neuron progenitors. The activation of motor neuron determinants in most neural progenitor cells is normally repressed by transcription factors that recruit Gro/TLE corepressors. Our data suggest that different genes subserve this repressor function in different classes of neural progenitor cells. Nkx6.1 repressed factors, including Dbx2 and perhaps Gsh1/2, appear to suppress the initiation of motor neuron fate in the intermediate and dorsal regions of the neural tube, whereas Nkx2.2 repress motor neuron generation ventrally in p3 progenitors.

As another example, transcription factors such as the homeodomain proteins, have been shown to play an important role in pancreatic development. See Sussel et al., Development 125(12):2213-21 (1988), incorporated herein by reference. The pancreas is organized into cluster of cells known as islets of Langerhans, which include four well-defined cell types called alpha, beta, delta, and PP. A member of the mammalian NK2 homeobox transcription factor family, Nkx2.2, is expressed in all of these cell types except the delta cells. See id. Mice homozygous for a null mutation of Nkx2.2 develop severe hyperglycemia and die as a result of a lack of beta cells. See id. Thus, it has been proposed that Nkx2.2 is required for the final differentiation of pancreatic beta cells. See id.

Moreover, it has also been shown that disruption of the homeobox gene Nkx6.1 in mice leads to a loss of beta cell precursors and blocks beta-cell neogenesis. See Sander et al., Development 127(24):5533-40 (2000), incorporated herein by reference. In contrast, islet development in Nkx6.1/Nkx2.2 double mutant embryos is identical to Nkx2.2 single mutant islet development. See id. Thus, there are apparently two independently controlled beta-cell differentiation pathways. See id. The GIP Nkx6.1 appears to function downstream from the GIP Nkx2.2 in the beta-cell differentiation pathway.

Another example of the influence of GIP identity on final differentiated cell type occurs in the differentiation of glial cell precursors into oligodendrocytes. It has been demonstrated in the developing chicken central nervous system (CNS) as well as in the rodent CNS that the homeodomain transcription factor Nkx2.2 regulates that differentiation and/or maturation of oligodendrocytes. See Qi et al., Development 128(14):2723-33 (2001). Thus, in the CNS, Nkx2.2 appears to have a role in the regulation of oligogliogenesis. See id.

Other non-limiting examples of the influence of GIPs on cell fate and differentiation are provided in the following Table A:

| | Cell/Tissue | | | |
|---|---|---|---|---|
| GIP | Type | Organism | Effect | Reference |
| Nkx2.5 | myocardial | embryonic | role in the regulation | Thomas et al., |
| | conduction | chick; | and/or maintenance of | Anat Rec |
| | cells | fetal | specialized fate | 263(3):307-13 |
| | | mouse; | selection by embryonic | (2001) |
| | | human | myocardial cells | |
| Nkx2.2 | retina | avian | migration of cells | Fu et al., Brain |
| | | | from optic nerve to | Res Dev Brain |
| | | | retina | Res 129(1):115- |
| | | | | 18 (2001) |
| Pax4 | pancreatic | mouse | essential for | Xu et al., Mol |
| | islet cells | | differentiation of islet | Cell Endocrinol |
| | | | cells; mutant mice lack | 170(1-2):79-89 |
| | | | mature beta and delta | (2000) |
| | | | islet cells | |
| Fetoprotein | liver | mouse | activates the alpha(1) | Pare et al., J. |
| transcription | | | fetoprotein gene during | Biol Chem |
| factor (FTF) | | | early liver | 276(16):13136- |
| | | | developmental growth | 44 (2001) |
| Nkx2-1 | smooth | mouse | expressed in vascular | Carson et al., J. |
| | muscle | | and visceral | Biol Chem |
| | cell | | mesoderm-derived | 275(50):39061- |
| | | | muscle tissues and may | 72 (2000) |
| | | | influence smooth muscle | |
| | | | cell differentiation | |
| Nkx6.1 | motor | | persistent and robust | Cai et al., Genesis |
| | neurons | | expression in motor | 27(1):6-11 |
| | | | neurons and mesenchymal | (2000)36 |
| | | | cells suggests an | |
| | | | important role in | |
| | | | controlling cell | |
| | | | fate specification and | |
| | | | differentiation | |
| Nkx2.5 | heart | chicken | involved in cardiac | Searcy et al., Dev |
| | | | commitment and | Dyn 213(1):82- |
| | | | differentiation | 91 (1998) |
| Nkx3.1 | prostate | | may play a prominent | Bieberich et al., J. |
| | | | role in prostate | Biol. Chem |
| | | | development and in the | 271(50)31779-82 |
| | | | maintenance of the | (1996) |
| | | | differentiated state of | |
| | | | prostatic epithelial cells | |

Compositions Involved in Mediating Guided Cell Fate Determination

In another aspect, the invention is directed to novel compositions involved in the molecular interactions necessary to guide the fate of cellular differentiation. The critical molecular interactions occur between Groucho-interacting proteins (GIP) and Groucho corepressors. More specifically, these Groucho-interacting proteins include polypeptides with a functional domain that mediates neural patterning activity and a novel Groucho-interacting protein named Nkx6.3. Compositions of the present invention also include novel complexes formed between a class of transcriptional repressors and a corepressor which they recruit. This recruitment of a corepressor by the GIPs demonstrates that GIPs help guide cellular differentiation by acting as repressors.

GIP-Groucho Formation Via the TN Domain

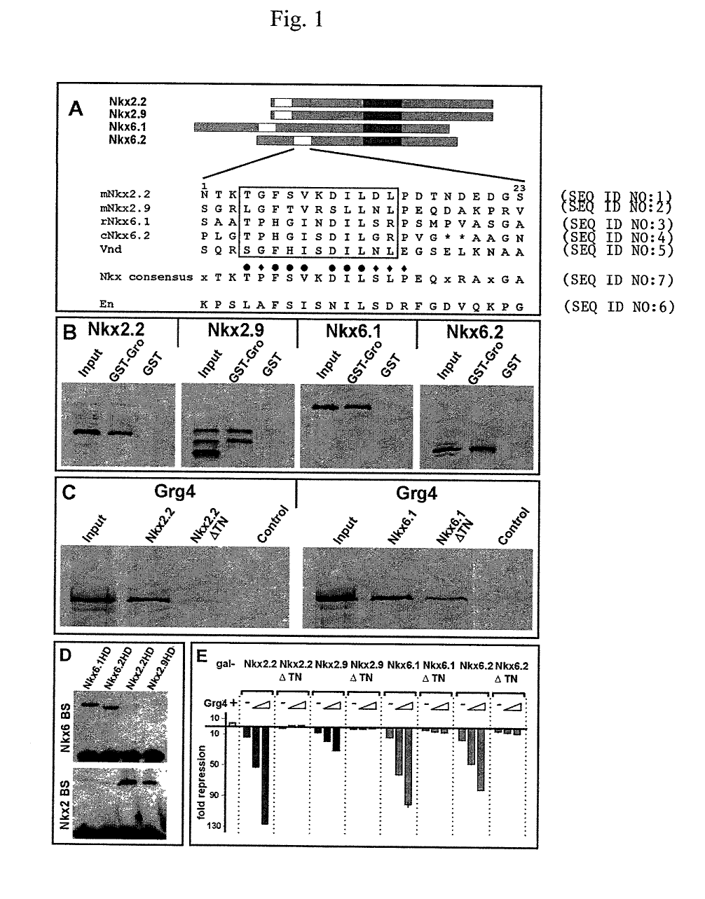

One aspect of this invention provides functional domains that mediate the neural patterning activity of the transcription factors which help guide the fate of cellular differentiation. One such functional domain is the TN (NK decapeptide) domain. The present invention provides a consensus sequence for this domain which is involved with the recruitment of a groucho corepressor. The consensus sequence is provided herein as SEQ ID NO: 7. See also FIG. 1 and Example II.

Nkx6.3 as Novel GIP

Another composition of the present invention is a novel Groucho-interacting protein which helps guide the fate of cellular differentiation by recruiting a Groucho-corepressor. The novel transcription factor is termed Nkx6.3 and is provided herein as SEQ ID NO: 12 (nucleic acid) and 13 (amino acid). See also Example IX.

GIP-Groucho Complex

Yet another aspect of the present invention is the complex formed between the groucho-interacting protein and the groucho-corepressor proteins, with such complex playing a critical role in guiding the fate of cells in cellular differentiation.

Details regarding the above-described compositions involved in guiding the fate of cellular differentiation are provided below.

The present invention is based in part on the identification of a novel interaction between Groucho and a Groucho Interacting Protein, or GIP. For example, the GIP can include a TN domain such as an Nkx consensus sequence as shown in SEQ ID NO: 7 or an En domain as shown in SEQ ID NO: 6. In some embodiments, the GIP is a known protein, for example the GIP can be a transcription factor, such as a homoedomain protein. In other embodiments, the GIP can be a newly identified protein, such as Nkx6.3. The nucleic acid sequence of a cDNA encoding a Nkx6.3 polypeptide is shown in Table 1 as SEQ ID NO: 12. The amino acid sequence of the polypeptide encoded by this nucleic acid sequence is shown as SEQ ID NO: 13.

Included within the invention are Groucho-interacting GIP-derived polypeptides, as well as complexes that include Groucho-binding GIP polypeptides and Groucho. Also disclosed are antibodies to these polypeptides and complexes, as well as pharmaceutical compositions and methods utilizing these nucleic acids, polypeptides, and complexes.

GIP Nucleic Acids

Included in the invention is a GIP nucleic acid. By “GIP nucleic acid” is meant a nucleic acid which encodes for a polypeptide that interacts with Groucho. In some embodiments, the GIP nucleic acid encodes a GIP polypeptide. By “GIP polypeptide” is meant a polypeptide at least 70% identical to the amino acid sequence of a polypeptide that includes the amino acid sequence of a protein capable of interacting with Groucho.

In some embodiments, the GIP nucleic acid encodes a transcription factor. The GIP nucleic acid is at least 70% identical to a nucleic acid including the nucleic acid sequence of a known transcription factor. Also included in the term “GIP nucleic acid” is a nucleic acid fragment that includes a portion of a sequence at least 70% identical to a nucleic acid including the nucleic acid sequence of a known transcription factor, provided that the fragment contains enough sequence to specifically hybridize to a sequence at least 70% identical to a nucleic acid including the nucleic acid sequence of a known transcription factor.

In some embodiments, the GIP nucleic acid encodes a GIP polypeptide that includes a Groucho binding domain. By “Groucho-binding domain” is meant a region of amino acids sufficient to allow the polypeptide in which the region of amino acids is present to bind specifically to a Groucho polypeptide. The encoded Groucho-binding polypeptide can be derived from a full-length GIP polypeptide, or from a derivative, fragment, analog, homolog or paralog of a GIP polypeptide. Preferably, the derivative, fragment, analog, homolog or paralog the has one or more of the following attributes: (i) is functionally active (i.e., capable of exhibiting one or more functional activities associated with full-length, wild-type GIP; (ii) possesses the ability to bind the Groucho protein; (iii) is immunogenic or (iv) is antigenic.

In some embodiments, the fragment of a GIP polypeptide includes at least 10, 20, 30, 40, or 50 amino acid residues (preferably not larger that 35, 100 or 200 amino acid residues) of the GIP polypeptide. Derivatives or analogs of the encoded GIP polypeptide include, e.g., molecules which include regions which are substantially homologous to the Groucho protein or GIP in various embodiments, of at least 50%, 60%, 70%, 80%, 90% or 95% amino acid identity when: (i) compared to an amino acid sequence of identical size; (ii) compared to an aligned sequence in which the alignment is done by a computer homology program known within the art or (iii) the encoding nucleic acid is capable of hybridizing to a sequence encoding the Groucho protein or GIP under stringent, moderately stringent, or non-stringent conditions, as is discussed below. The Groucho-binding domain can include the Nkx consensus sequence of SEQ ID NO: 7.

Thus, in some embodiments, the encoded Groucho-binding domain is derived from a GIP polypeptide that includes a sequence that is at least 90% identical to a polypeptide which includes the amino acid sequence of a transcription factor, such as, for example, an Nkx polypeptide. In some embodiments, the domain is derived from a GIP polypeptide which is at least 95, 98 or even 99% identical to a polypeptide including the amino acid sequence of SEQ ID NO: 13.

In some embodiments, the encoded GIP polypeptide is a polypeptide which includes the amino acid sequence of SEQ ID NO: 13. For example, the GIP polypeptide may correspond to some or all of the amino acid sequences encoded by the longest open reading frame present in a nucleic acid that includes SEQ ID NO: 12. Alternatively, the GIP polypeptide can include a region of the amino acid sequence of SEQ ID NO: 13 that is able to bind specifically to a Groucho polypeptide.

Procedures for identifying regions within a protein that bind to Groucho can be readily identified by one of ordinary skill in the art. In addition, the sequence information disclosed herein for a GIP, may be combined with any method available within the art to obtain longer clones encompassing additional GIP coding sequences. Such sequences, for example, may encode an initiator codon for a GIP polypeptide.

For example, the polymerase chain reaction (PCR) may be utilized to amplify the sequence within a cDNA library. Similarly, oligonucleotide primers may also be used to amplify by PCR sequences from a nucleic acid sample (RNA or DNA), preferably a cDNA library, from an appropriate source.

PCR may be performed by use of, for example, a thermal cycler and Taq polymerase. The DNA being amplified is preferably cDNA derived from any eukaryotic species. Several different degenerate primers may be synthesized for use in the PCR reactions. It is also possible to vary the stringency of the hybridization conditions used in priming the PCR reactions, to amplify nucleic acid homologs by allowing for greater or lesser degrees of nucleotide sequence similarity between the known nucleotide sequence and the nucleic acid homolog being isolated. For cross species hybridization, low stringency conditions are preferred; whereas for same species hybridization, moderately stringent conditions are preferred.

Any eukaryotic cell may potentially serve as the nucleic acid source for the molecular cloning of the GIP sequences. The DNA may be obtained by standard procedures known in the art from cloned DNA (e.g., a DNA “library”), by chemical synthesis, by cDNA cloning, or by the cloning of genomic DNA, or fragments thereof, purified from the desired cell. See e.g., Sambrook, et al., 1989. Molecular Cloning: A Laboratory Manual, 2nd ed, (Cold Spring Harbor Laboratory Press, Cold Spring Harbor, N.Y.); and Glover, 1985. DNA Cloning: A Practical Approach (MRL Press, Ltd., Oxford, U.K. Vol. I, II). Clones derived from genomic DNA may contain regulatory and intronic DNA regions in addition to exonic (coding) regions; whereas clones derived from cDNA will contain only exonic sequences.

GIP nucleic acids are preferably derived from a cDNA source. Identification of the specific cDNA containing the desired sequence may be accomplished in a number of ways. In one method, a portion of the GIP sequence (e.g., a PCR amplification product obtained as described above), or an oligonucleotide possessing a sequence of a portion of the known nucleotide sequence, or its specific RNA, or a fragment thereof, may be purified, amplified, and labeled, and the generated nucleic acid fragments may be screened by nucleic acid hybridization utilizing a labeled probe. See e.g., Benton & Davis, 1977. Science 196:180. In a second method, the appropriate fragment is identified by restriction enzyme digestion(s) and comparison of fragment sizes with those expected from comparison to a known restriction map (if such is available) or by DNA sequence analysis and comparison to the known nucleotide sequence of GIP. In a third method, the gene of interest may be detected utilizing assays based on the physical, chemical or immunological properties of its expressed product. For example, cDNA clones, or DNA clones which hybrid-select the proper mRNAs, may be selected as a function of their production of a protein which, for example, has similar or identical electrophoretic migration, isoelectric focusing behavior, proteolytic digestion maps, antigenic properties or ability to bind the Groucho protein. In a fourth method, should an anti-GIP antibody be available, the protein of interest may be identified by the binding of a labeled antibody to the putatively GIP clone in an enzyme-linked immunosorbent assay (ELISA).

The GIP nucleic acid can be an isolated nucleic acid. An “isolated” nucleic acid molecule is one that is separated from other nucleic acid molecules which are present in the natural source of the nucleic acid. Examples of isolated nucleic acid molecules include, e.g., recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated GIP nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule including the nucleotide sequence of a GIP, and/or encoding the polypeptide including the amino acid sequence of a GIP, or a complement of any nucleotide sequence, can be isolated using standard molecular biology techniques and the sequence information provided herein. Using all or a portion of the nucleic acid sequences of a GIP as a hybridization probe, GIP nucleic acid sequences can be isolated using standard hybridization and cloning techniques (e.g., as described in Sambrook et al., eds., MOLECULAR CLONING: A LABORATORY MANUAL 2nd Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.).

In some embodiments, the GIP nucleic acid is an Nkx nucleic acid identical in sequence to a nucleic acid that includes SEQ ID NO: 7. In other embodiments, the GIP nucleic acid is an Nkx nucleic acid, such as a nucleic acid that includes SEQ ID NO: 12. In other embodiments, the GIP nucleic acid differs from the nucleic acid sequence of a nucleic acid that includes SEQ ID NO: 12. For example, the GIP nucleic acid may include a sequence at least 90%, 95%, 98%, or even 99% or more identical to the nucleic acid sequence of SEQ D NO: 12. These sequences are referred to herein as variant Nkx6.3 nucleic acid sequences. An alternative way to describe variant GIP nucleic acid sequences is to describe nucleic acids that hybridize to a sequence including SEQ ID NO: 12, or to a Groucho-binding fragment of SEQ ID NO: 12.

To determine the percent relatedness of two nucleic acid sequences, or of two amino acid sequences (e.g., as would be done to compare variant GIP polypeptides as discussed in more detail below, or nucleic acids encoding such variant polypeptides), the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

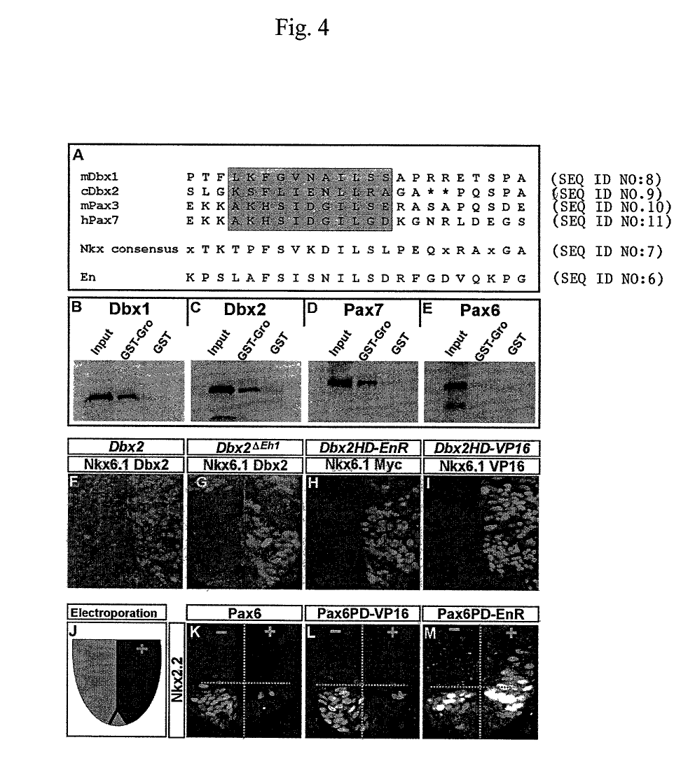

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence including the nucleic acid of a GIP or a polypeptide including the amino acid sequence shown in FIG. 1A or FIG. 4A.

The term “sequence identity” refers to the degree to which two polynucleotide or polypeptide sequences are identical on a residue-by-residue basis over a particular region of comparison. Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (ie., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

DNA sequence polymorphisms that lead to changes in the amino acid sequences of a GIP polypeptide may exist within a population (e.g., the human population). Such genetic polymorphism in the GIP gene may exist among individuals within a population due to natural allelic variation. As used herein, the terms “gene” and “recombinant gene” refer to nucleic acid molecules comprising an open reading frame encoding a GIP polypeptide, preferably a mammalia GIP polypeptide. Such natural allelic variations can typically result in 1-5% variance in the nucleotide sequence of the polypeptide gene. Any and all such nucleotide variations and resulting amino acid polymorphisms in GIP that are the result of natural allelic variation and that do not alter the functional activity of GIP are intended to be within the scope of the invention.

Moreover, nucleic acid molecules encoding GIP proteins from other species, are intended to be within the scope of the invention. Nucleic acid molecules corresponding to natural allelic variants and homologues of the GIP cDNAs can be isolated based on their homology to the GIP nucleic acids disclosed herein using the human cDNAs, or a portion thereof, as a hybridization probe according to standard hybridization techniques under stringent hybridization conditions. For example, a soluble huma GIP cDNA can be isolated based on its homology to human membrane-bound GIP. Likewise, a membrane-bound huma GIP cDNA can be isolated based on its homology to soluble huma GIP.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 12. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding GIP proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2× SSC, 0.01% BSA at 50° C. An isolated nucleic acid molecule of the invention that hybridizes under stringent conditions to the sequence of SEQ ID NO: 12 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 12, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of SEQ ID NO: 12, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

In addition to naturally-occurring allelic variants of the GIP sequence that may exist in the population, a GIP nucleic acid also includes nucleic acids including changes introduced by alteration of the nucleotide sequence of SEQ ID NO: 12. In some embodiments, these changes lead to changes in the amino acid sequence of the encoded GIP protein, without altering the functional ability of the GIP protein. For example, nucleotide substitutions leading to amino acid substitutions at “non-essential” amino acid residues can be made in the sequence of SEQ ID NO: 13. A “non-essential” amino acid residue is a residue that can be altered from the wild-type sequence of GIP without altering the biological activity, whereas an “essential” amino acid residue is required for binding to Groucho or for a biological activity mediated by a GIP polypeptide. For example, amino acid residues that are conserved among the GIP proteins of the present invention, are predicted to be particularly unamenable to alteration.

In addition, amino acid residues that are conserved among family members of the GIP proteins of the present invention, are also predicted to be particularly unamenable to alteration. Other amino acid residues, however, (e.g., those that are not conserved or only semi-conserved among members of the GIP proteins) may not be essential for activity and thus are likely to be amenable to alteration.

Another aspect of the invention pertains to nucleic acid molecules encoding GIP proteins that contain changes in amino acid residues that are not essential for activity. Such GIP proteins differ in amino acid sequence from wild type GIP, yet retain biological activity (e.g., Groucho-binding activity). In one embodiment, the isolated nucleic acid molecule comprises a nucleotide sequence encoding a protein, wherein the protein comprises an amino acid sequence at least about 45% homologous to the amino acid sequence of a known GIP. Preferably, the protein encoded by the nucleic acid molecule is at least about 60% homologous to a known GIP, more preferably at least about 70%, 80%, 90%, 95%, 98%, and most preferably at least about 99% homologous to a known GIP.

For example, an isolated nucleic acid molecule encoding a GIP protein homologous to the protein of SEQ ID NO: 13 can be created by introducing one or more nucleotide substitutions, additions or deletions into the nucleotide sequence of SEQ ID NO: 13, such that one or more amino acid substitutions, additions or deletions are introduced into the encoded protein.

Mutations can be introduced into SEQ ID NO: 12 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in GIP is replaced with another amino acid residue from the same side chain family. Alternatively, in another embodiment, mutations can be introduced randomly along all or part of a GIP coding sequence, such as by saturation mutagenesis, and the resultant mutants can be screened for GIP biological activity to identify mutants that retain activity. Following mutagenesis of SEQ ID NO: 12, the encoded protein can be expressed by any recombinant technology known in the art and the activity of the protein can be determined.

In one embodiment, a mutant GIP polypeptide can be assayed for (1) the ability to form protein:protein interactions with other GIP proteins, or biologically active portions thereof; (2) the ability to form complexes with Groucho polypeptides, (3) the ability to form complexes with a mutant GIP protein and a GIP ligand (including GIP binding domains of Groucho polypeptides, or other GIP ligands); (4) the ability of a mutant GIP protein to bind to an intracellular target or biologically active portion thereof, (e.g., DNA binding proteins); (5) the ability to bind DNA; or (6) the ability to specifically a GIP protein antibody.

If desired, GIP nucleic acids can be derived from a cDNA source. Identification of the specific cDNA containing the desired sequence may be accomplished in a number of ways. In one method, a portion of the GIP sequence (e.g., a PCR amplification product), or an oligonucleotide possessing a sequence of a portion of the known nucleotide sequence, or its specific RNA, or a fragment thereof, may be purified, amplified, and labeled, and the generated nucleic acid fragments may be screened by nucleic acid hybridization utilizing a labeled probe. See e.g., Benton & Davis, Science 196:180, 1977. In a second method, the appropriate fragment is identified by restriction enzyme digestion(s) and comparison of fragment sizes with those expected from comparison to a known restriction map (if such is available) or by DNA sequence analysis and comparison to the known nucleotide sequence of GIP. In a third method, the gene of interest may be detected utilizing assays based on the physical, chemical or immunological properties of its expressed product. For example, cDNA clones, or DNA clones which hybrid-select the proper mRNAs, may be selected as a function of their production of a protein which, for example, has similar or identical electrophoretic migration, isolectric focusing behavior, proteolytic digestion maps, antigenic properties or ability to bind the Groucho protein. In a fourth method, should an anti-GIP antibody be available, the protein of interest may be identified by the binding of a labeled antibody to the putatively GIP clone in an enzyme-linked immunosorbent assay (ELISA).

Also included in the invention are vectors that include GIP nucleic acids, as well as cells containing GIP nucleic acids or vectors containing GIP nucleic acids. In general, any suitable vector known in the art can be used. For recombinant expression of a GIP polypeptide, a nucleic acid containing all or a portion of the nucleotide sequence encoding the polypeptide may be inserted into an appropriate expression vector (i.e., a vector which contains the necessary elements for the transcription and translation of the inserted protein coding sequence). The regulatory elements can be heterologous (i.e., not the native gene promoter), or can be supplied by the native promoter for the GIP polypeptide, or any GIP genes and/or their flanking regions.

Exemplary host-vector systems that may be used to propagate GIP nucleic acids, or to express GIP polypeptides include, e.g.: (i) mammalian cell systems which are infected with vaccinia virus, adenovirus; (ii) insect cell systems infected with baculovirus; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

The expression of the specific proteins may be controlled by any promoter/enhancer known in the art including, e.g.: (i) the SV40 early promoter (see e.g., Bernoist & Chambon, Nature 290:304-310, 1981); (ii) the promoter contained within the 3′-terminus long terminal repeat of Rous Sarcoma Virus (see e.g., Yamamoto, et al., Cell 22:787-797, 1980); (iii) the Herpesvirus thymidine kinase promoter (see e.g., Wagner, et al., Proc. Natl. Acad. Sci. USA 78:1441-1445, 1981); (iv) the regulatory sequences of the metallothionein gene (see e.g., Brinster, et al., Nature 296:39-42, 1982); (v) prokaryotic expression vectors such as the β-lactamase promoter (see e.g., Villa-Kamaroff, et al., Proc. Natl. Acad. Sci. USA 75:3727-3731, 1978); (vi) the tac promoter (see e.g., DeBoer, et al., Proc. Natl. Acad. Sci. USA 80:21-25, 1983).

Plant promoter/enhancer sequences within plant expression vectors may also be utilized including, e.g.,: (i) the nopaline synthetase promoter (see e.g., Herrar-Estrella, et al., Nature 303:209-213, 1984); (ii) the cauliflower mosaic virus 35S RNA promoter (see e.g., Garder, et al., Nuc. Acids Res. 9:2871, 1981) and (iii) the promoter of the photosynthetic enzyme ribulose bisphosphate carboxylase (see e.g., Herrera-Estrella, et al., Nature 310:115-120, 1984).

Promoter/enhancer elements from yeast and other fungi (e.g., the Gal4 promoter, the alcohol dehydrogenase promoter, the phosphoglycerol kinase promoter, the alkaline phosphatase promoter), as well as the following animal transcriptional control regions, which possess tissue specificity and have been used in transgenic animals, may be utilized in the production of proteins of the present invention.

Other animal transcriptional control sequences derived from animals include, e.g.,: (i) the insulin gene control region active within pancreatic β-cells (see e.g., Hanahan, et al., Nature 315:115-122, 1985); (ii) the immunoglobulin gene control region active within lymphoid cells (see e.g., Grosschedl, et al., Cell 38:647-658, 1984); (iii) the albumin gene control region active within liver (see e.g., Pinckert, et al., Genes and Devel. 1:268-276, 1987); (iv) the myelin basic protein gene control region active within brain oligodendrocyte cells (see e.g., Readhead, et al., Cell 48:703-712, 1987); and (v) the gonadotrophin-releasing hormone gene control region active within the hypothalamus (see e.g., Mason, et al., Science 234:1372-1378, 1986).

In one embodiment, the vector includes a promoter operably-linked to nucleic acid sequences which encode a GIP polypeptide, or a fragment, derivative or homolog, thereof, one or more origins of replication, and optionally, one or more selectable markers (e.g., an antibiotic resistance gene). If desired, a vector is utilized which includes a promoter operably-linked to nucleic acid sequences encoding both the Groucho protein and GIP, one or more origins of replication, and, optionally, one or more selectable markers.

In a specific embodiment, a nucleic acid encoding a GIP polypeptide, or a portion thereof, is inserted into an expression vector. The expression vector is generated by subcloning the sequences into the EcoRI restriction site of each of the three available pGEX vectors (glutathione S-transferase expression vectors; see e.g., Smith et al., Gene 7:31-40, 1988), thus allowing the expression of products in the correct reading frame. Expression vectors that contain the sequences of interest may be identified by three general approaches: (i) nucleic acid hybridization, (ii) presence or absence of “marker” gene function and/or (iii) expression of the inserted sequences. In the first approach, GIP may be detected by nucleic acid hybridization using probes comprising sequences homologous and complementary to the inserted sequences of interest. In the second approach, the recombinant vector/host system may be identified and selected based upon the presence or absence of certain “marker” functions (e.g., binding to an antibody specific for the GIP polypeptide, resistance to antibiotics, occlusion-body formation in baculovirus, and the like) caused by the insertion of the sequences of interest into the vector.

Expression from certain promoters may be enhanced in the presence of certain inducer agents, thus facilitating control of the expression of the GIP polypeptide.

A host cell strain may be selected which modulates the expression of GIP sequences, or modifies/processes the expressed proteins in a desired manner. Moreover, different host cells possess characteristic and specific mechanisms for the translational and post-translational processing and modification (e.g., glycosylation, phosphorylation, and the like) of expressed proteins. Appropriate cell lines or host systems may thus be chosen to ensure the desired modification and processing of the foreign protein is achieved. For example, protein expression within a bacterial system can be used to produce an unglycosylated core protein; whereas expression within mammalian cells ensures “native” glycosylation of a heterologous protein.

A GIP nucleic acid can be engineered so that it encodes a fusion polypeptide having a portion of the GIP polypeptide, e.g., linked to a second polypeptide that includes a sequence that is derived from a polypeptide other than a GIP polypeptide. The second polypeptide can include, e.g., a marker polypeptide or fusion partner. For example, the polypeptide can be fused to a hexa-histidine tag to facilitate purification of bacterially expressed protein or a hemagglutinin tag to facilitate purification of protein expressed in eukaryotic cells.

Also included in the invention is a method of making a GIP polypeptide, e.g., a rat or huma GIP polypeptide, by providing a cell containing DNA encoding a GIP polypeptide and culturing the cell under conditions permitting expression of the GIP encoding DNA, i.e., production of the recombinant GIP by the cell.

Various regions of the GIP nucleic acid can be used to detect GIP nucleic acids in populations of nucleic acids. For example, probes derived from the nucleic acids encoding the Nkx consensus sequence can be used to identify GIP nucleic acids. The probes can be, e.g., hybridization probes derived from these sequences, or primers which specifically amplify these sequences in amplification assays (such as PCR amplification assays).

GIP Polypeptides

Also provided in the invention is a GIP polypeptide, as well as variants and fragments of a GIP polypeptide. The GIP polypeptide, variant or fragment binds a Groucho-polypeptide. For example, the polypeptide can include the Nkx consensus sequence of SEQ ID NO: 7.

In some embodiments, the GIP polypeptide is purified. A “purified” polypeptide, protein or biologically active portion thereof is substantially free of cellular material or other contaminating proteins from the cell or tissue source from which the GIP protein is derived, or substantially free from chemical precursors or other chemicals when chemically synthesized. The language “substantially free of cellular material” includes preparations of GIP protein in which the protein is separated from cellular components of the cells from which it is isolated or recombinantly produced. In one embodiment, the language “substantially free of cellular material” includes preparations of GIP protein having less than about 30% (by dry weight) of non-GIP protein (also referred to herein as a “contaminating protein”), more preferably less than about 20% of non-GIP protein, still more preferably less than about 10% of non-GIP protein, and most preferably less than about 5% non-GIP protein. When the GIP protein or biologically active portion thereof is recombinantly produced, it is also preferably substantially free of culture medium, i e., culture medium represents less than about 20%, more preferably less than about 10%, and most preferably less than about 5% of the volume of the protein preparation.

In some embodiments, the GIP polypeptide, variant, or fragment binds a Groucho polypeptide. In some embodiments, the GIP polypeptide includes an amino acid sequence at least 80% identical to a polypeptide which includes the amino acid sequence of SEQ ID NO: 13. More preferably, the polypeptide is at least 85, 90, 95, 98, or even 99% or more identical. The percent relatedness of two amino acid sequences can be determined as described above for GIP nucleic acids.

The GIP polypeptides can be made by expressing GIP nucleic acids as described above and recovering the GIP polypeptide. Alternatively, the GIP polypeptide can be chemically synthesized using standard techniques, e.g., by the methods described in Solid Phase Peptide Synthesis, 2nd ed., 1984 The Pierce Chemical Co., Rockford, Ill.

The GIP polypeptide can be used to detect a Groucho polypeptide in a biological sample by biological sample, e.g., a cell or tissue sample from a subject, or a cell population cultured in vitro. The sample is contacted with a GIP polypeptide under conditions sufficient to allow for formation of a Groucho-GIP complex (as explained below), if the Groucho polypeptide is present in the sample and then detecting the complex. Presence of the GIP-Groucho complex indicates the Groucho polypeptide is present in the sample.

The GIP polypeptide can also be used to remove, or purify, a Groucho polypeptide from a biological sample. The method includes contacting the sample with a GIP polypeptide under conditions sufficient to allow for formation of a Groucho-GIP complex; if the Groucho polypeptide is present in the sample, and removing the complex from said sample, thereby removing said Groucho polypeptide from said sample.

Preferably, the GIP polypeptide is labeled to facilitate detection and recovery of GIP-polypeptide complexes.

GIP Binding Groucho Polypeptide Derivatives and Fragments

The invention also provides nucleic acids encoding polypeptides or peptides derived from a Groucho polypeptide or peptide. Preferably, the nucleic acids encode polypeptides or peptides that contain a GIP binding domain. Also included in the invention are the polypeptides and peptides encoded by these nucleic acids.

In general, a GIP-binding Groucho polypeptide according to the invention includes any region of a Groucho polypeptide that is less than the length of a full-length Groucho polypeptide, but which includes a GIP binding domain. Preferably, the GIP-binding polypeptide is greater than 5, 6, 7, 8, 9, or 10 amino acids in length and is less than 25, 50, 75, 100, 150, 200, 250, 300, 350, 400 or 450 amino acids in length.

By “Groucho polypeptide” is meant a polypeptide at least 80% identical to the amino acid sequence of a polypeptide that includes an amino acid sequence of a Groucho polypeptide e.g., the nucleic acid sequence of, for example, Grg1, Grg2, Grg3, or Grg4.

By “GIP-binding domain” or “GIP-binding region” is meant a region of amino acids sufficient to allow the polypeptide in which the region of amino acids is present to bind specifically to a GIP polypeptide.

The GIP binding polypeptide present in the complex will typically include at least 6, 8, 10, 12, or 15 or more amino acids of a Groucho polypeptide. Preferably, the polypeptide corresponds to a region of contiguous amino acids in a Groucho polypeptide. The GIP binding polypeptide may be a full-length Groucho polypeptide, e.g., it may have the amino acid sequence of the Groucho polypeptide encoded by a human nucleic acid.

The invention also includes nucleic acids encoding GIP-binding fragments of a Groucho polypeptide. The nucleic acids can include a nucleic acid sequence that is identical to a portion of a huma Groucho polypeptide. Alternatively, the nucleic acid can be a Groucho variant that is greater than 70, 80, 85, 90, 95, 98, or even 99% identical to the corresponding region of the huma Groucho polypeptide nucleic acid. Alternatively, the nucleic acid encodes a GIP-binding Groucho polypeptide which is greater than 70, 80, 85, 90, 95, 98, or even 99% identical to a portion of the huma Groucho polypeptide.

Alternatively, the nucleic acid encoding a GIP-binding Groucho polypeptide may hybridize under low, medium, or high stringency, using the parameters and conditions described above for GIP nucleic acids and polypeptides.

A GIP-binding Groucho nucleic acid nucleic acid can be engineered so that it encodes a fusion polypeptide having a portion of the GIP polypeptide, e.g., linked to a second polypeptide that includes a sequence that is derived from a polypeptide other than a GIP polypeptide. The second polypeptide can include, e.g., a marker polypeptide or fusion partner. For example, the polypeptide can be fused to a hexa-histidine tag to facilitate purification of bacterially expressed protein or a hemagglutinin tag to facilitate purification of protein expressed in eukaryotic cells.

A nucleic acid encoding a GIP binding Groucho peptide can be provided in vector as described above for vectors and cells containing GIP polypeptides. Groucho-binding GIP polypeptides can be synthesized by expressing nucleic acids encoding the GIP binding polypeptides and recovering the expressed polypeptides, e.g., using vectors and/or cells which include nucleic acid encoding a GIP binding Groucho polypeptide.

Also included in the invention is a method of making a Groucho polypeptide fragment, e.g., a rat or huma GIP-binding Groucho polypeptide fragment polypeptide, by providing a cell containing DNA encoding a GIP-binding Groucho polypeptide fragment and culturing the cell under conditions permitting expression of the Groucho fragment encoding DNA, i.e., production of the recombinant Groucho polypeptide by the cell.

Chimeric Polypeptides Including a Groucho-Binding Region of a GIP Polypeptide and a GIP Region of a Groucho Polypeptide

Also included in the invention is a chimeric polypeptide or peptide which includes a region of a Groucho polypeptide covalently linked, e.g., via a peptide bond, to a region of a GIP polypeptide. In some embodiments, the chimeric polypeptide includes six or more amino acids of a Groucho polypeptide covalently linked to six or more amino acids of a GIP polypeptide.

Preferably, the Groucho polypeptide in the chimeric polypeptide includes a GIP-binding domain. Preferably, the GIP polypeptide in the chimeric polypeptide includes a Groucho-binding polypeptide. In some embodiments, the Groucho and GIP polypeptides of the chimeric polypeptide interact to form a complex. Any GIP polypeptide disclosed herein can be used in the complex. For example, the chimeric polypeptide can include an amino acid sequence at least 90% identical to the amino acid sequence of SEQ ID NO: 13. Similarly, any Groucho polypeptide can be present in the chimeric polypeptide.

Also included in the invention are nucleic acids encoding the chimeric polypeptides or peptides, as well as vectors and cells containing these nucleic acids.

The chimeric polypeptides can be constructed by expressing nucleic acids encoding chimeric polypeptides using vectors and cells as described above for GIP polypeptides and Groucho polypeptides, and then recovering the chimeric polypeptides, or by chemically synthesizing the chimeric polypeptides.

Groucho-GIP Complexes

In another aspect, the invention includes a purified complex that includes the Groucho-binding domain of a GIP polypeptide and a GIP-binding domain of a Groucho polypeptide. By purified complex is meant a complex of polypeptide that includes a Groucho-binding domain of a GIP polypeptide and a polypeptide that includes a GIP binding domain of a Groucho polypeptide.

In general, the complex can include any GIP polypeptide described herein as long as it includes a Groucho binding domain. Similarly, any Groucho polypeptide, or any Groucho-derived polypeptide, can be used as long as it contains a GIP binding region.

Thus, the Groucho-binding polypeptide and the GIP-binding polypeptide present in the complex can have the amino acid sequence of a mammalia GIP polypeptide, (e.g., mouse, rat, pig, cow, dog, monkey, frog), or of insects (e.g., fly), plants or, most preferably, human. In some embodiments, both polypeptides have the amino acid sequences of regions of the corresponding human polypeptides. For example, the complex can include a huma Groucho polypeptide, or a GIP binding fragment of a huma Groucho polypeptide, and a huma GIP polypeptide, or a Groucho-binding fragment of a huma GIP polypeptide.

In various embodiments, other components, e.g., polypeptides, are present in the complex in addition to the Groucho-binding domain of a GIP polypeptide and the GIP binding domain of a Groucho polypeptide. In additional embodiments, GIP binding polypeptide and the Groucho-binding polypeptides are the only polypeptide components present in significant levels in the complex. An example of such a complex is a purified complex of a huma Groucho polypeptide and a huma GIP polypeptide.

In some embodiments, the Groucho-binding polypeptide and the GIP binding polypeptide complex is a functionally active complex. As utilized herein, the term “functionally active Groucho-binding polypeptide and the GIP binding polypeptide complex” refers to species displaying one or more known functional attributes of a full-length Groucho protein complexed with full-length GIP. These attributes include, e.g., the control of cellular and physiological processes, such as: (i) control of cell-cycle progression; (ii) cellular differentiation; (iii) regulation of transcription; and (iv) pathological processes including, e.g., medical disorders.

Either, or both, of the GIP or Groucho-binding polypeptides in the complex may be labeled, i.e., attached to one or more detectable substances. Labeling can be performed using any art recognized method for labeling polypeptides. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The complexes can be made by expressing each polypeptide from each nucleic acid and allowing the complex to form from the expressed polypeptides. Any nucleic acid that expresses Groucho-binding GIP polypeptides or GIP biding Groucho polypeptides (or chimerics of these polypeptides) can be used, as can vectors and cells expressing these polypeptides. If desired, the complexes can then be recovered and isolated.

Once a recombinant cell expressing the Groucho protein and/or GIP, or a fragment or derivative thereof, is identified, the individual gene product or complex may be isolated and analyzed. This is achieved by assays that are based upon the physical and/or functional properties of the protein or complex. The assays can include, e.g., radioactive labeling of one or more of the polypeptide complex components, followed by analysis by gel electrophoresis, immunoassay, cross-linking to marker-labeled products. The Groucho protein-GIP complex may be isolated and purified by standard methods known in the art (either from natural sources or recombinant host cells expressing the proteins/protein complex). These methods can include, e.g., column chromatography (e.g., ion exchange, affinity, gel exclusion, reverse-phase, high pressure, fast protein liquid, etc), differential centrifugation, differential solubility, or similar methods used for the purification of proteins.

The Groucho protein-GIP complex is implicated in the modulation of functional activities of the Groucho protein. Such functional activities include, e.g., (i) control of cell-cycle progression; (ii) cellular differentiation; (iii) regulation of transcription; and (iv) pathological processes.

The Groucho protein-GIP complex may be analyzed by hydrophilicity analysis (see e.g., Hopp & Woods, Proc. Natl. Acad. Sci. USA 78:3824-3828, 1981). This analysis can be used to identify the hydrophobic and hydrophilic regions of the proteins, thus aiding in the design of substrates for experimental manipulation, such as in binding experiments, antibody synthesis. Secondary structural analysis may also be performed to identify regions of the Groucho protein and/or GIP which assume specific structural motifs. See e.g., Chou & Fasman, Biochem. 13:222-223, 1974. Manipulation, translation, secondary structure prediction, hydrophilicity and hydrophobicity profiles, open reading frame prediction and plotting, and determination of sequence homologies, can be accomplished using computer software programs available in the art.

Antibodies to GIP Polypeptides, GIP Binding Polypeptides of Groucho Polypeptides, and Complexes of GIP Polypeptides and Groucho Polypeptides

The invention further encompasses antibodies and antibody fragments (such as Fab or (Fab)₂ fragments) that bind specifically to any of the polypeptides or complexes described herein. By “specifically binds” is meant an antibody that recognizes and binds to a particular antigen, e.g., a GIP polypeptide of the invention, but which does not substantially recognize or bind to other molecules in a sample, e.g., a biological sample, which includes a GIP polypeptide.

These polypeptides and complexes can include, e.g., a GIP polypeptide, a GIP binding Groucho polypeptide, a chimeric Groucho-GIP polypeptide, or a complex of a GIP polypeptide and Groucho polypeptide. The antibodies and antibody fragments can alternatively be raised against variants or fragments of the complexes or polypeptides. For example, a purified GIP polypeptide, or a portion, variant, or fragment thereof, can be used as an immunogen to generate antibodies that bind GIP using standard techniques for polyclonal and monoclonal antibody preparation.

A full-length GIP polypeptide can be used, if desired. Alternatively, the invention provides antigenic peptide fragments of GIP polypeptides for use as immunogens. In some embodiments, an antigenic GIP peptide includes at least four amino acid residues of the amino acid sequence shown in SEQ ID NO: 7. The antigenic peptide encompasses an epitope of GIP such that an antibody raised against the peptide forms a specific immune complex with GIP. In some embodiments, the antigenic peptide includes at least 6, 8, 10, 15, 20, or 30 or more amino acid residues of a GIP polypeptide. In one embodiment, epitopes encompassed by the antigenic peptide are regions of GIP that bind Groucho, or are located on the surface of the protein, e.g., hydrophilic regions.

In some embodiments, the antibodies are raised against a GIP-Groucho complex.

Preferably, the anti-complex antibodies bind with higher affinity to the complex as compared to their affinity for an isolated Groucho polypeptide or an isolated GIP polypeptide.

If desired, peptides containing antigenic regions can be selected using hydropathy plots showing regions of hydrophilicity and hydrophobicity. These plots may be generated by any method well known in the art, including, for example, the Kyte Doolittle or the Hopp Woods methods, either with or without Fourier transformation. See, e.g., Hopp and Woods, Proc. Nat. Acad. Sci. USA 78:3824-3828, 1981; Kyte and Doolittle, J. Mol. Biol. 157:105-142, 1982, each incorporated herein by reference in their entirety.

GIP polypeptides (e.g., those including SEQ ID NO: 7), or derivatives, fragments, analogs or homologs thereof, may be utilized as immunogens in the generation of antibodies that specifically bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as GIP, a GIP binding polypeptide fragment of Groucho, or a complex including the two polypeptides. Such antibodies include, e.g., polyclonal, monoclonal, chimeric, single chain, Fab and F_{(ab′)2} fragments, and an Fab expression library. In specific embodiments, antibodies to huma GIP polypeptides or complexes of huma GIP and Groucho polypeptides are disclosed.

Various procedures known within the art may be used for the production of polyclonal or monoclonal antibodies. For example, for the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed GIP, Groucho, or chimeric polypeptide including the two polypeptides, or a complex including the two polypeptides. Alternatively, the immunogenic polypeptide or polypeptides may be chemically synthesized.

The preparation can further include an adjuvant. Various adjuvants used to increase the immunological response include, e.g., Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against GIP, Groucho, chimeras, or complexes can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope GIP, Groucho, chimeras, or complex of these polypeptides. A monoclonal antibody composition thus typically displays a single binding affinity for a particular protein with which it immunoreacts. For preparation of monoclonal antibodies directed towards a particular GIP polypeptide, Groucho polypeptide, chimeras, or complex, or derivatives, fragments, analogs or homologs thereof, any technique that provides for the production of antibody molecules by continuous cell line culture may be utilized. Such techniques include, e.g., the hybridoma technique (see Kohler & Milstein, Nature 256:495-497, 1975); the trioma technique; the human B-cell hybridoma technique (see Kozbor, et al., Immunol Today 4:72, 1983) and the EBV hybridoma technique to produce human monoclonal antibodies (see Cole, et al., In: Monoclonal Antibodies and Cancer Therapy, Alan R. Liss, Inc., 1985, pp. 77-96). If desired, human monoclonal antibodies may be prepared by using human hybridomas (see Cote, et al., Proc. Natl. Acad. Sci. USA 80:2026-2030, 1983) or by transforming human B-cells with Epstein Barr Virus in vitro (see Cole, et al., In: Monoclonal Antibodies and Cancer Therapy, supra). Each of the above citations are incorporated herein by reference in their entirety.

Techniques can be adapted for the production of single-chain antibodies specific to a GIP polypeptide, GIP-binding Groucho polypeptide (or fragment), Groucho-GIP chimeric polypeptide, or a complex of a GIP and Groucho polypeptide (see e.g., U.S. Pat. No. 4,946,778). In addition, methods can be adapted for the construction of Fab expression libraries (see e.g., Huse, et al., Science 246:1275-1281, 1989) to allow rapid and effective identification of monoclonal Fab fragments with the desired specificity for the desired protein or derivatives, fragments, analogs or homologs thereof. Non-human antibodies can be “humanized” by techniques well known in the art. See e.g., U.S. Pat. No. 5,225,539. Each of the above citations is incorporated herein by reference. Antibody fragments that contain the idiotypes to a GIP protein may be produced by techniques known in the art including, e.g.: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-GIP polypeptide, GIP binding Groucho polypeptide (or fragment), Groucho-GIP chimeric polypeptide, a complex of a GIP and Groucho polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al., Science 240:1041-1043, 1988; Liu et al., Proc. Nat. Acad. Sci. USA 84:3439-3443, 1987; Liu et al., J. Immunol. 139:3521-3526, 1987; Sun et al., Proc. Nat. Acad. Sci. USA 84:214-218, 1987; Nishimura et al., Cancer Res. 47:999-1005, 1987; Wood et al., Nature 314:446-449, 1985; Shaw et al., J. Natl. Cancer Inst. 80:1553-1559, 1988; Morrison, Science 229:1202-1207, 1985; Oi et al., BioTechniques 4:214, 1986; U.S. Pat. No. 5,225,539; Jones et al., Nature 321:552-525, 1986; Verhoeyan et al., Science 239:1534, 1988; and Beidler et al., J. Immunol. 141:4053-4060, 1988. Each of the above citations is incorporated herein by reference.

In one embodiment, methods for the screening of antibodies that possess the desired specificity include, e.g., enzyme-linked immunosorbent assay (ELISA) and other immunologically-mediated techniques known within the art. In a specific embodiment, selection of antibodies that are specific to a particular domain of a GIP polypeptide, GIP binding Groucho polypeptide (or fragment), Groucho-GIP chimeric polypeptide, or a complex of a GIP and Groucho polypeptide is facilitated by generation of hybridomas that bind to the fragment of a polypeptide or complex possessing such a domain. Antibodies that are specific for one or more domains within a given polypeptide or complex, e.g., the domain including the amino acids of SEQ ID NO: 7 in a GIP polypeptide, or derivatives, fragments, analogs or homologs thereof, are also provided herein.

An anti-GIP polypeptide, GIP binding Groucho polypeptide (or fragment), Groucho-GIP chimeric polypeptide, or a complex of a GIP and Groucho polypeptide antibodies may be used in methods known within the art relating to the localization and/or quantitation of a given polypeptide or complex (e.g., for use in measuring levels of the given polypeptide complex within appropriate physiological samples, for use in diagnostic methods, for use in imaging the protein, and the like). In one embodiment, antibodies for the given polypeptide or complex, or derivatives, fragments, analogs or homologs thereof, that contain the antibody derived binding domain, are utilized as pharmacologically-active compounds.

An anti-GIP polypeptide, GIP binding Groucho polypeptide (or fragment), Groucho-GIP chimeric polypeptide, or an anti-GIP-Groucho complex of a GIP and Groucho polypeptide antibody (e.g., monoclonal antibody) can be used to isolate GIP, Groucho polypeptides, or complexes including GIP polypeptides and Groucho polypeptides, by standard techniques, such as affinity chromatography or immunoprecipitation. Thus, the anti-antibodies disclosed herein can facilitate the purification of complexes of Groucho and GIP polypeptides from cells and of recombinantly produced Groucho and GIP polypeptides expressed in host cells.

An antibody of the invention can additionally be used to detect Groucho, GIP polypeptides, or complexes of GIP and Groucho polypeptides (e.g., in a cellular lysate or cell supernatant) in order to evaluate the abundance and pattern of expression of these polypeptides or complexes. Further, the herein disclosed antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Use of Groucho Protein-GIP Complexes or Their Component Polypeptides to Identify GIP and Groucho Interacting Agents

The GIP, Groucho, and complexes disclosed herein can also be used to identify compounds or other agents which modulate the activity of Groucho and/or GIP-mediated process. For example, to identify an agent that modulates Groucho or GIP activity, a GIP-Groucho complex is tested with a test agent and binding of the agent to the complex is measured. Binding of the agent to the complex indicates the agent modulates Groucho or GIP polypeptide activity.

Any compound or other molecule (or mixture or aggregate thereof) can be used as a test compound. In some embodiments, the agent can be a small peptide, or other small molecule produced by e.g., combinatorial synthetic methods known in the art. Binding of the compound to the complex can be determined using art recognized methods, e.g., by immunoprecipitation using antibodies (e.g., antibodies against GIP, Groucho, or the GIP-Groucho complex). Bound agents can be identified by comparing the relative electophoretic mobility of complexes exposed to the test agent to the mobility of complexes that have not been exposed to the test agent. Altered migration of the test complexes indicates the test agent binds to the GIP-Groucho complex.

Also provided for in the invention is a method for identifying agents which modulate GIP activity by contacting a GIP polypeptide (or a GIP-binding fragment of a Groucho polypeptide) with a test agent, and measuring binding of the agent to the complex. Binding of the agent to the complex indicates the agent modulates GIP polypeptide activity. As described above, any art-recognized method for determining binding of a compound to a test compound can be used.

Agents identified in the screening assays can be further tested for their ability to alter and/or modulate cellular functions, particularly those functions in which the Groucho protein has been implicated. These functions include, e.g., regulation of transcription and the fate of differentiation, as well as various other biological activities (e.g., binding to an anti-Groucho protein●GIP complex antibody).

Methods ofDiagnosing Conditions Associated with Altered Levels of GIP Polypeptides or Groucho-GIP Polypeptide Complexes

Groucho is implicated in multiple biological processes, and, as described herein, the fate of cell differentiation. Accordingly, a variety of conditions can be or identified in subjects by measuring the levels of Groucho-GIP complexes in a subject and comparing the levels of the Groucho-GIP complexes to the levels of the complexes in a reference population whose corresponding status with respect to the compared condition is known.

Comparable levels of the Groucho-GIP complex in the test sample and the reference sample indicates the subject has the Groucho-GIP complex associated disorder (or absence thereof) as that of the reference population. In contrast, altered levels of the complex in the test and reference populations indicates the subject's status with respect to the Groucho-GIP associated disorder is different from that in the control population. Thus, if the reference cell population includes cells from individuals that do not have the Groucho-GIP associated disorder, a similarity in GIP-Groucho complex levels in the test and control populations indicates the subject does not have the GIP-Groucho complex-associated disorder. Conversely, a difference in the levels of the test and reference populations indicates the subject has, or has a predisposition to, the Groucho-GIP disorder.

In general, a test cell population from the subject includes at least one cell that is capable of expressing genes encoding the polypeptide, or polypeptides making up the measured complex (i.e., the cell expresses a GIP polypeptide, Groucho, polypeptide, or both). By “capable of expressing” is meant that the corresponding gene or genes is present in an intact form in the cell and can be expressed.

In general, any reference cell population can be used, as long as its status with respect to the measure parameter is known (i.e., the reference cell population is known to possess or lack the property or trait being measured in the test cell population), and whose level of GIP-Groucho complex is known. In some embodiments, the reference cell population is made up substantially, or preferably exclusively, of such cells whose status is known.

Preferably, cells in the reference cell population are derived from a tissue type as similar as possible to a test cell. In some embodiments, the control cell is derived from the same subject as the test cell. In other embodiments, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

In some embodiments, the reference cell population is derived from a plurality of cells. For example, the reference cell population can be a database of expression patterns from previously tested cells for which one of the herein-described parameters or conditions is known.

If desired, comparison of differentially expressed sequences between a test cell population and a reference cell population can be done with respect to a expression of a control gene whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter. Thus, a test cell population may be compared to a second reference cell population known to contain, e.g., tumorous cells, as well as a second reference population known to contain, e.g., non-tumorous cells.

The test cell or cell population in any of the herein described diagnostic or screening assays can be taken from a known or suspected tumor containing sample or from a bodily fluid, e.g., biological fluid (such as blood, serum, urine, saliva, milk, ductal fluid, or tears). For many applications, cells present in a bodily fluid can be examined instead of a primary lesion. Thus, the need for taking a biopsy from a known or suspected primary tumor site is obviated.

In another aspect, disorders associated with altered levels of GIP or Groucho-GIP complexes are identified in a subject by measuring expression of GIP nucleic acids. Expression of GIP sequences can be detected (if present) and measured using techniques well known to one of ordinary skill in the art. For example, GIP sequences, including those disclosed herein (e.g. SEQ ID NO: 12) can be used to construct probes for detecting GIP RNA sequences in, e.g., northern blot hybridization analyses. As another example, the sequences can be used to construct primers for specifically amplifying sequences in, e.g., amplification-based detection methods such as reverse-transcription based polymerase chain reaction.

Expression level of GIP sequences in the test cell population is then compared to expression levels of the sequences in one or more cells from a reference cell population.

Expression of the genes disclosed herein can be measured at the RNA level using any method known in the art. For example, northern hybridization analysis using probes which specifically recognize one or more of these sequences can be used to determine gene expression. Alternatively, expression can be measured using reverse-transcription-based PCR assays, e.g., using primers specific for the differentially expressed sequences. When alterations in gene expression are associated with gene amplification or deletion, sequence comparisons in test and reference populations can be made by comparing relative amounts of the examined DNA sequences in the test and reference cell populations.

Levels of Groucho protein-GIP complexes can be used to determine the presence of, or predisposition to, multiple states in a subject. For example, these complexes can serve as markers for specific disease states that involve the disruption of physiological processes. These processes can include e.g., control of cellular differentiation or regulation of transcription. In addition, a subject can be assessed for the presence, or predisposition to, pathological processes. These processes can include, e.g., hyperproliferative disorders (e.g., tumorigenesis and tumor progression).

In addition to diagnostic methods, the herein described methods can be used to assess the prognosis, or follow the course of, a disease or condition associated with altered levels of GIP-Groucho complexes (or its components) in a subject. These methods can additionally be used to determine the efficacy of administered therapeutics.

To detect GIP-Groucho complexes, the herein disclosed antibodies may be used. For example, anti GIP-Groucho complex antibodies or anti-GIP antibodies can be used in assays (e.g., immunoassays) to detect, prognose, diagnose, or monitor various conditions, diseases, and disorders characterized by aberrant levels of Groucho protein-GIP complex. They can alternatively used to monitor the treatment of conditions associated with altered levels of these complexes, or their components thereof.

To perform immunoassays using antibodies for GIP-Groucho complexes, a sample derived from a patient is contacted with an anti-Groucho protein-GIP complex antibody under conditions such that specific binding may occur. Specific binding by the antibody is then detected, if present, and quantitated.

In a specific embodiment, an antibody specific for a Groucho protein-GIP complex is used to analyze a tissue or serum sample from a patient for the presence of Groucho protein-GIP complex. An aberrant level of Groucho protein-GIP complex is indicative of a diseased condition. The immunoassays which may be utilized include, e.g., competitive and non-competitive assay systems using techniques such as Western Blots, radioimmunoassays (RIA), enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein-A immunoassays.

In some embodiments, diseases and disorders involving or characterized by aberrant levels of Groucho protein-GIP complex or a predisposition to develop such disorders may be diagnosed by detecting aberrant levels of Groucho protein-GIP complex, or non-complexed Groucho protein and/or GIP proteins or nucleic acids for functional activity. Suitable functional activities that can be assayed include, e.g.,(i) binding to an interacting partner (e.g., the Groucho protein, GIP) or (ii) by detecting mutations in Groucho protein and/or a GIP RNA, DNA or protein, (e.g., translocations, truncations, changes in nucleotide or amino acid sequence relative to wild-type Groucho protein and/or the GIP) which can cause increased or decreased expression or activity of the Groucho protein, a GIP or a Groucho protein-GIP complex.

Methods which are well-known within the art (e.g., immunoassays, nucleic acid hybridization assays, biological activity assays, and the like) may be used to determine whether one or more particular Groucho protein-GIP complexes are present at either increased or decreased levels, or are absent, within samples derived from patients suffering from a particular disease or disorder, or possessing a predisposition to develop such a disease or disorder, as compared to the levels in samples from subjects not having such disease or disorder or predisposition thereto. Additionally, these assays may be utilized to determine whether the ratio of the Groucho protein-GIP complex to the non-complexed components (i.e. the Groucho protein and/or the specific GIP) in the complex of interest is increased or decreased in samples from patients suffering from a particular disease or disorder or having a predisposition to develop such a disease or disorder as compared to the ratio in samples from subjects not having such a disease or disorder or predisposition thereto.

Accordingly, in specific embodiments of the present invention, diseases and disorders which involve increased/decreased levels of one or more Groucho protein-GIP complex may be diagnosed, or their suspected presence may be screened for, or a predisposition to develop such diseases and disorders may be detected, by quantitatively ascertaining increased/decreased levels of: (i) the one or more Groucho protein-GIP complex; (ii) the mRNA encoding both protein members of the complex; (iii) the complex functional activity or (iv) mutations in the Groucho protein or the GIP (e.g., translocations in nucleic acids, truncations in the gene or protein, changes in nucleotide or amino acid sequence relative to wild-type Groucho protein or the GIP) which enhance/inhibit or stabilize/destabilize Groucho protein-GIP complex formation.

Antibodies directed against the Groucho protein-GIP complex can also be used to detect cells which express the protein or protein complexes. Using such assays, specific cell types may be quantitatively characterized in which one or more particular Groucho protein-GIP complex are expressed, and the presence of the component polypeptides or protein complex may be correlated with cell viability by techniques well-known within the art (e.g., florescence-activated cell sorting).

In some embodiments, expression is detected in in vitro cell culture models which express particular Groucho protein-GIP complex, or derivatives thereof, for the purpose of characterizing and/or isolating Groucho protein-GIP complex. These detection techniques include, e.g.,, cell-sorting of prokaryotes (see e.g., Davey & Kell, 1996. Microbiol. Rev. 60:641-696); primary cultures and tissue specimens from eukaryotes, including mammalian species such as human (see e.g., Steele, et al., 1996. Clin. Obstet. Gynecol. 39:801-813) and continuous cell cultures (see e.g., Orfao & Ruiz-Arguelles, 1996. Clin. Biochem. 29:5-9.

The observation that GIP interacts with Groucho polypeptide can also be used to detect the presence of, and, if desired, to purify, the binding polypeptides in a biological sample. For example, levels of Groucho polypeptide in a biological sample can also be measured by contacting the sample with a labeled polypeptide including the Groucho-binding domain of a GIP polypeptide. Similarly, the presence of GIP in a biological sample can be measured by be measured by contacting the sample with a polypeptide that includes a GIP binding region or domain of a Groucho polypeptide.

Kits Containing Reagents for Identifying GIP-Groucho Complexes

The invention additionally provides kits for diagnostic use. The kits include one or more containers containing an anti-Groucho protein-GIP complex antibody and, optionally, a labeled binding partner to the antibody. The label incorporated into the anti-Groucho protein-GIP complex antibody may include, e.g., a chemiluminescent, enzymatic, fluorescent, calorimetric or radioactive moiety. Alternatively, the kit may include, in one or more containers, a pair of oligonucleotide primers (e.g., each 6-30 nucleotides in length) which are capable of acting as amplification primers for: polymerase chain reaction (PCR; see e.g., Innis, et al., 1990. PCR Protocols, Academic Press, Inc., San Diego, Calif.), ligase chain reaction, cyclic probe reaction, or other methods known within the art. The kit may, optionally, further comprise a predetermined amount of a purified Groucho protein, GIP or Groucho-GIP complex, or nucleic acids thereof, for use as a standard or control in the aforementioned assays.

Transgenic Animals

The host cells of the invention can also be used to produce nonhuman transgenic animals. For example, in one embodiment, a host cell of the invention is a fertilized oocyte or an embryonic stem cell into which GIP-coding sequences have been introduced. Such host cells can then be used to create non-human transgenic animals in which exogenous GIP sequences have been introduced into their genome or homologous recombinant animals in which endogenous GIP sequences have been altered. Such animals are useful for studying the function and/or activity of GIP and for identifying and/or evaluating modulators of GIP activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GIP gene has been altered by homologous recombination between the endogenous gene and an exogenous DNA molecule introduced into a cell of the animal, e.g., an embryonic cell of the animal, prior to development of the animal.

A transgenic animal of the invention can be created by introducing GIP-encoding nucleic acid into the male pronuclei of a fertilized oocyte, e.g., by microinjection, retroviral infection, and allowing the oocyte to develop in a pseudopregnant female foster animal. The huma GIP DNA sequence of SEQ ID NO: 12 can be introduced as a transgene into the genome of a non-human animal. Alternatively, a nonhuman homologue of the huma GIP gene, such as a mouse GIP gene, can be isolated based on hybridization to the huma GIP cDNA (described further above) and used as a transgene. Intronic sequences and polyadenylation signals can also be included in the transgene to increase the efficiency of expression of the transgene. A tissue-specific regulatory sequence(s) can be operably linked to the GIP transgene to direct expression of GIP protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan In: Manipulating the Mouse Embryo, Cold Spring Harbor Laboratory Press, Cold Spring Harbor, N.Y, 1986. Similar methods are used for production of other transgenic animals. A transgenic founder animal can be identified based upon the presence of the GIP transgene in its genome and/or expression of GIP mRNA in tissues or cells of the animals. A transgenic founder animal can then be used to breed additional animals carrying the transgene. Moreover, transgenic animals carrying a transgene encoding GIP can further be bred to other transgenic animals carrying other transgenes.

To create a homologous recombinant animal, a vector is prepared which contains at least a portion of a GIP gene into which a deletion, addition or substitution has been introduced to thereby alter, e.g., functionally disrupt, the GIP gene. The GIP gene can be a human gene, but more preferably, is a non-human homologue of a huma GIP gene. For example, a mouse homologue of a huma GIP gene can be used to construct a homologous recombination vector suitable for altering an endogenous GIP gene in the mouse genome. In one embodiment, the vector is designed such that, upon homologous recombination, the endogenous GIP gene is functionally disrupted (ie., no longer encodes a functional protein; also referred to as a “knock out” vector).

Alternatively, the vector can be designed such that, upon homologous recombination, the endogenous GIP gene is mutated or otherwise altered but still encodes functional protein (e.g., the upstream regulatory region can be altered to thereby alter the expression of the endogenous GIP protein). In the homologous recombination vector, the altered portion of the GIP gene is flanked at its 5′ and 3′ ends by additional nucleic acid of the GIP gene to allow for homologous recombination to occur between the exogenous GIP gene carried by the vector and an endogenous GIP gene in an embryonic stem cell. The additional flanking GIP nucleic acid is of sufficient length for successful homologous recombination with the endogenous gene. Typically, several kilobases of flanking DNA (both at the 5′ and 3′ ends) are included in the vector. See e.g., Thomas et al., Cell 51:503, 1987, for a description of homologous recombination vectors. The vector is introduced into an embryonic stem cell line (e.g., by electroporation) and cells in which the introduced GIP gene has homologously recombined with the endogenous GIP gene are selected (see e.g., Li et al., Cell 69:915, 1992).

The selected cells are then injected into a blastocyst of an animal (e.g., a mouse) to form aggregation chimeras. See e.g., Bradley, In: Teratocarcinomas and Embryonic Stem Cells: A Practical Approach, Robertson, ed. IRL, Oxford, 1987, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, Curr. Opin. Biotechnol. 2:823-829, 1991; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al., Proc. Nat. Acad. Sci. USA 89:6232-6236, 1992. Another example of a recombinase system is the FLP recombinase system of Saccharomyces cerevisiae (O'Gorman et al., Science 251:1351-1355, 1991. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., Nature 385:810-813, 1997. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Alternatively, transgenic animals can be prepared as above using nucleotide sequences encoding a GIP-binding fragment of a Groucho protein or a Groucho protein-GIP chimeric polypeptide.

Therapeutic Uses of GIP Polypeptides, GIP Binding Groucho Polypeptides, and Groucho Protein-GIP Complexes

The Groucho protein plays a significant role in cell differentiation and transcriptional control.

Based in part on the discovery of the GIP-Groucho interaction, the invention provides for treatment or prevention of various diseases and disorders by administration of a biologically-active, therapeutic compound (hereinafter “Therapeutic”). Such Therapeutics include, e.g.,: (i) various Groucho protein.GIP complexes (e.g., the Groucho protein complexed with GIP) and derivative, fragments, analogs and homologs thereof; (ii) antibodies directed against these proteins and protein complexes; (iii) nucleic acids encoding the Groucho protein and GIP and derivatives, fragments, analogs and homologs thereof; (iv) antisense nucleic acids encoding the Groucho protein and (v) Groucho protein IPs and Groucho protein.GIP complex and modulators (i.e., inhibitors, agonists and antagonists) thereof.

(i) Disorders with Increased Groucho Protein and Groucho Protein.GIP Complex Levels

Diseases and disorders which are characterized by increased (relative to a subject not suffering from the disease or disorder) Groucho protein-GIP levels or biological activity may be treated with Therapeutics which antagonize (i.e., reduce or inhibit) Groucho protein-GIP complex formation or activity. Therapeutics which antagonize Groucho protein-GIP complex formation or activity may be administered in a therapeutic or prophylactic manner. Therapeutics which may be utilized include, e.g.,, the Groucho protein or GIP, or analogs, derivatives, fragments or homologs thereof; (ii) anti-Groucho protein-GIP complex antibodies; (iii) nucleic acids encoding the Groucho protein or GIP; (iv) concurrent administration of a Groucho protein and a GIP antisense nucleic acid and Groucho protein and/or GIP nucleic acids which are “dysfunctional” (ie., due to a heterologous [non-Groucho protein and/or non-GIP] insertion within the coding sequences of the Groucho protein and GIP coding sequences) are utilized to “knockout” endogenous Groucho protein and/or GIP function by homologous recombination (see e.g., Capecchi, Science 244:1288-1292, 1989). Alternatively, mutants or derivatives of a first GIP which possess greater affinity for Groucho protein than the wild-type first GIP may be administered to compete with a second GIP for binding to the Groucho protein, thereby reducing the levels of complex between the Groucho protein and the second GIP.

Increased levels of Groucho protein-GIP complex can be readily detected by quantifying protein and/or RNA, by obtaining a patient tissue sample (e.g., from biopsy tissue) and assaying it in vitro for RNA or protein levels, structure and/or activity of the expressed Groucho protein-GIP complex (or the Groucho protein and GIP mRNAs). Methods which are well-known within the art including, e.g., immunoassays to detect Groucho protein-GIP complex (e.g., by Western blot analysis, immunoprecipitation followed by sodium dodecyl sulfate (SDS) polyacrylamide gel electrophoresis, immunocytochemistry, etc.) and/or hybridization assays to detect concurrent expression of the Groucho protein and GIP mRNAs (e.g., Northern assays, dot blots, in situ hybridization, etc.).

(ii) Disorders with Increased Groucho Protein and Groucho Protein-GIP Complex Levels

The invention includes methods for the reduction of Groucho protein-GIP complex expression (i.e., the expression of the two protein components of the complex and/or formation of the complex) by targeting mRNAs which express the protein moieties. RNA Therapeutics are differentiated into three classes: (i) antisense species; (ii) ribozymes or (iii) RNA aptamers. See e.g., Good, et al., 1997. Gene Therapy 4:45-54. Antisense therapy will be discussed below. Ribozyme therapy involves the administration (i.e., induced expression) of small RNA molecules with enzymatic ability to cleave, bind, or otherwise inactivate specific RNAs, thus reducing or eliminating the expression of particular proteins. See e.g., Grassi & Marini, 1996. Ann. Med. 28:499-510. RNA aptamers are specific RNA ligands for proteins, such as for Tat and Rev RNA (see e.g., Good, et al., 1997. Gene Therapy 4:45-54) which can specifically inhibit their translation.

In one embodiment, the activity or level of the Groucho protein may be reduced by administration of GIP, a nucleic acid which encodes GIP or an antibody (or a derivative or fragment of the antibody possessing the binding domain thereof) which specifically binds to GIP. Similarly, the levels or activity of GIP may be reduced by administration of the Groucho protein, a nucleic acid encoding the Groucho protein or an antibody (or a derivative or fragment of the antibody possessing the binding domain thereof) which specifically binds the Groucho protein. In another embodiment of the present invention, diseases or disorders which are associated with increased levels of the Groucho protein or GIP, may be treated or prevented by administration of a Therapeutic which increases Groucho protein-GIP complex formation, if the complex formation acts to reduce or inactivate the Groucho protein or the particular GIP via Groucho protein-GIP complex formation. Such diseases or disorders may be treated or prevented by: (i) the administration of one member of the Groucho protein-GIP complex, including mutants of one or both of the proteins which possess increased affinity for the other member of the Groucho protein-GIP complex (so as to cause increased complex formation) or (ii) the administration of antibodies or other molecules which serve to stabilize the Groucho protein-GIP complex, or the like.

Determination of the Biological Effect of the Therapeutic

In some embodiments, suitable in vitro or in vivo assays are utilized to determine the effect of a specific Therapeutic and whether its administration is indicated for treatment of the affected tissue.

For example, in vitro assays may be performed with representative cells of the type(s) involved in the patient's disorder, to determine if a given Therapeutic exerts the desired effect upon the cell type(s). Compounds for use in therapy may be tested in suitable animal model systems including, e.g. rats, mice, chicken, cows, monkeys, rabbits, and the like, prior to testing in human subjects. Similarly, for in vivo testing, any of the animal model system known in the art may be used prior to administration to human subjects.

(i) Malignancies

Components of the Groucho protein-GIP complex may be involved in the regulation of cell proliferation. Accordingly, Therapeutics of the present invention may be useful in the therapeutic or prophylactic treatment of diseases or disorders which are associated with cell hyperproliferation and/or loss of control of cell proliferation (e.g., cancers, malignancies and tumors). For a review of such hyperproliferation disorders, see e.g., Fishman, et al., 1985. Medicine, 2nd ed (J.B. Lippincott Co., Philadelphia, Pa.).

Therapeutics of the present invention may be assayed by any method known within the art for efficacy in treating or preventing malignancies and related disorders. Such assays include, e.g.,, in vitro assays utilizing transformed cells or cells derived from the patient's tumor, as well as in vivo assays using animal models of cancer or malignancies. Potentially effective Therapeutics, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) Groucho protein-GIP complex activity, that cancer or malignancy may subsequently be treated or prevented by the administration of a Therapeutic which serves to modulate Groucho protein-GIP complex formation and function, including supplying Groucho protein-GIP complex and the individual binding partners of the protein complex (i.e., the Groucho protein and/or GIP).

(ii) Pre-Malignant Conditions

The Therapeutics of the present invention which are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. Basic Pathology, 2nd ed. (W.B. Saunders Co., Philadelphia, Pa.).

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic of the present invention which possesses the ability to modulate Groucho protein-GIP complex activity. Characteristics of a transformed phenotype include, e.g.,: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 Kdal cell-surface protein, and the like. See e.g., Richards, et al., 1986. Molecular Pathology (W.B. Saunders Co., Philadelphia, Pa.).

In one embodiment, a patient which exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myelogenous leukemia and t(14;18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

(iii) Hyperproliferative and Dysproliferative Disorders

In a preferred embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Once a hyperproliferative disorder has been shown to be amenable to treatment by modulation of Groucho protein-GIP complex activity, the hyperproliferative disease or disorder may be treated or prevented by the administration of a Therapeutic which modulates Groucho protein●GIP complex formation (including supplying Groucho protein●GIP complex and the individual binding partners of a Groucho proteine●GIP complex.

In some embodiments, methods are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Gene Therapy Using GIP and/or Groucho Nucleic Acids

In one embodiment, nucleic acids comprising a sequence which encodes the Groucho protein and/or GIP, or functional derivatives thereof, are administered to modulate Groucho protein●GIP complex function, using gene therapy, i.e., a nucleic acid or nucleic acids encoding both the Groucho protein and GIP, or functional derivatives thereof, are administered to a subject. After delivery of the nucleic acid to a subject, the nucleic acid expresses its encoded protein(s), which then serve to exert a therapeutic effect by modulating Groucho protein●GIP complex function. Any of the methods relating to gene therapy available within the art may be used in the practice of the present invention. See, e.g., Goldspiel, et al.,. Clin. Pharm. 12:488-505, 1993, and U.S. Pat. No. 5,580,859.

In one embodiment, the Therapeutic includes a nucleic acid encoding a Groucho protein and GIP nucleic acid which is part of an expression vector expressing both proteins, or fragments or chimeric proteins thereof, within a suitable host. In a specific embodiment, such a nucleic acid possesses a promoter which is operably-linked to the Groucho protein and the GIP coding region(s), or, less preferably two separate promoters linked to the Groucho protein and the GIP coding regions separately; wherein the promoter is inducible or constitutive, and, optionally, tissue-specific. In another specific embodiment, a nucleic acid molecule is used in which the Groucho protein and GIP coding sequences (and any other desired sequences) are flanked by regions which promote homologous recombination at a desired site within the genome, thus providing for intra-chromosomal expression of the Groucho protein and the GIP nucleic acids. See e.g., Koller & Smithies, 1989. Proc. Natl. Acad. Sci. USA 86:8932-8935.

Delivery of the Therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. For example, the nucleic acid can be directly administered in vivo, where it is expressed to produce the encoded product. This may be accomplished by any of numerous methods known in the art including, e.g.: (i) constructing it as part of an appropriate nucleic acid expression vector and administering in a manner such that it becomes intracellular (e.g., by infection using a defective or attenuated retroviral or other viral vector; see U.S. Pat. No. 4,980,286) or (ii) direct injection of naked DNA, or through the use of microparticle bombardment (e.g., a “Gene Gun®; Biolistic, Dupont), or by coating it with lipids, cell-surface receptors/transfecting agents, or through encapsulation in liposomes, microparticles, or microcapsules, or by administering it in linkage to a peptide which is known to enter the nucleus, or by administering it in linkage to a ligand predisposed to receptor-mediated endocytosis (see e.g., Wu & Wu, 1987. J. Biol. Chem. 262:4429-4432), which can be used to “target” cell types which specifically express the receptors of interest, etc.

In another specific embodiment of the present invention, a nucleic acid-ligand complex may be produced in which the ligand comprises a fusogenic viral peptide designed so as to disrupt endosomes, thus allowing the nucleic acid to avoid subsequent lysosomal degradation. In yet another specific embodiment, the nucleic acid may be targeted in vivo for cell-specific endocytosis and expression, by targeting a specific receptor. See e.g., PCT Publications WO 92/06180; W093/14188 and WO 93/20221. Alternatively, the nucleic acid may be introduced intracellularly and incorporated within host cell genome for expression by homologous recombination. See e.g., Zijlstra, et al., 1989. Nature 342:435-438.

In yet another embodiment, a viral vector which contains the Groucho protein and/or GIP nucleic acids is utilized. For example, retroviral vectors may be employed (see e.g., Miller, et al., 1993. Meth. Enzymol. 217:581-599) which have been modified to delete those retroviral-specific sequences which are not required for packaging of the viral genome and its subsequent integration into host cell DNA. The Groucho protein and/or GIP (preferably both protein species) nucleic acids are cloned into the vector, which facilitates delivery of the genes into a patient. See e.g., Boesen, et al., 1994. Biotherapy 6:291-302; Kiem, et al., 1994. Blood 83:1467-1473. Additionally, adenovirus is an especially efficacious “vehicle” for the delivery of genes to the respiratory epithelia. Other targets for adenovirus-based delivery systems are liver, the central nervous system, endothelial cells, and muscle. Adenoviruses also possess the advantageous ability to infect non-dividing cells. For a review see e.g., Kozarsky & Wilson, 1993. Curr. Opin. Gen. Develop. 3:499-503. Adenovirus-associated virus has also been proposed for use in gene therapy. See e.g., Walsh, et al., 1993. Proc. Soc. Exp. Biol. Med. 204:289-300.

An additional approach to gene therapy in the practice of the present invention involves transferring a gene into cells in in vitro tissue culture by such methods as electroporation, lipofection, calcium phosphate-mediated transfection, or viral infection. Generally, the method of transfer includes the transfer of a selectable marker to the cells. The cells are then placed under selection pressure (e.g., antibiotic resistance) so as facilitate the isolation of those cells which have taken up, and are expressing the transferred gene. Those cells are then delivered to a patient. In this specific embodiment, the nucleic acid is introduced into a cell prior to the in vivo administration of the resulting recombinant cell by any method known within the art including, e.g.: transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methods which ensure that the necessary developmental and physiological functions of the recipient cells are not disrupted by the transfer. See e.g., Loeffler & Behr, 1993. Meth. Enzymol. 217: 599-618. The technique should provide for the stable transfer of the nucleic acid to the cell, so that the nucleic acid is expressible by the cell and preferably heritable and expressible by its cell progeny.

The resulting recombinant cells may be delivered to a patient by various methods known within the art including, e.g.: injection of epithelial cells (e.g., subcutaneously); the application of recombinant skin cells as a skin graft onto the patient and the intravenous injection of recombinant blood cells (e.g., hematopoetic stem or progenitor cells). The total amount of cells which are envisioned for use depend upon the desired effect, patient state, etc., and may be determined by one skilled within the art.

Cells into which a nucleic acid can be introduced for purposes of gene therapy encompass any desired, available cell type, and include e.g., epithelial cells, endothelial cells, keratinocytes, fibroblasts, muscle cells, hepatocytes and blood cells. In a preferred embodiment of the present invention, the cell utilized for gene therapy may be autologous to the patient.

In a specific embodiment in which recombinant cells are used in gene therapy, stem or progenitor cells, which can be isolated and maintained in vitro, may be utilized. Such stem cells include, e.g.,, hematopoetic stem cells (HSC), stem cells of epithelial tissues and neural stem cells (see e.g., Stemple & Anderson, 1992. Cell 71:973-985). Any technique which provides for the isolation, propagation, and maintenance in vitro of HSC may be used. HSCs utilized for gene therapy are, preferably, autologous to the patient. Hence, non-autologous HSCs are, preferably, utilized in conjunction with a method of suppressing transplantation immune reactions of the future host/patient. See e.g., Kodo, et al., 1984. J. Clin. Invest. 73:1377-1384. In another embodiment, HSCs may be highly enriched (or produced in a substantially-pure form), by any techniques known within the art, prior to administration to the patient. See e.g., Witlock & Witte, 1982. Proc. Natl. Acad. Sci. USA 79:3608-3612.

Anti-Sense GIP or Groucho Oligonucleotides

Groucho protein-GIP complex formation and function may be inhibited by the use of anti-sense nucleic acids for the Groucho protein and/or GIP. In some embodiments, nucleic acids (of at least six nucleotides in length) which are anti-sense to a genomic sequence (gene) or cDNA encoding the Groucho protein and/or GIP, or portions thereof, are used prophylactically or therapeutically. Such anti-sense nucleic acids have utility as Therapeutics which inhibit Groucho protein.GIP complex formation or activity, and may be utilized in a therapeutic or prophylactic manner.

The invention also provides methods for inhibiting expression of the Groucho protein and GIP nucleic acid sequences within a prokaryotic or eukaryotic cell. The method includes providing the cell with an therapeutically-effective amount of an anti-sense nucleic acid of the Groucho protein and GIP, or derivatives thereof.

The anti-sense nucleic acids may be oligonucleotides which may either be directly administered to a cell or which may be produced in vivo by transcription of the exogenous, introduced sequences. In addition, the anti-sense nucleic acid may be complementary to either a coding (i.e., exonic) and/or non-coding (i.e., intronic) region of the Groucho protein or GIP mRNAs. The Groucho protein and GIP anti-sense nucleic acids are, at least, six nucleotides in length and are, preferably, oligonucleotides ranging from 6-200 nucleotides in length. In specific embodiments, the anti-sense oligonucleotide is at least 10 nucleotides, at least 15 nucleotides, at least 100 nucleotides, or at least 200 nucleotides. The anti-sense oligonucleotides may be DNA or RNA (or chimeric mixtures, derivatives or modified versions thereof), may be either single-stranded or double-stranded and may be modified at a base, sugar or phosphate backbone moiety.

In addition, the anti-sense oligonucleotide may include other associated functional groups, such as peptides, moieties which facilitate the transport of the oligonucleotide across the cell membrane, a hybridization-triggered cross-linking agent, a hybridization-triggered cleavage-agent, and the like. See e.g., Letsinger, et al., 1989. Proc. Natl. Acad. Sci. U.S.A. 86:6553-6556; PCT Publication No. WO 88/09810. In a specific embodiment, the Groucho protein and GIP antisense oligonucleotides include catalytic RNAs or ribozymes. See, e.g., Sarver, et al., 1990. Science 247:1222-1225.

The anti-sense oligonucleotides may be synthesized by standard methods known within the art including, e.g.: (i) automated phosphorothioate-mediated oligonucleotide synthesis (see e.g., Stein, et al., 1988. Nuc. Acids Res. 16:3209) or (ii) methylphosphonate oligonucleotides can be prepared by use of controlled pore glass polymer supports (see e.g., Sarin, et al., 1988. Proc. Natl. Acad. Sci. U.S.A. 85:7448-7451).

In an alternative embodiment, the Groucho protein and GIP antisense nucleic acids are produced intracellularly by transcription of an exogenous sequence. For example, a vector may be produced and (upon being exocytosed by the cell) transcribed in vivo, thus producing an antisense nucleic acid (RNA) species. The vector may either remain episomal or become chromosomally-integrated, so long as it can be transcribed to produce the desired antisense RNA. The vectors may be derived from bacterial, viral, yeast or other sources known within the art, which are utilized for replication and expression in mammalian cells. Expression of the sequences encoding the Groucho protein and GIP antisense RNAs may be facilitated by any promoter known within the art to function in mammalian, preferably, human cells. Such promoters may be inducible or constitutive and include, e.g.,: (i) the SV40 early promoter region; (ii) the promoter contained in the 3′-terminus long terminal repeat of Rous sarcoma virus (RSV); (iii) the Herpesvirus thymidine kinase promoter and (iv) the regulatory sequences of the metallothionein gene.

The Groucho protein and GIP antisense nucleic acids may be utilized prophylactically or therapeutically in the treatment or prevention of disorders of a cell type which expresses (or over-expresses) the Groucho protein-GIP complex. Cell types which express or over-express the Groucho protein and GIP RNA may be identified by various methods known within the art including, e.g.,, hybridization with Groucho protein- and GIP-specific nucleic acids (e.g., by Northern hybridization, dot blot hybridization, in situ hybridization) or by observing the ability of RNA from the specific cell type to be translated in vitro into the Groucho protein and the GIP by immunohistochemistry. If desired, primary tissue from a patient may be assayed for the Groucho protein and/or GIP expression prior to actual treatment by, for example, immunocytochemistry or in situ hybridization.

Pharmaceutical compositions which include an effective amount of a Groucho protein and GIP antisense nucleic acid contained within a pharmaceutically-acceptable carrier may be administered to a patient having a disease or disorder which is of a type that expresses or over-expresses Groucho protein-GIP complex RNA or protein. The amount of Groucho protein and/or GIP antisense nucleic acid which will be effective in the treatment of a particular disorder or condition will be dependant upon the nature of the disorder or condition, and may be determined by standard clinical techniques. Where possible, it is desirable to determine the antisense cytotoxicity in vitro, and then in useful animal model systems prior to testing and use in humans. In a specific embodiment, pharmaceutical compositions comprising Groucho protein and GIP antisense nucleic acids may be administered via liposomes, microparticles, or microcapsules. See e.g., Leonetti, et al., 1990. Proc. Natl. Acad. Sci. U.S.A. 87:2448-2451.

Groucho Protein-GIP Complex Assays

The functional activity of Groucho protein-GIP complexes (and derivatives, fragments, analogs and homologs thereof) may be assayed by a number of methods known within the art. For example, putative modulators (e.g., inhibitors, agonists and antagonists) of Groucho protein-Groucho protein complex activity (e.g., anti-Groucho protein-GIP complex antibodies, as well as Groucho protein or GIP antisense nucleic acids) may be assayed for their ability to modulate Groucho protein-GIP complex formation and/or activity.

(i) Immunoassays

Also disclosed herein are immunoassay-based useful for measuring the ability of an altered complex, e.g. a complex containing derivatives, fragments, analogs and/or homologs thereof of a Groucho or GIP polypeptide to bind to, or compete with, wild-type Groucho protein-GIP complex or GIP. Alternatively, immunoassays be used to determine the ability of the altered complex to bind to an anti-Groucho protein-GIP complex antibody. These immunoassays include, e.g.,, competitive and non-competitive assay systems utilizing techniques such as radioimmunoassays, enzyme linked immunosorbent assay (ELISA), “sandwich” immunoassays, immunoradiometric assays, gel diffusion precipitin reactions, immunodiffusion assays, in situ immunoassays (e.g., using colloidal gold, enzyme or radioisotope labels), Western blots, Northwestern blots, precipitation reactions, agglutination assays (e.g., gel agglutination assays, hemagglutination assays), complement fixation assays, immunofluorescence assays, protein-A assays and immunoelectrophoresis assays, and the like. In one specific embodiment of the present invention, antibody binding is detected by assaying for a label on the primary antibody. In another specific embodiment, the binding of the primary antibody is ascertained by the detection of the binding of a secondary antibody (or reagent) specific for the primary antibody. In a further embodiment, the secondary antibody is labeled.

(ii) Gene Expression Assays

The expression of the Groucho protein or GIP genes (both endogenous genes and those expressed from recombinant DNA) may be detected using techniques known within the art including, e.g.: Southern hybridization, Northern hybridization, restriction endonuclease mapping, DNA sequence analysis and polymerase chain reaction amplification (PCR) followed by Southern hybridization or RNase protection (see e.g., Current Protocols in Molecular Biology 1997. (John Wiley and Sons, New York, N.Y.)) with probes specific for the Groucho protein and GIP genes in various cell types.

In one specific embodiment of the present invention, Southern hybridization may be used to detect genetic linkage of the Groucho protein and/or GIP gene mutations to physiological or pathological states. Numerous cell types, at various stages of development, may be characterized for their expression of the Groucho protein and GIP (particularly the concomitant expression of the Groucho protein and GIP within the same cells). The stringency of the hybridization conditions for Northern or Southern blot analysis may be manipulated to ensure detection of nucleic acids with the desired degree of relatedness to the specific probes used. Modification of these aforementioned methods, as well as other methods well-known within the art, may be utilized in the practice of the present invention.

(iii) Binding Assays

Derivatives, fragments, analogs and homologs of GIP may be assayed for binding to the Groucho protein by any method known within the art including, e.g.: (i) the modified yeast two hybrid assay system; (ii) immunoprecipitation with an antibody which binds to the Groucho protein within a complex, followed by analysis by size fractionation of the immunoprecipitated proteins (e.g., by denaturing or non-denaturing polyacrylamide gel electrophoresis); (iii) Western analysis; (v) non-denaturing gel electrophoresis, and the like.

(iv) Assays for Biological Activity

A specific embodiment of the present invention provides a method for the screening of a derivative, fragment, analog or homolog of the Groucho protein for biological activity. The method includes contacting a derivative, fragment, analog or homolog of the Groucho protein with GIP and detecting the formation of a complex between the derivative, fragment, analog or homolog of the Groucho protein and GIP. Detection of the formation of the complex indicates that the Groucho protein derivative, fragment, analog or homolog, possesses biological (e.g., binding) activity. Similarly, an additional embodiment discloses a method for the screening a derivative, fragment, analog or homolog of GIP for biological activity. In this method, the derivative, fragment, analog or homolog of the protein is contacted with the Groucho protein. And formation of a complex between the derivative, fragment, analog or homolog of GIP and the Groucho protein is detected. Detecting the formation of the complex indicates that the GIP derivative, fragment, analog, or homolog possesses biological activity.

Modulation of Groucho Polypeptide Activity

The present invention discloses methods for screening Groucho protein●GIP complexes (and derivatives, fragments, analogs and homologs, thereof) for its ability to alter cell differentiation, cell proliferation, cell transformation and/or tumorigenesis in vitro and in vivo.

The Groucho protein●GIP complex (and derivatives, fragments, analogs and homologs, thereof) may also be screened for activity in modulating the fate of cell differentiation in vitro. The proteins and protein complex of the present invention may be screened by contacting cells with the protein or protein complex of the present invention and examining the cells for acquisition or loss of characteristics associated with a particular differentiated phenotype (a set of in vitro characteristics associated with a cell type).

The Groucho protein●GIP complex (and derivatives, fragments, analogs and homologs, thereof) may also be screened for activity to modulate the fate of cell differentiation in vivo in non-human test animal. In a specific embodiment of the present invention, the proteins and protein complex may be administered to a non-human test animal and the non-human test animals is subsequently examined for an increased incidence of cell differentiation in comparison with controls animals which were not administered the proteins or protein complex of the present invention. Accordingly, once a type of diseased cell has been shown to be amenable to treatment by modulation of Groucho protein●GIP complex activity, that disease or disorder may be treated or prevented by administration of a Therapeutic which modulates Groucho protein-GIP complex formation.

Groucho-GIP Interaction Assays

The present invention discloses methods for assaying and screening derivatives, fragments, analogs and homologs of GIP for binding to Groucho protein. The derivatives, fragments, analogs and homologs of the GIP which interact with Groucho protein may be identified by means of a yeast two hybrid assay system (see e.g., Fields & Song, 1989. Nature 340:245-246) or, a modification and improvement thereof.

The identification of interacting proteins by the improved yeast two hybrid system is based upon the detection of the expression of a reporter gene (hereinafter “Reporter Gene”), the transcription of which is dependent upon the reconstitution of a transcriptional regulator by the interaction of two proteins, each fused to one half of the transcriptional regulator. The bait Groucho protein (or derivative, fragment, analog or homolog) and prey protein (proteins to be tested for ability to interact with the bait protein) are expressed as fusion proteins to a DNA-binding domain, and to a transcriptional regulatory domain, respectively, or vice versa. In a specific embodiment of the present invention, the prey population may be one or more nucleic acids encoding mutants of GIP (e.g., as generated by site-directed mutagenesis or another method of producing mutations in a nucleotide sequence). Preferably, the prey populations are proteins encoded by DNA (e.g., cDNA, genomic DNA or synthetically generated DNA). For example, the populations may be expressed from chimeric genes comprising cDNA sequences derived from a non-characterized sample of a population of cDNA from mammalian RNA. In another specific embodiment, recombinant biological libraries expressing random peptides may be used as the source of prey nucleic acids.

The present invention discloses methods for the screening for inhibitors of GIP. In brief, the protein-protein interaction assay may be performed as previously described herein, with the exception that it is performed in the presence of one or more candidate molecules. A resulting increase or decrease in Reporter Gene activity, in relation to that which was present when the one or more candidate molecules are absent, indicates that the candidate molecule exerts an effect on the interacting pair. In a preferred embodiment, inhibition of the protein interaction is necessary for the yeast cells to survive, for example, where a non-attenuated protein interaction causes the activation of the URA3 gene, causing yeast to die in medium containing the chemical 5-fluoroorotic acid. See e.g., Rothstein, 1983. Meth. Enzymol. 101:167-180.

In general, the proteins comprising the bait and prey populations are provided as fusion (chimeric) proteins, preferably by recombinant expression of a chimeric coding sequence containing each protein contiguous to a pre-selected sequence. For one population, the pre-selected sequence is a DNA-binding domain that may be any DNA-binding domain, so long as it specifically recognizes a DNA sequence within a promoter (e.g., a transcriptional activator or inhibitor). For the other population, the pre-selected sequence is an activator or inhibitor domain of a transcriptional activator or inhibitor, respectively. The regulatory domain alone (not as a fusion to a protein sequence) and the DNA-binding domain alone (not as a fusion to a protein sequence) preferably, do not detectably interact, so as to avoid false-positives in the assay. The assay system further includes a reporter gene operably linked to a promoter that contains a binding site for the DNA-binding domain of the transcriptional activator (or inhibitor). Accordingly, binding of the Groucho protein fusion protein to a prey fusion protein leads to reconstitution of a transcriptional activator (or inhibitor), which concomitantly activates (or inhibits) expression of the Reporter Gene.

In a specific embodiment, the present invention discloses a method for detecting one or more protein-protein interactions comprising the following steps: (i) recombinantly-expressing the Groucho protein (or a derivative, fragment, analog or homolog thereof) in a first population of yeast cells of a first mating type and possessing a first fusion protein containing the Groucho protein sequence and a DNA-binding domain; wherein the first population of yeast cells contains a first nucleotide sequence operably-linked to a promoter which is “driven” by one or more DNA-binding sites recognized by the DNA-binding domain such that an interaction of the first fusion protein with a second fusion protein (comprising a transcriptional activation domain) results in increased transcription of the first nucleotide sequence; (ii) negatively selecting to eliminate those yeast cells in the first population in which the increased transcription of the first nucleotide sequence occurs in the absence of the second fusion protein; (iii) recombinantly expressing in a second population of yeast cells of a second mating type different from the first mating type, a plurality of the second fusion proteins; wherein the second fusion protein is comprised of a sequence of a derivative, fragment, analog or homolog of a GIP and an activation domain of a transcriptional activator, in which the activation domain is the same in each the second fusion protein; (iv) mating the first population of yeast cells with the second population of yeast cells to form a third population of diploid yeast cells, wherein the third population of diploid yeast cells contains a second nucleotide sequence operably linked to a promoter “driven” by a DNA-binding site recognized by the DNA-binding domain such that an interaction of a first fusion protein with a second fusion protein results in increased transcription of the second nucleotide sequence, in which the first and second nucleotide sequences can be the same or different and (v) detecting the increased transcription of the first and/or second nucleotide sequence, thereby detecting an interaction between a first fusion protein and a second fusion protein.

In a preferred embodiment, the bait (a Groucho protein sequence) and the prey (a library of chimeric genes) are combined by mating the two yeast strains on solid media for a period of approximately 6-8 hours. In a less preferred embodiment, the mating is performed in liquid media. The resulting diploids contain both types of chimeric genes (i.e., the DNA-binding domain fusion and the activation domain fusion). After an interactive population is obtained, the DNA sequences encoding the pairs of interactive proteins are isolated by a method wherein either the DNA-binding domain hybrids or the activation domain hybrids are amplified, in separate reactions. Preferably, the amplification is carried out by polymerase chain reaction (PCR; see e.g., Innis, et al., 1990. PCR Protocols (Academic Press, Inc., San Diego, Calif.)) utilizing pairs of oligonucleotide primers specific for either the DNA-binding domain hybrids or the activation domain hybrids. The PCR amplification reaction may also be performed on pooled cells expressing interacting protein pairs, preferably pooled arrays of interactants. Other amplification methods known within the art may also be used including, e.g., ligase chain reaction; Qp-replicase or the like. See e.g., Kricka, et al., 1995. Molecular Probing, Blotting, and Sequencing (Academic Press, New York, N.Y.).

In an additional embodiment of the present invention, the plasmids encoding the DNA-binding domain hybrid and the activation domain hybrid proteins may also be isolated and cloned by any of the methods well-known within the art. For example, but not by way of limitation, if a shuttle (yeast to E. coli) vector is used to express the fusion proteins, the genes may be subsequently recovered by transforming the yeast DNA into E. coli and recovering the plasmids from the bacteria. See e.g., Hoffman ,et al., 1987. Gene 57:267-272.

Pharmaceutical Compositions

The invention present discloses methods of treatment and prophylaxis by the administration to a subject of an pharmaceutically-effective amount of a Therapeutic of the invention. In a preferred embodiment, the Therapeutic is substantially purified and the subject is a mammal, and most preferably, human.

Formulations and methods of administration that can be employed when the Therapeutic comprises a nucleic acid as described above. Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (see, e.g., Wu & Wu, 1987. J. Biol. Chem. 262:4429-4432); (iv) construction of a Therapeutic nucleic acid as part of a retroviral or other vector, and the like.

Methods of administration include, e.g.,, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

In another embodiment of the present invention, the Therapeutic may be delivered in a vesicle, in particular a liposome. See e.g., Langer, 1990. Science 249:1527-1533. In yet another embodiment, the Therapeutic can be delivered in a controlled release system including, e.g.: a delivery pump (see e.g., Saudek, et al., 1989. New Engl. J. Med 321:574 and a semi-permeable polymeric material (see e.g., Howard, et al., 1989. J. Neurosurg. 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (see e.g., Joliot, et al., 1991. Proc. Natl. Acad. Sci. USA 88:1864-1868), and the like. Alternatively, a nucleic acid Therapeutic can be introduced intracellularly and incorporated within host cell DNA for expression, by homologous recombination.

The present invention also provides pharmaceutical compositions. Such compositions comprise a therapeutically-effective amount of a Therapeutic, and a pharmaceutically acceptable carrier. As utilized herein, the term “pharmaceutically acceptable” means approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. However, suitable dosage ranges for intravenous administration of the Therapeutics of the present invention are generally about 20-500 micrograms (ag) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The present invention also provides a pharmaceutical pack or kit, comprising one or more containers filled with one or more of the ingredients of the pharmaceutical compositions and Therapeutics of the present invention. Optionally associated with such container(s) may be a notice in the form prescribed by a governmental agency regulating the manufacture, use or sale of pharmaceuticals or biological products, which notice reflects approval by the agency of manufacture, use or sale for human administration.

The following EXAMPLES are presented in order to more fully illustrate the preferred embodiments of the invention. These EXAMPLES should in no way be construed as limiting the scope of the invention, as defined by the appended claims.

### EXAMPLES

### Example I

Experimental Procedures

a) DNA Constructs

cDNAs encoding full-length proteins were prepared according to the relevant reference: Nkx2.2 (Briscoe et al, 2000), Nkx2.9 (Pabst et al., 1998), Nkx6.1 (Jensen et al., 1996), Nkx6.2 (Komuro et al., 1993), Dbx2 (Rangini et al., 1991), Pax6 (Kozmik et al., 1997), Grg5 (Mallo et al., 1993). The following deletion constructs were generated: NKx2.2^{ΔTN} (Δaa 2-26), NKx2.9^{ΔTN} (Δaa 2-27), Nkx6.1^{ΔTN} (Δaa 90-113), Nkx6.2^{ΔTN} (Δaa 2-73), Dbx2^{Δeh1} (Δaa 2-48), and Grg5^{ΔQ} (Δaa 2-129). The following fusion constructs were generated: DNA encoding the homeodomains of Nkx2.2 (aa 120-189), Nkx6.1 (aa231-305) and Dbx2 proteins (96-156), and the paired domain of Pax6 (aa 1-131) was fused either to i) DNA encoding a myc tagged Engrailed repressor protein (aa 2-298, Genbank accession number M10017), ii) a VP16 trans-activation domain (aa 400-488, Genbank accession number NP_{—}044650) or iii) a myc-tag only. Full-length, deletion and hybrid constructs were subsequently cloned into pCMXGAL4, pCAGGS, and RCASBP(B) vectors.

b) In Vitro Binding Assays

Glutathione S-transferase (GST) co-precipitation experiments were performed essentially as described (Ren et al. 1999), except that S³⁵-labelled proteins were incubated with GST-Gro for lhr at +4° C. GST and GST-Gro constructs were provided by R. Goldstein, and GST and GST-Gro fusion protein were produced and isolated as described (Ren et al., 1999). ³⁵S-labelled Homeodomain and Gro/TLE proteins (rNkx6.1, rNkx6.1^{ΔTN}, mNkx6.2, cNkx2.2, cNkx2.2^{ΔTN}, mNkx2.9, mPax6, mPax7, mDbx1, mDbx2, mGrg5 and mGrg5^{ΔQ}) were synthesized using the TNT coupled rabbit reticulocyte lysate (Promega). Immuno-precipitation (IP) assays using rabbit Nkx2.2 (Ericson et al., 1997) and Nkx6.1 antibodies (Jensen et al., 1996) were performed as described by Current protocols 2^{nd} ed. ³⁵S-labelled mGrg4 was synthesized using the TNT coupled rabbit reticulocyte lysate. Electric mobility shift assays were performed as described (Jorgensen et al., 1999). Briefly, the isolated homeodomains of Nkx6.1, Nkx6.2, Nkx2.2 and Nkx2.9HD were synthesized using the TNT coupled rabbit reticulocyte lysate and tested for their binding specificity in vitro using ³²P-labelled double stranded DNA oligonucleotides containing defined Nkx6.1 (Jorgensen et al., 1999) or Nkx2.1 binding sites (Damante et al., 1996).

c) COS-7 Cell Transfection Assays

Cos-7 cells were transfected as described (Beatus et al., 1998). Each transfection contained a fixed amount (4.7 μg) of plasmid DNA. Plasmids: 100 ng MH100-tk-luciferase reporter plasmid (Kang et al. 1993; Perlmann and Jansson, 1995), 50 ng CMV-lacZ plasmid, 200 ng gal4-expression plasmid (gal-only, gal-Nkx2.2, gal-Nkx2.2 ^{ΔTN}, gal-Nkx2.9, gal-Nkx2.9^{ΔTN}, gal-Nkx6.1, gal-Nkx6.1^{ΔTN}, gal-Nkx6.2, gal-Nkx6.2^{ΔTN}). Various amounts (300-1200 ng) of mGrg4, mGrg5, mGrg5^{ΔQ} and/or pcDNA3 (mock) were included as indicated. Cells were harvested after 42 hours and luciferase activity was assayed as described (Beatus et al., 1998). The relative luciferase activity in individual transfections was calculated and compared with the value of the gal-only control set as 1. Data points represent the average of at least three independent transfections ±SD.

d) In Ovo Electoporation

cDNA encoding full-length, truncated or hybrid variants of homeodomain proteins of Nkx2.2, Nkx6.1, Dbx2 and Pax6 (See DNA constructs above; Briscoe et al., 2000; Pierani et al., 2001) were cloned into RCASBP(B) or pCAGGS and Grg5 and Grg5^{ΔQ} were inserted into pCAGGS. Expression vectors were electroporated in ovo into the neural tube of HH stage 10-12 embryos. Embryos were allowed to develop for 24-48 hours and then fixed and processed for immunohistochemistry and in situ hybridization analyses (Briscoe et al., 2000). In co-electroporation experiments with Nkx6.1 and Grg5/Grg5^{ΔQ}, vectors were mixed in a ratio of 2:1.

e) Immunohistochemistry and In Situ Hybridization

Mouse (White et al., 1992) and rabbit VP16 antibodies (Abs) were gifts from L Tora and S. Arber, respectively. 9E10 c-myc Ab's was obtained from the Developmental Studies Hybridoma Bank. Other Ab reagents and protocols have been described (Yamada et al., 1993; Ericson et al., 1997; Tanabe et al., 1998; Briscoe et al., 1999; Pierani et al., 1999). In situ hybridization was performed essentially as described by (Schaeren-Wiemers and Gerfin-Moser, 1993), using probes for Siml, Nkx2.2, Grg5, Nkx6.1, Dbx2, Ptc and Shh (Briscoe et al., 2000; Pierani et al., 1999; Roelink et al., 1994;.Marigo and Tabin, 1996).

### Example II

Nkx Proteins Interact with Groucho/TLE Corepressors

The TN domain, or Nkx decapeptide (Lints et al., 1993; Harvey, 1996), a conserved ca. 11 amino acid long motif present in Nkx2.2, Nkx6.1 and Nkx6.2, shows high sequence similarity to the core region of the engrailed homology-1 (eh1) domain (FIG. 1A; Logan et al., 1992; Smith and Jaynes, 1996; Jimènes et al., 1997). The ehI domain is a protein motif found in the amino terminal region of the homeodomain protein Engrailed (En), a known transcriptional repressor (Jaynes and O'Farrell, 1991; John et al., 1995). The ehI domain is known to interact with members of the Gro/TLE family of corepressors (Tolkunova et al., 1998; Jimenez et al., 1999). Sequence analysis revealed that Nkx2.9 possesses an amino-terminal eh1-like motif, albeit one that is less conserved than in the three other Nkx family members, as shown in FIG. 1A. These findings raise the possibility that Nkx proteins influence neural pattern by acting as Gro/TLE-dependent transcriptional repressors.

FIG. 1A shows the TN-domain (white) positioned amino-terminal to the homeodomain (dark gray) in Nkx proteins. The homology to the core sequence of the eh1-domain present in the Engrailed (En) repressor protein (Logan et al., 1992; Smith and Jaynes, 1996) is also shown. Listed sequences include the TN-domains of mouse Nkx2.2 (SEQ ID NO: 1) and Nkx2.9 (SEQ ID NO: 2), rat Nkx6.1 (SEQ ID NO: 3), chick Nkx6.2 (SEQ ID NO: 4), Drosophila Vnd (SEQ ID NO: 5) and the eh1 domain in En (SEQ ID NO: 6). An Nkx TN-domain consensus (SEQ ID NO: 7) sequence was obtained by comparing the deduced sequence of 15 members of the Nkx protein family. Highly conserved amino acid positions are indicated with filled circles (>90%) or diamonds (70-90%).

To determine whether Nkx proteins implicated in ventral neural patterning interact physically with Gro/TLE corepressors, it was first determined if Drosophila Groucho (Gro), the prototypic and best defined Gro/TLE class protein (Hartley et al, 1988; Paroush et al., 1994), could interact with Nkx6.1, Nkx6.2, Nkx2.2 and Nkx2.9 in a glutathione S-transferase (GST) co-precipitation assay. GST-Gro was found to bind selectively to Nkx6.1, Nkx6.2, Nkx2.2 and Nkx2.9. FIG. 1B depicts the interaction between Drosophila Groucho (Gro) and Nkx proteins in vitro. A GST-Gro fusion protein immobilised on glutathione-sepharose beads interacted with ³⁵S-labeled Nkx2.2, Nkx2.9, Nkx6.1 and Nkx6.2. GST protein alone did not bind any of these Nkx proteins. Input represents 10% of the amount ³⁵S-labelled Nkx protein used in the binding assays.

An immuno-precipitation assay was then used to test if mammalian Gro/TLE family members could also bind to Nkx proteins, and if the TN domain is required for this interaction. In these experiments, two Nkx proteins (Nkx2.2 and Nkx6.1) that exhibit distinct DNA binding specificities ( See FIG ID; Jorgensen et al., 1999; Watada et al., 2000), and neural patterning activities (Briscoe et al., 1999; 2000) were used. Carboxy-terminal directed antibodies that recognise both full-length proteins as well as truncated variants lacking the TN domain were used (Ericson et al., 1997; Jensen et al., 1996). The interaction of Nkx2.2 and Nkx6.1 with Grg4 was investigated because the Gr4 gene is expressed at high levels in the ventral neural tube.

The immuno-precipitation analysis shown in FIG. 1C indicates that ³⁵S-labeled mouse Grg4 can interact with Nkx2.2 and Nkx6.1 protein immobilised on antibody-proteinA-sepharose complexes. Deletion of the TN domain in Nkx2.2 and Nkx6.1 reduced the interaction with Grg4 (9-fold for Nkx2.2ATN and 5-fold for Nkx6.1ΔTN, compared to the full-length proteins). Thus, a TN domain-deleted version of Nkx6.1 interacts with Grg4, albeit much more weakly than the corresponding full-length protein. Therefore, regions of Nkx6.1 other than the TN domain appear to contribute to the interaction of this protein with Grg4 (Choi et al., 1999a; Choi et al., 1999b). These data provide evidence that Nkx proteins expressed in the ventral tube can interact with Gro/TLE corepressors in vitro, and that the TN domain mediates, in large part, this interaction. No significant interaction was detected between Grg4 and antibody-proteinA-sepharose complexes. Input represent 10% of the amount ³⁵S-labelled Grg4 protein used in the binding assays.

The data presented in FIG ID indicate that the homeodomains (HDs) of Nkx6.1 and Nkx6.2, but not that of Nkx2.2 or Nkx2.9, bind to a defined Nkx6.1 binding site (Jorgensen et al., 1999) in electric mobility shift assays (EMSA) and that Nkx2.2HD and Nkx2.9HD, but not the Nkx6.1HD or Nkx6.2HD, interact with a defined Nkx2.1 binding site (Watada et al, 2000) in EMSA. The deletion proteins did not differ from the corresponding wild-type proteins in binding to their respective target DNA sequences (not shown).

### Example III

Nkx Proteins Act as Transcriptional Repressors

To determine whether Nkx6.1, Nkx6.2, Nkx2.2 and Nkx2.9 activate or repress transcription when recruited to an active promoter, their activity in in vitro transfection assays was assessed (Perlmann and Jansson, 1995). Full-length Nkx6.1, Nkx6.2, Nkx2.2 and Nkx2.9, or truncated versions of these proteins lacking the TN domain, were fused to the DNA binding domain of the yeast transcription factor Gal4. These Gal-Nkx-fusion constructs were co-transfected into COS-7 cells, together with a reporter plasmid containing four tandem UAS sequences positioned upstream of a minimal thymidine kinase (TK) promoter and the luciferase reporter gene (Kang et al. 1993 ). Results are depicted in FIG 1E.

In this assay, introduction of Gal-Nkx6.1, Gal-Nkx6.2 and Gal-Nkx2.2 into COS-7 cells reduced the level of transcription approximately 15-fold, and Gal-Nkx2.9 reduced the level of transcription 5-fold, compared to the control (gal-only) when cotransfected with a constitutively active UAS-luciferase reporter plasmid into COS7 cells (Perlmann and Jansson, 1995). Deletion of the TN domain in Nkx2.2 (Gal-Nkx2.2ΔTN) and Nkx2.9 (Gal-Nkx2.9ΔTN) greatly reduced the repressive activity of these transcription factors, and did not unmask any transcriptional activation activity. Removal of the TN domain in Nkx6.1 (Gal-Nkx6.1ΔTN) and Nkx6.2 (Gal-Nkx6.2ΔN) resulted in a 8-10 fold reduction in repressor activity compared to full-length proteins. However, both Nkx6.1 and Nkx6.2 variants lacking the TN domain exhibited weak repressor activity.

To determine if the TN domain-dependent repressor activity of Nkx proteins is sensitive to the presence of Gro/TLE proteins, Gal-Nkx constructs were co-transfected with varying levels of an expression vector encoding mouse Grg4. Increasing the level of Grg4 resulted in a progressive, up to 10 fold, increase in the repressor activity of all full-length Nkx proteins. Increasing amounts of Grg4 and had no corepressor activity in experiments with gal-Nkx2.2ΔTN or gal-Nkx2.9ΔTN but weakly enhanced the repressor activity of gal-Nkx6.1ΔTN or gal-Nkx6.2ΔTN (˜2-fold). Error bars indicate standard deviation (SD) of 3-6 independent transfections. These data provide evidence that the residual repressor activity of Nkx6.1 and Nkx6.2 variants lacking the TN domain is mediated through an interaction with gro/TLE corepressors. Together, these data show that Nkx6.1, Nkx6.2, Nkx2.2 and Nkx2.9 act as transcriptional repressors in vitro, and suggest that their activities rely on the ability of the TN domain to recruit Gro/TLE proteins.

### Example IV

Nkx Proteins Function as TN Domain-Dependent Repressors

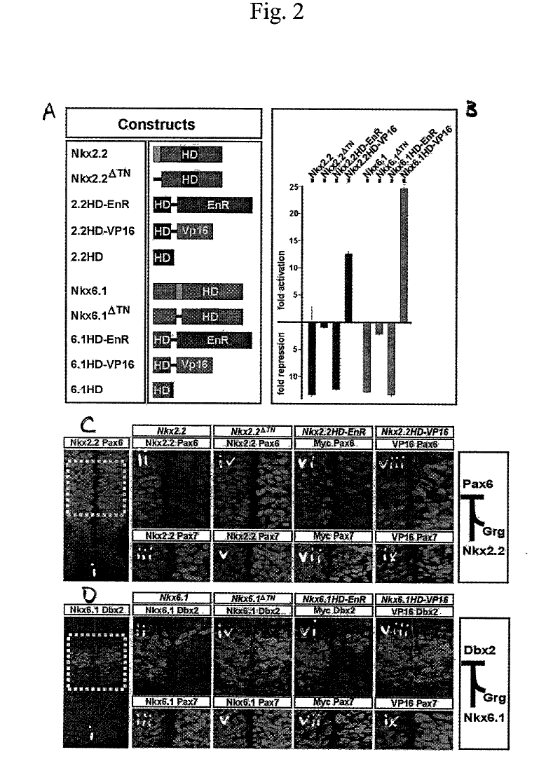

Several Nkx proteins, including Nkx2.1 and Nkx2.2, have been shown to function as transcriptional activators (Choi et al., 1999; Watada et al., 2000; Civitareale et al., 1989; Bohinski et al., 1994), and the COOH-terminal domain of Nkx2.2 contains a potent transactivation domain (Watada et al., 2000). These observations raised the possibility that Nkx proteins might also be capable of regulating neural pattern through their function as activators. To address this possibility, hybrid activator proteins consisting of the Nkx2.2 or Nkx6.1 homeodomains coupled to the transactivation domain of the viral protein VP16 were analyzed for influence on class I homeodomain protein expression in vivo. Neither the Nkx2.2HD-VP16 or Nkx6.1HD-VP16 hybrid activator proteins had any detectable influence on class I protein expression in vivo (FIG. 2C), despite their strong transcriptional activation activity in cell line reporter assays (FIG. 2B). These results support the idea that Nkx proteins regulate neural pattern through their actions as transcriptional repressors.

To determine whether Nkx proteins function as TN domain-dependent repressors during the patterning of neural progenitors in vivo, chick in ovo electroporation (Muramatsu et al., 1997) was used to express full-length or TN domain-deleted variants of Nkx proteins and their ability to repress class I progenitor homeodomain protein expression was analyzed. In these studies, Nkx2.2 and Nkx6.1 proteins were used because their neural patterning activities have been characterized in detail, and because their complementary class I proteins (Pax6 and Dbx2, respectively) have been identified (Ericson et al., 1997; Briscoe et al, 1999; Briscoe et al., 2000; Sander et al., 2000). To determine whether the neural patterning activity of Nkx proteins is mediated exclusively by their ability to function as repressors, hybrid repressor proteins consisting of the homeodomains of Nkx2.2 (Nkx2.2HD) or Nkx6.1 (Nkx6.1 HD) coupled to the repressor domain of En (EnR) were generated.

FIG. 2A depicts a schematic representation of the constructs expressed in the chick neural tube by in ovo electroporation. The constructs outlined are: full-length Nkx2.2 and Nkx6.1; Nkx2.2ΔTN and Nkx6.1ΔTN with the TN domain selectively deleted; hybrid proteins consisting of the Nkx2.2 homeodomain (2.2HD) or Nkx6.1 homeodomain (6.1 HD) coupled to a myc-tagged En repressor domain (EnR) or to the transactivation domain of the viral protein VP16 (VP16).

The constructs outlined in panel 2A were fused to Gal4 and examined for their activity in a transcription reporter assay in COS7 cells (Perlmann and Jansson, 1995). Data shown in FIG. 2B indicate that Gal-Nkx2.2 and Gal-Nkx6.1 reduced the transcriptional activity of the luciferase reporter gene 12,9 and 13,5 fold, respectively, compared to the Gal-only control. Ablation of the TN domain in Nkx2.2 and Nkx6.1 (Nkx2.2ΔTN and Nkx6.1ΔTN) essentially abolished the repressor activity of these proteins. Gal-Nkx2.2-EnR and Gal-Nkx6.1-EnR reduced the transcriptional activity 12,4- and 13,4-fold respectively. Gal-Nkx2.2-VP16 and Gal-Nkx2.2-VP16 enhanced transcription 12,7- and 24,9-fold respectively. Error bars indicate standard deviation (SD) of 3 independent transfections.

FIG. 2C indicates that Nkx2.2 and Pax6 are expressed in mutually exclusive domains in the spinal cord of HH stage 20 chick embryos. Misexpression of full-length Nkx2.2 at ectopic dorsal positions in the neural tube results in a cell-autonomous repression of Pax6 expression (See also Briscoe et al., 2000). In contrast, forced dorsal expression of Nkx2.2ΔTN failed to change the level of Pax6 expression. Misexpression of full length Nkx6.1 in more dorsal regions of the neural tube results in a cell-autonomous in repression of the class I protein Dbx2 (see also Briscoe et al., 2000). In contrast, after ectopic expression of Nkx6.1ΔTN, over 85% of Nkx6.1ΔTN⁺ cells located within the intermediate neural tube maintained Dbx2 expression. However, 15% of Nkx6.1ΔTN⁺ cells within the normal domain of Dbx2 expression did lack Dbx2 expression (data not shown), implying that deletion of the TN domain of Nkx6.1 reduces, but does not completely abolish, its ability to repress Dbx2 in vivo. Together, these findings identify the TN domain as a critical motif for effective Nkx protein-mediated repression of class I progenitor homeodomain proteins in vivo. Box (i), indicates the analyzed domain. Forced expression of Nkx2.2 in the dorsal parts of the neural tube repressed Pax6 expression (box ii) but not Pax7 expression (box iii). Expression of Nkx2.2^{ΔTN} did not repress Pax6 (box iv) or Pax7 (box v) expression. Expression of Nkx2.2HD-EnR repressed Pax6 (box vi) but not Pax7 (box vii) expression. Forced expression of Nkx2.2-VP16 did not affect the expression of Pax6 (box viii) or Pax7 (box ix).

FIG. 2D indicates that Nkx6.1 and Dbx2 are expressed in mutually exclusive domains in the ventral spinal cord of HH stage 20 chick embryos (i) Bracket box indicates analyzed domain in (ii, iv, vi, and viii). Misexpression of Nkx6.1 repressed Dbx2 (>70%/section, n=5) (ii) but not Pax7 expression (iii). Most cells in the intermediate neural tube that express Nkx6.1_{ΔTN} also express Dbx2 (iv) and expression of Pax7 was unaffected (v). Forced expression of Nkx6.1^{ΔTN}, however, resulted in a small reduction in numbers of Dbx2⁺ cells (12%±6% /section n=5). Forced expression of Nkx6.1HD-EnR repressed expression Dbx2 (VI) (>70% cells/section, n=5 sections) but not Pax7 (vii). Expression of Nkx6.1HD-VP16 had no effect on Dbx2 (VIII) or Pax7 (IX) expression. The repressive efficiency was determined by comparing the numbers of Dbx2⁺ cells on the electroporated side compared to contralateral control.

Ectopic expression of Nkx2.2HD-EnR and Nkx6.1HD-TN mimicked the activity of the corresponding full-length Nkx proteins and repressed Pax6 and Dbx2, respectively (FIGS. 2C and 2D). The expression of the class I proteins Pax7 was unaffected by expression of either the full-length, the TN domain deleted variants, or the hybrid repressor-forms of Nkx2.2 and Nkx6.1 (FIG. 2C; Briscoe et al., 2000). In addition, ectopic expression of the Nkx2.2 and Nkx6.1 homeodomains alone, in the absence of the EnR domain, did not change the pattern of Pax6 and Dbx2 expression in vivo (data not shown). Together, these observations support the idea that the repressor activity of these Nkx proteins is sufficient to mediate their neural patterning activities.

### Example V

Nkx-Mediated Transcriptional Repression Controls Ventral Neuronal Fate

Nkx proteins play a central role in the specification of neuronal fate in the ventral neural tube (Briscoe et al., 1999; Briscoe et al., 2000; Sander et al., 2000; Sussel et al., 1999). It was determined whether the repressor functions of Nkx proteins are also sufficient to respecify ventral neuronal fates. To test this, the full length, TN domain-deleted, and EnR and VP16 hybrid variants of Nkx2.2 and Nkx6.1 (FIG. 2A) were expressed ectopically in the neural tube, and changes in neuronal fate were analyzed.

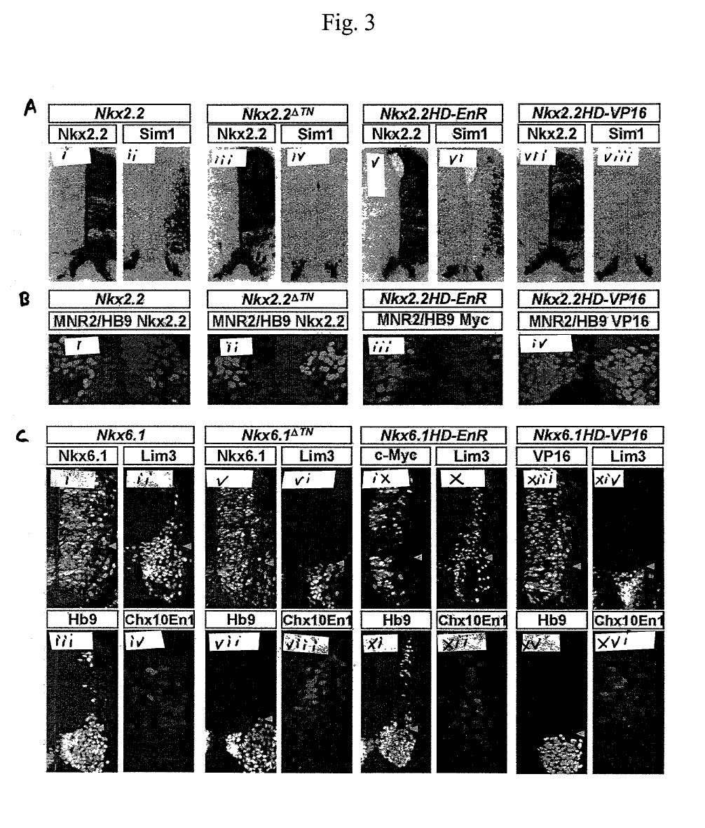

The data in FIG. 3 indicate that ectopic expression of Nkx2.2HD-EnR and Nkx6.1 HD-EnR mimicked the ability of the corresponding full-length Nkx proteins to respecify neuronal fate. Misexpression of the Nkx2.2HD-EnR protein induced ectopic dorsal V3 neurons, as indicated by the expression of Sim1 along the entire dorsoventral axis of the neural tube, with an efficiency similar to that of full-length Nkx2.2. In contrast, expression of either the Nkx2.2ΔTN protein or the Nkx2.2HD-VP16 protein had no Sim1-inductive activity. However, expression of Nkx2.2HD-EnR resulted in the ectopic expression of endogenous Nkx2.2 in a few scattered cells within the ventral neural tube (data not shown). The activation of endogenous Nkx2.2 expression is likely to reflect the early repression of Pax6 expression (Ericson et al., 1997). However, the presence of scattered ectopic Nkx2.2⁺ cells does not account for the broad activation of Sim1 expression observed in these experiments. Thus, induction of ectopic V3 neurons is likely to reflect in large part, the activity of the Nkx2.2HD-EnR protein.

Whether the ability of Nkx2.2 to inhibit motor neuron generation (Ericson et al., 1997; Broscoe et al, 1999, Briscoe et al., 2000) resides in its repressor function was also examined. Ectopic expression of Nkx2.2HD-EnR within the pMN progenitor domain mimicked the ability of full-length Nkx2.2 to repress expression of the motor neuron progenitor determinants MNR2 and Lim3 (Tanabe et al., 1998; Sharma et al., 1998), and the post-mitotic motor neuron markers HB9 and Isl1 (Arber et al., 1999; Thaler et al., 1999; Pfaff et al., 1996). In contrast, neither the Nkx2.2ΔTN nor the 2.2HD-VP16 proteins affected the generation of motor neurons. Thus, the repressor function of Nkx2.2 appears sufficient to account for its neuronal patterning activity in the spinal cord.

FIG. 3A shows that forced expression of Nkx2.2 induced ectopic V3 neurons, as indicated by SimI expression (box i, ii). Expression of Nkx2.2ATN did not induce ectopic Siml expression (box iii, iv). Expression of Nkx2.2HD-EnR induced ectopic Siml cells at a similar efficiency as Nkx2.2 (box v, vi). Expression of Nkx2.2HD-VP16 did not induce ectopic Sim1 expression (box vii, viii).

FIG. 3B shows forced expression of Nkx2.2 and Nkx2.2HD-EnR in motor neuron progenitors suppresses expression of the motor neuron markers MNR2 and HB9 (box i, iii).

Expression of Nkx_{2.2}^{ΔTN} or Nkx2.2HD-VP16 did not alter the expression of MNR2 and HB9 in differentiating motor neurons generated (box ii, iv).

FIG. 3C indicates that misexpression of Nkx6.1 induced ectopic motor neurons, as shown by the ectopic dorsal expression of Lim3 (44 cells/section ±10 cells) and HB9 (33 cells/section ±10 cells) (boxes i, ii and iii). Expression of Nkx6.1^{ΔTN} did not induce ectopic Lim3 or Hb9 expression (boxes v, vi and vii). vNkx6.1HD-EnR induced ectopic dorsal expression of Lim3 (47 cells/section ±12 cells) and HB9 (35 cells/section) (boxes ix, x and xi). Forced expression ofNkx6.1HD-VP16 did not induce ectopic expression of Lim3 or HB9 (boxes xiii, xiv and xv).

Misexpression of Nkx6.1 (box iv) and Nkx6.1HD-EnR (box xii) induced ectopic V2 neurons in the ventral p1 domain, as indicated by a dorsal expansion of Chx10 expressing cells in the ventral neural tube. Induction of ectopic V2 neurons in these experiments was accompanied by reduced the number of En1 expressing VI neurons. No significant alteration in the ratio of V2 or V1 neurons could be detected in experiments with Nkx6.1^{ΔTN} (box viii) or Nkx6.1HD-VP16 (box xvi).

The contribution of repression to the patterning activity of Nkx6.1 was also evaluated. Expression of the Nkx6.1HD-EnR hybrid repressor protein mimicked the activity of full-length Nkx6.1 protein to induce the motor neuron markers, MNR2, Lim3, HB9 and Isl1 at ectopic positions in the dorsal neural tube. In addition, when misexpressed within the p1 and p2 progenitor domains, Nkx 6.1HD-EnR mimicked the ability of full-length Nkx6.1 to induce V2 neurons, and to suppress the generation of V0 and V1 neurons. Neither Nkx6.1ΔTN or 6.1HD-VP16 induced motor neurons or V2 neurons in dorsal positions, nor were V1 neurons repressed. In addition, expression of Nkx6.1ΔTN or 6.1HD-VP16 within the pMN and p2 domains did not affect the generation of motor neurons or V2 neurons. Taken together, this analysis indicates that the respecification of neuronal fates by Nkx2.2 and Nkx6.1 can be attributed to their function as transcriptional repressors.

### Example VI

Certain Class I HD Proteins Interact with Groucho/TLE Proteins

The establishment of ventral progenitor domains relies on mutual repressive interactions between class I and class II proteins. The TN domain-dependent repressor activity of class II proteins raised the possibility that class I proteins also function as transcriptional repressors. Six class I homeodomain proteins, comprising members of the Pax, Dbx, and Irx families, have been implicated in ventral neural patterning (Goulding et al., 1993; Ericson et al., 1996; 1997; Pierani et al., 1999; Briscoe et al., 2000).

It was determined whether any of these class I proteins possessed eh1-like domains. The Dbx1 and Dbx2 proteins define the p0/p1 and p1/p2 progenitor domain boundaries, and both proteins contain an amino terminal ehI motif. Pax3 and Pax7 which define the dorsoventral boundary of the neural tube also possess eh1 motifs. However, eh1 motifs were not detected in Irx3 or Pax6, despite evidence that Pax6 represses Nkx2.2 expression in neural progenitor cells (Ericson et al., 1997, Briscoe et al., 2000), and that Irx3 represses motor neuron generation (Briscoe et al., 2000). Thus, only four of the six class I homeodomain proteins contain recognizable eh1 domains. FIG. 4A shows an alignment of the class I genes Dbx1 (SEQ ID NO: 8), Dbx2 (SEQ ID NO: 9), Pax3 (SEQ ID NO: 10) and Pax7 (SEQ ID NO: 11). The Nkx TN-domain consensus sequence (SEQ ID NO: 7) and the eh1 domain of En (SEQ ID NO: 6) are shown as comparison. No eh1-like motifs were detected in the class I proteins Pax6 or Irx3.

To determine if class I proteins interact physically with groucho/TLE coprepressors, four representative class I proteins, Dbx1, Dbx2, Pax7 and Pax6, were analyzed for binding to Drosophila Gro in vitro in co-precipitation assays. GST-Gro fusion protein immobilised on glutathione-sepharose beads interacted with ³⁵S-labeled Dbx1, Dbx2 and Pax7 but not with ³⁵S-labeled Pax6, consistent with the absence of a defined enI motif in this class I protein. Immobilised GST protein alone did not bind any of these class I proteins. Input represents 10% of the amount ³⁵S-labelled class 1 protein used in the binding assays.

To determine whether the presence or absence of eh1-like domains in class I proteins predicts their in vivo patterning activities as transcriptional repressors or activators, the activities of Dbx2 and Pax6 were assayed. The complementary class II proteins for Dbx2 and Pax6 (Nkx6.1 and Nkx2.2, respectively) have been identified (Ericson et al., 1997; Briscoe et al., 1999; Briscoe et al., 2000; Pierani et al.,1999).

To define if the patterning activities of Dbx2 involves eh1 domain-dependent transcriptional repression, variants of Dbx2 that lacked the eh I motif (Dbx2ΔTN), as well as hybrid Dbx2 homeodomain (Dbx2HD) constructs coupled to the EnR or VP1 6 domains were generated and expressed in the neural tube. As shown in FIG. 4B-4E, Dbx2HD-EnR mimicked full length Dbx2 in its ability to repress Nkx6.1 expression, but, Dbx2ΔTN and Dbx2HD-VP16 exhibited no apparent patterning activity. These data support the idea that the class I protein Dbx2 functions as a transcriptional repressor in vivo, and show this activity requires the eh1 domain.

Forced expression data for Dbx2 are shown in FIGS. 4F-4I. Forced expression of Dbx2 in the ventral third of the neural tube repressed Nkx6.1 expression (4F), forced expression of Dbx2^{ΔEh1} did not affect the expression of Nkx6.1 (4G), ventral expression of Dbx2HD-EnR repressed Nkx6.1 expression (4H) whereas expression of Dbx2HD-VP16 had no influence on Nkx6.1 expression (4I).

Pax6 lacks an obvious Gro/TLE binding domain and therefore hybrid repressor and activator forms of Pax6 were generated, to determine if the observed repression of Nkx2.2 is mediated by activator or repressor functions of Pax6. In these experiments, the DNA-binding Paired domain (Epstein et al., 1994; Kozmik et al., 1997) of Pax6 (Pax6PD) was fused to the EnR or VP16 and these hybrid Pax6 proteins were expressed in the ventral neutral tube. FIGS. 4J-4M show forced expression of Pax6. Forced expression of Pax6 in the ventral spinal cord (4J) repressed the expression of Nkx2.2 in the p3 domain (4K). Misexpression of Pax6PD-VP16 repressed Nkx2.2 expression within the p3 domain (4L). Forced expression of Pax6PD-EnR induced Nkx2.2 expression at ectopic dorsal positions of the ventral neural tube (4M). In these experiments, Pax6PD-VP16 mimicked the ability of the Pax6 full-length protein to suppress Nkx2.2 expression within the p3 domain. In contrast, expression of Pax6PD-EnR induced Nkx2.2 at ectopic positions, dorsal to the p3 domain. These findings suggest that Pax6, in contrast to most class I and class II proteins, acts as a transcriptional activator in neural patterning in vivo. They also suggest that the repression of Nkx2.2 by Pax6 is mediated indirectly through the activation of an intermediary repressor protein.

The inverse activity of the repressor form of Pax6, compared to the full-length Pax6 differs from the analysis of class I and class II repressor proteins, in which activator variants of these repressor proteins lacked any obvious in vivo activity (FIGS. 2B, 2C; FIG. 3; FIG. 4C).

### Example VII

Grg4 Expression in the Developing Neural Tube.

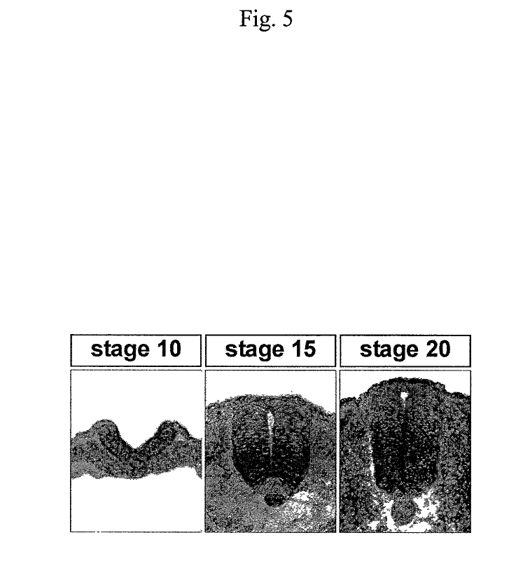

The role of the eh1-like domain in mediating the repressive activities of class I and class II progenitor homeodomain proteins is consistent with the idea that these proteins function in vivo by recruiting members of the Gro/TLE class of transcriptional corepressors. To assess the involvement of Gro/TLE proteins in dorsoventral patterning in vivo, the pattern of expression of Gro/TLE genes in the ventral neural tube of chick embryos was examined. Chick Grg4 was expressed at uniform levels at early neural plate and neural fold stages. But after neural tube closure, Grg4 was expressed in a graded manner, with a higher level of expression in ventral and intermediate regions of the neural tube. In in situ labeling experiments shown in FIG. 5, depict the expression of Grg4 in developing chick spinal cord. Grg4 is uniformly expressed in the neural plate of HH stage 10 chick embryos. At HH stages 15 and 20, Grg4is expressed in a graded fashion with higher levels detected in ventral and intermediate regions, compared to the dorsal third of the neural tube. The expression of Gro/TLE genes in the mouse was also analyzed. In e10 embryos, Grg4 and Grg3 were expressed at higher levels in the ventral and intermediate parts of the neural tube, and at lower levels more dorsally (See, Muhr et al., Cell, 104:861-873, 2001; also Miyasaka et al., 1993; Koop et al., 1996; Leon and Lobe, 1997). Grgl was not detected at significant levels in the neural tube at this stage, whereas expression of Grg5, a gene that encodes s small endogenous Gro/TLE-like protein with a dominant negative activity (Miyasaka et al., 1993; Roose et al., 1998; Ren et al., 1999), was restricted primarily to post-mitotic neurons. Thus, the pattern of expression of Grg genes in the chick and mouse neural tube is consistent with a role for these proteins as mediators of the repressor activities of class I and class II homeodomain proteins in vivo.

### Example VIII

Ectopic Expression of Grg5 Perturbs Neural Tube Patterning.

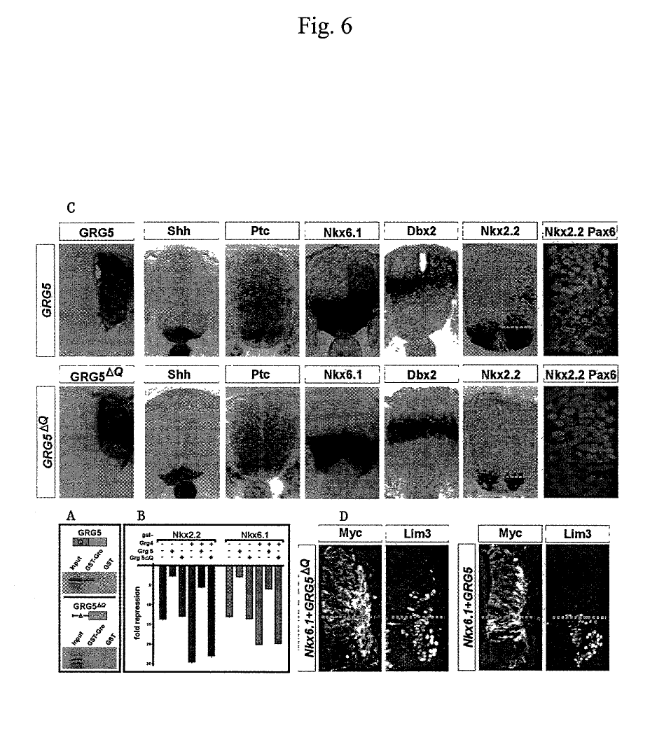

If Gro/TLE proteins mediate the effects of class I and class II homeodomain proteins, neural patterning might be perturbed under conditions in which Gro/TLE function has been blocked or attenuated. To block Gro/TLE protein function in vivo, the activity of Grg5, a gene encoding a variant Gro/TLE protein that possesses a dominant negative activity (Roose et al., 1998; Ren et al., 1999) was evaluated. The inhibitory activity of Grg5 depends on a amino terminal “Q” domain, which appears to bind and inhibit the activities of Gro/TLE class corepressors (Miyasaka et al., 1993; Roose et al., 1998; Ren et al., 1999). Consistent with this view, Grg5, but not a variant of Grg5 lacking the Q-domain (Grg5^{ΔQ}), interacted physically with Drosophila Gro in co-precipitation assays. Neither Grg5 nor Grg5^{ΔQ} bound immobilised GST protein (FIG. 6A).

In addition, expression of Grg5, but not Grg5^{ΔQ}, reduced the ability of Nkx2.2, Nkx6.1, Nkx2.9, and Nkx6.2 to repress transcription in Gal4 COS-7 cell reporter assays, as shown in FIG. 6B. Moreover, Grg5 selectively blocked the enhancement of Nkx-dependent repression that is observed in this assay after elevation in the level of Grg4.

Overexpression of Grg5 on neural patterning in vivo was also assessed. Ectopic expression of Grg5 or Grg5^{ΔQ} within the neural tube did not change the expression of Shh by floor plate cells, nor the pattern of expression of the Shh-responsive gene Ptc (FIG. 6C). Thus, the initial response of ventral progenitor cells to Shh signaling appears to be unaffected by overexpression of Grg5.

The influence of Grg5 on the pattern of expression on the complementary class I and class II protein pair, Dbx2 and Nkx6.1 was examined. Ectopic expression of Grg5 resulted in a striking expansion in the domain of Nkx6.1, into the dorsal half of the neural tube (FIG. 6C). The ventral boundary of expression of Dbx2 was also shifted slightly dorsally. In addition, ectopic Dbx2 expression was detected in the dorsal neural tube in a domain overlapping with that of ectopic Nkx6.1 expression (FIG. 6C). No change in the pattern of Nkx6.1 or Dbx2 was detected in the neural tube of embryos in which Gr5^{ΔQ} had been expressed. These results provide evidence that Grg5 overexpression deregulates the pattern of expression of one complementary pair of class I and class II proteins.

Next, the influence of Grg5 on the spatial restriction of expression of a second complementary pair of class I and class II proteins, Pax6 and Nkx2.2 was assessed. Ectopic expression of Grg5 resulted in a dorsal expansion in the domain of expression of Nkx2.2. This result provides evidence that the repression of Nkx2.2 normally imposed by Pax6 depends on Gro/TLE function, despite the fact that Pax6 itself appears to function as a transcriptional activator rather than a repressor (FIG. 4D). No change in the pattern of Nkx2.2 was detected in the neural tube of embryos in which Grg5^{ΔQ} had been misexpressed (FIG. 6C). The ectopic dorsal expression of Nkx2.2 elicited by Grg5, however, did not result in repression in the expression of Pax6. Thus, ectopic dorsal cells that expressed Nkx2.2 coexpressed Pax6, a situation that is not observed when Nkx2.2 itself is misexpressed (FIG. 2C). This finding implies that Nkx2.2 is no longer effective in suppressing Pax6 expression under conditions in which Grg5 is expressed, presumably because the level of Grg/TLE activity is reduced or abolished. Many ectopic dorsal Nkx2.2 expressing cells also expressed Pax6 (boxed area). Overexpression of Grg5 did not affect the expression of Shh nor the Shh responsive gene ptc, Overexpression of Grg5^{ΔQ} did not alter the endogenous expression profile of Nkx6.1, Dbx2, Nkx2.2, Pax6, Shh or Ptc.

The effect of Grg5 overexpression on the Nkx6.1-mediated induction of motor neurons in the dorsal neural tube was analyzed in vivo. In these experiments, shown in FIG. 6D, Nk6.1 was electroporated with either Grg5 or Grg5^{ΔQ} in the neural tube and the ectopic expression of the motor neuron markers Lim3 and HB9 was analyzed. Coelectroporation of Nkx6.1 and Grg5^{ΔQ} resulted in the generation of many Lim3⁺ and HB9⁺ cells within the dorsal half of the neural tube. In contrast, coelectroporation of Nkx6.1 and Grg5, led to the generation of few, if any, ectopic dorsal Lim3⁺ and HB9⁺ cells. Thus, deletion of the TN domain in Nkx6.1 and overexpression of Grg5 both results in an inability of Nkx6.1 to induce ectopic motor neuron generation, supporting the idea that Gro/TLE proteins represent critical cofactors in the neural inductive activity of Nkx6.1.

Together, these results provide strong evidence that inhibition of Gro/TLE activity results in a marked deregulation in the pattern expression of both the complementary class I and class II protein pairs that normally interact to define discrete progenitor domains in the ventral neural tube.

### Example IX

Nkx6.3

A novel Nkx nucleic acid was isolated from Hindbrain mRNA library using 6.3 specific primers. The nucleotide and amino acid sequences are shown in Table 1. The amino acid sequence differs from published sequences at position 127 (S->G, resulting from an a to c change in the nucleic acid) and at position 238 (N->D, resulting from an a to g change in the nucleic acid). The sequences were derived from two different clones isolated from two separate PCRs.

TABLE 1

| | |
|---|---|
| M E S N L Q G T F L L N N T Q L | (SEQ ID NO:13) |
| atg gag tcc aac ctg cag ggg acc ttc ctc ctg aac aac aca cag ctg | (SEQ ID NO:12) |
| A Q F S E M K A P M C Q Y S V Q | |
| gcc cag ttc tca gaa atg aag gct ccc atg tgc cag tac tcg gtg cag | |
| N S F Y K L S P P C L C P Q L A | |
| aac tct ttc tac aag ctc agc cct ccg gga ctt ggc ccc cag ctg gcg | |
| A G T P H G I T D I L S R P V A | |
| gct ggg act ccc cat gga atc act gac atc ctg agc aga ccg gtt gcc | |
| T P N S S L L S G Y P H V A G F | |
| acc ccg aac agc agc ctc ctc tct ggc tac ccc cac gtg gca ggc ttt | |
| G G L S S Q G V Y Y G P Q V G S | |
| ggt gga ctc agc tcc cag ggc gtc tac tat ggc ccc cag gtg ggg agc | |
| F S K A C N E Y P T R T R N C W | |
| ttt tcc aag gct gga aat gag tac ccg acc cgg acc cgg aac tgc tgg | |
| A D T C Q D W H G S A F P C G N | |
| gca gac aca ggc cag gac tgg aga ggc agc gct cgg cca tgc ggc aac | |
| T P D P L S D T I H K K K H T H | |
| act cca gac ccc ctg agt gac act atc cac aag aaa aag cac acc cgc | |
| P T F T G H Q I F A L H K T F E | |
| cct acc ttc acg ggc cac cag atc ttt gcc ctg gag aaa acc ttt gag | |
| Q T K Y L A C P E H A H L A Y S | |
| cag acc aag tac ttg gct ggc cca gag cgg gcg agg ctg gct tat tcc | |
| L G M T E S Q V K V W F Q N R H | |
| ctg ggc atg acg gag tca cag gtc aag gtg tgg ttc cag aac cgc cgg | |
| T K W R K K S A L E P S S S T P | |
| acc aag tgg agg aag aag agc gcc ctg gag ccc tca tcc tcc aca ccc | |
| R A P G G A S C D R A A S E N E | |
| cgg gcc ccg gga ggt gcg agc ggg gac cgc gcg gcc tcg gag aac gag | |
| D D E Y N K P L D P D S D D H K | |
| gac gat gag tac aac aag ccg ctg gac ccg gac tcg gac gac gag aag | |
| I R L L L H K H R A A F S V L S | |
| atc cgc ctg ctg ctg cgc aag cac cgg gcc gcc ttc tcc gtg ctc agc | |
| L C A H S V | |
| ctg ggc gcg cac agc gtg tga | |

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

References

Arber, S., Han, B., Mendelsohn, M., Smith, M., Jessell, T. M., and Sockanathan, S. (1999). Requirement for the homeobox gene Hb9 in the consolidation of motor neuron identity. Neuron 23, 659-674.

Beatus, P., Lundkvist, J., Öberg, C. and Lendahl, U. (1999) The Notch 3 intracellular domain represses Notch 1-mediated activation through Hairy/Enhancer of split (HES) promoters. Development 126, 3925-3935.

Bohinski, R. J., Di Lauro, R., and Whitsett, J. A. (1994). The lung-specific surfactant protein B gene promoter is a target for thyroid transcription factor 1 and hepatocyte nuclear factor 3, indicating common factors for organ-specific gene expression along the foregut axis Mol. Cell. Biol. 14, 5671-5681.

Briscoe, J., Sussel, L., Serup, P., Hartigan-O'Connor, D., Jessell, T. M., Rubenstein, J. L., and Ericson, J. (1999). Homeobox gene Nkx2.2 and specification of neuronal identity by graded Sonic hedgehog signalling. Nature 398, 622-622.

Chiang, C., Litingtung, Y., Lee, E., Young, K. E., Corden, J. L., Westphal, H., and Beachy, P. A. (1996). Cyclopia and defective axial patterning in mice lacking Sonic hedgehog gene function. Nature 383, 407-413.

Choi, C. Y., Kim, Y. H., Kwon, H. J., and Kim, Y. (1999a). The homeodomain protein NK-3 recruits Groucho and a histone deacetylase complex to repress transcription. J. Biol. Chem. 274, 33194-33197.

Choi, C. Y., Lee, Y. M., Kim, Y. H., Park, T., Jeon, B. H, Schultz, R. A., and Kim, Y. (1999b). The homeodomain transcription factor NK-4 acts as either a transcriptional activator or repressor and interacts with the p300 coactivator and the Groucho corepressor. J. Biol. Chem. 274, 31534-31552.

Civitareale, D., Lonigro, R., Sinclair, A. J., and Di Lauro, R. (1989). A thyroid-specific nuclear protein essential for tissue-specific expression of the thyroglobulin promoter.EMBO J. 8, 2537-42.

Damante, E., Pellizzari, L., Esposito, G., Fogolari, F., Viglino, P., Fabbro, D., Tell, G., Formisano, S., and Di Lauro, R. (1996) A molecular code dictates sequence-specific DNA recognition by homeodomains. EMBO J. 15, 4992-5000

Ding, Q., Motoyama, J., Gasca, S., Mo, R., Sasaki, H., Rossant, J., and Hui, C. C. (1998). Diminished Sonic hedgehog signaling and lack of floor plate differentiation in Gli2 mutant mice. Development 125, 2533-2543.

Ericson, J., Morton, S., Kawakami, A., Roelink, H., and Jessell, T. M. (1996). Two critical periods of Sonic Hedgehog signaling required for the specification of motor neuron identity. Cell 87, 661-673.

Ericson, J., Rashbass, P., Schedl, A., Brenner-Morton, S., Kawakami, A., van Heyningen, V., Jessell, T. M., and Briscoe, J. (1997a). Pax6 controls progenitor cell identity and neuronal fate in response to graded Shh signaling. Cell 90, 169-180.

Ericson, J., Briscoe, J., Rashbass, P., van Heyningen, V., and Jessell, T. M. (1997b). Graded sonic hedgehog signaling and the specification of cell fate in the ventral neural tube. Cold Spring Harb Symp Quant Biol 62, 451-466.

Epstein, J., Cai, J., Glaser, T., Jepeal, L., and Maas, R. (1994). Identification of a Pax paired domain recognition sequence and evidence for DNA-dependent conformational changes. J Biol Chem 269, 8355-8361.

Goulding M. D., Lumsden A., and Gruss P. (1993). Signals from the notochord and floor plate regulate the region-specific expression of two Pax genes in the developing spinal cord. Development 117, 1001-1016.

Hamburger, V., and Hamilton, H. L. (1951). A series of normal stages in the development of the chick embryo. J.Morphol. 88, 49-92.

Hartley, D. A., Preiss, A., and Artavanis-Tsakonas, S. (1988). A deduced gene product from the Drosophila neurogenic locus, enhancer of split, shows homology to mammalian G-protein beta subunit. Cell 55, 785-795.

Harvey, R. P (1996) Nk-2 homeobox genes and heart development. Development 126, 203-216.

Hughes, S. H., Greenhouse, J. J., Petropoulos, C. J., and Sutrave, P. (1987). Adaptor plasmids simplify the insertion of foreign DNA into helper-independent retroviral vectors. J Virol 61, 3004-3012.

Jaynes, J. B., and O'Farrel, P. H. (1991). Active repression of transcription by the engrailed homeodomain protein. EMBO J. 10, 1427-1433.

Jensen, J., Serup, P., Karlsen, C., Nielsen, T. F., and Madsen, O. D. (1996). mRNA profiling of rat islet tumors reveals nkx 6.1 as a beta-cell-specific homeodomain transcription factor. J Biol Chem 27, 18749-18758.

Jimenez, G., Paroush, Z., and Ish-Horowics, D. (1997). Groucho acts as a corepressor for a subset of negative regulators, including Hairy and Engrailed. Genes Dev 11, 3072-3082.

Jimenez, G., Verrijzer, C.P., and Ish-Horowics, D. (1999). A conserved motif in goosecoid mediates Groucho-dependent repression in Drosophila embryos. Mol. Cell. Biol. 19, 2080-2087.

John, A., Smith, S. T., and Jaynes, J. B. (1995) Inserting the Ftz homeodomain into Engrailed creates a dominant transcriptional repressor that specifically turns off target genes in vivo. Development 121, 1801-1813.

Jorgensen, M. C., Vesterg\dot{a}rd-Petersen, H., Ericson, J., Madsen, O. D., and Serup, P. (1999). Cloning and DNA-binding properties of the rat pancreatic 1-cell-specific factor Nkx6.1. FEBS letters 461, 287-294.

Kang, T., Martins, T. and Sadowski, I. (1993) Wild type GAL4 binds cooperatively to the GALI-10 UASG in vitro. J. Biol. Chem. 268, 9629-9635.

Koop, K. E., MacDonald, L. M., and Lobe, C. G. (1996). Transcripts of Grg4, a murine groucho-related gene, are detected in adjacent tissues to other murine neurogenic gene homologues during embryonic development. Mech Dev 59, 73-87.

Komuro, I., Schalling, M., Jahn, L., Bodmer, R., Jenkins, N. A., Copeland, N. G. and Izumo, S. (1993). Gtx: A novel murine homeobox-containing gene, expressed specifically in glial cells of the brain and germ cells of testis,has a transcriptional repressor activity in vitro for a serum-inducible promoter. EMBO J. 12, 1387-1401.

Kozmik, Z., Czerny, T., and Busslinger, M. (1997). Alternatively spliced insertions in the paired domain restrict the DNA sequence specificity of Pax6 and Pax8. EMBO J 16, 6793-6803.

Leber, S. M., and Sanes, J. R. (1995). Migratory paths of neurons and glia in the embryonic chick spinal cord. J Neurosci 15, 1236-1248.

Leon, C., and Lobe, C. G. (1997). Grg3, a murine Groucho-related gene, is expressed in the developing nervous system and in mesenchyme-induced epithelial structures. Dev Dyn 208, 11-24.

Lints, T. J., Parsons, L. M., HArtley, L., Lyons, I. and Harvey, R. P. (1993) Nkx-2.5: a novel murine homeobox gene expressed in early heart progenitor cells and their myogenic descendants. Development 119, 419-431.

Logan, C., Hanks, M. C., Noble-Topham, S., Nallainathan, D., Provart, N. J., and Joyner, A. L. (1992). Cloning and sequence comparision of the mouse, human, and chicken engrailed genes reveal potential functional domains and regulatory regions. Devel. Genetics 13, 345-58.

Ma, Q., Sommer, L., Cserjesi, P., and Anderson, D. J. (1997). MashI and neurogenin1 expression patterns define complementary domains of neuroepithelium in the developing CNS and are correlated with regions expressing notch ligands. J Neurosci 17, 3644-3652.

Mallo, M., Franco del Amo, F., and Gridley, T. (1993). Cloning and developmental expression of Grg, a mouse gene related to the groucho transcript of the Drosophila Enhancer of split complex. Mech Dev 42, 67-76.

Mansouri, A., and Gruss, P. (1998). Pax3 and Pax7 are expressed in commissural neurons and restrict ventral neuronal identity in the spinal cord. Mech Dev 78, 171-178.

Marigo, V., and Tabin, C.J. (1996). Regulation of patched by sonic hedgehog in the developing neural tube. Proc Natl Acad Sci U S A. 93, 9346-9351.

Marti, E., Takada, R., Bumcrot, D. A., Sasaki, H., and McMahon, A. P. (1995). Distribution of Sonic hedgehog peptides in the developing chick and mouse embryo. Development 121, 2537-2547.

Matise, M. P., Epstein, D. J., Park, H. L., Platt, K. A., and Joyner, A. L. (1998). Gli2 is required for induction of floor plate and adjacent cells, but not most ventral neurons in the mouse central nervous system. Development 125, 2759-2770.

Miyasaka, H., Choudhury, B. K., Hou, E. W. and Li, S. S. (1993). Molecular cloning and expression of mouse and human cDNA encoding AES and ESG proteins with strong similarity to Drosophila enhancer of split groucho protein. Eur. J. Biochem. 216, 343-352.

Morgan, B. A., and Fekete, D. M. (1996). Manipulating gene expression with replication-competent retroviruses. Methods Cell Biol 51, 185-218.

Muramatsu, T., Mizutani, Y., Ohmori, Y., and Okumura, J. (1997). Comparison of three nonviral transfection methods for foreign gene expression in early chicken embryos in ovo. Biochem Biophys Res Commun 230, 376-380.

Pabst, O., Herbrand, H., and Arnold, H. H. (1998). Nkx2.9 is a novel homeobox transcription factor which demarcates ventral domains in the developing mouse CNS. Mech Dev 73, 85-93.

Paroush, Z., Finley Jr, R., Kidd, T., Wainwright, S. M., Ingham, P. W., Brent, R., and Ish-Horowicz, D. (1994) Groucho is required for Drosophila neurogenesis, segmentation, and sex determination and interacts directly with hairy-related bHLH proteins. Cell 79, 805-815.

Perlmann, T., and Jansson, L. (1995) A novel pathway for vitamin A signaling mediated by RXR heterodimerization with NGFI-B and Nurrl. Genes Dev 9, 769-782.

Pierani , A., Brenner-Morton, S., Chiang, C., and Jessell, T. M. (1999). A sonic hedgehog-independent, retinoid-activated pathway of neurogenesis in the ventral spinal cord. Cell 97 903-915.

Pierani, A., Moran-Rivard, L., Sunshine, M. J., Littman, D. R., Goulding, M., and Jessell, T. M. (2001) Control of interneuron fate in the developing spinal cord by the progenitor homeodomain protein Dbxl. Neuron, 29, 367-384.

Pfaff, S. L., Mendelsohn, M., Stewart, C. L., Edlund, T., and Jessell, T. M.(1996). Requirement for LIM homeobox gene Isl I in motor neuron generation reveals a motor neuron-dependent step in interneuron differentiation. Cell 84, 309-320.

Qiu, M., Shimamura, K., Sussel, L., Chen, S., and Rubenstein, J. L. (1998). Control of anteroposterior and dorsoventral domains of Nkx-6.1 gene expression relative to other Nkx genes during vertebrate CNS development. Mech Dev 72, 77-88.

Rangini, Z., Ben-Yehuda, A., Shapira, E., Gruenbaum, Y., and Fainsod, A. (1991). CHox E, a chicken homeogene of the H2.0 type exhibits dorso-ventral restriction in the proliferating region of the spinal cord. Mech Dev 35, 13-24.

Ren, B., Chee, K. J., Kim, T. H. and Maniatis, T. (1999). PRDI-BF1/Blimp1 repression is mediated by corepressors of the Groucho family of proteins. Genes Dev. 13, 125-137.

Roose, J., Molenaar, M., J., Hurenkamp, J., Brantjes, H., Moerer, P., van de Wetering, M., Destree, O., and Clevers, H. (1998). The Xenopus Wnt effector XTcf-3 interacts with Groucho-related transcriptional repressors. Nature 395, 608-612.

Roelink, H., Augsburger, A., Heemskerk, J., Korzh, V., Norlin, S., Ruiz i Altaba, A., Tanabe, Y., Placzek, M., Edlund, T., Jessell, T. M., and Dodd, J. (1994). Floor plate and motor neuron induction by vhh-1, a vertebrate homologue of hedgehog expressed by the notochord. Cell 76, 761-775.

Roelink, H., Porter, J. A., Chiang, C., Tanabe, Y., Chang, D. T., Beachy, P. A., and Jessell, T. M. (1995). Floor plate and motor neuron induction by different concentrations of the amino-terminal cleavage product of sonic hedgehog autoproteolysis. Cell 81, 445-455.

Sander, M., Paydar, S., Ericson, J., Briscoe, J., Berber, E., German, M., Jessell T. M., and Rubenstein J. L. (2000) Ventral neural patterning by Nkx homeobox genes: Nkx6.1 controls somatic motor neuron and ventral interneuron fates. Genes Dev. 17, 2134-9.

Schaeren-Wiemers, N., and Gerfin-Moser, A. (1993). A single protocol to detect transcripts of various types and expression levels in neural tissue and cultured cells: in situ hybridization using digoxigenin-labelled cRNA probes. Histochemistry 100, 431-440.

Sharma, K., Sheng, H. Z., Lettieri, K., Li, H., Karavanov, A., Potter, S., Westphal, H., and Pfaff, S. L. (1998). LIM homeodomain factors Lhx3 and Lhx4 assign subtype identities for motor neurons. Cell 95, 817-828.

Smith, S. T., and Jaynes, J. B. (1996). A conserved region of engrailed, shared among all en-, gsc-. NK1, Nk2-, and msh-class homeoproteins, mediates active transcriptional repression in vivo. Development 122, 3141-3150.

Sommer, L., Ma, Q., and Anderson, D. J. (1996). Neurogenins, a novel family of atonal-related bHLH transcription factors, are putative mammalian neuronal determination genes that reveal progenitor cell heterogeneity in the developing CNS and PNS. Mol Cell Neurosci 8, 221-241.

Struhl, G., Johnston, P., and Lawrence, P. A. (1992). Control of Drosophila body pattern by the hunchback morphogen gradient. Cell 69, 237-249.

Sussel, L., Marin, O., Kimura, S., and Rubenstein, J. L. (1999) Loss of Nkx2.1 homeobox gene function results in a ventral to dorsal molecular respecification within the basal telencephalon: evidence for a transformation of the pallidum into the striatum. Development 126,3359-3370.

Thaler, J., Harrison, K., Sharma, K., Lettieri, K., Kehrl, J., and Pfaff S. L. (1999). Active suppression of interneuron programs within developing motor neurons revealed by analysis of homeodomain factor HB9. Neuron 23, 675-687.

Tanabe, Y., William, C., and Jessell, T. M. (1998). Specification of motor neuron identity by the MNR2 homeodomain protein. Cell 95, 67-80.

Watada, H,, Mirmira, R. G., Kalamaras, J., and German, M. S. (2000). Intramolecular control of transcriptional activity by the NK2-specific domain in NK-2 homeodomain proteins. Proc. Natl. Acad. Sci. USA 97, 9443-9448.

White, J., Brou, C., Wu, J., Lutz, Y., Moncollin, V., and Chambon, P. (1992). The acidic transcriptional activator GAL-VP16 acts on preformed template-committed complexes. EMBO J. 11, 2229-2240.

1. A method of guiding the fate of differentiation of a cell into a specific cell type, comprising
a) providing a sample comprising the cell;
b) contacting the sample with a Groucho-interacting protein (GIP) in an amount and for a time sufficient to result in the formation of a complex between the GIP and a Groucho corepressor protein;
wherein the GIP and Groucho-corepressor protein complex represses DNA transcription and suppresses alternative pathways of differentiation, thereby guiding the fate of differentiation of the cell into a specific cell type.

2. The method of claim 1 wherein the method further comprises the step of contacting the cell with an exogenous Groucho corepressor protein.

3. The method of claim 1 wherein the Groucho corepressor protein is endogenous to the cell.

4. The method of claim 1, wherein the Groucho corepressor protein is selected from the group consisting of Grg1, Grg2, Grg3, and Grg4 and their human homologs.

5. The method of claim 1, wherein the GIP comprises a TN-like domain.

6. The method of claim 1, wherein the GIP is a homeodomain polypeptide.

7. The method of claim 6 wherein the homeodomain polypeptide is a class II homeodomain polypeptide.

8. The method of claim 7 wherein the class II homeodomain polypeptide is a member of the Nkx polypeptide family.

9. The method of claim 8, wherein the Nkx polypeptide is selected from the group consisting of Nkx2.2, Nkx2.9, Nkx6.1, Nkx6.2, and Nkx6.3 and their human homologs.

10. The method of claim 9, wherein the guided differentiation results in the cell being differentiated into a motor neuron cell.

11. The method of claim 6 wherein the homeodomain polypeptide is a class I homeodomain polypeptide.

12. The method of claim 11 wherein the class I homeodomain polypeptide is selected from the group consisting of members of the Pax, Dbx, and Irx polypeptide families.

13. The method of claim 1 wherein the cell is a stem cell.

14. The method of claim 13 wherein the cell is a neural stem cell.

15. The method of claim 1 wherein the cell is a progenitor cell.

16. The method of claim 1, wherein the specific cell type into which the cell differentiates is a neuron.

17. The method of claim 16, wherein the neuron is an intemeuron, a motor neuron or a projection neuron.

18. The method of claim 17, wherein the projection neuron is selected from the group consisting of a dopaminergic neuron, a cortical neuron, a gaba-ergic neuron and a glutaminergic neuron.

19. The method of claim 1, wherein the specific cell type into which the cell differentiates is selected from the group consisting of a stem cell, a cell of the peripheral nervous system, a kidney cell, a heart muscle cell, a pancreatic cell, a skin cell, a liver cell, and a white or red blood cell.

20. The method of claim 1, wherein the GIP is selected from the group consisting of Nkx6.1, Nkx6.2, Nkx6.3, and Nkx2.2 and the cell type into which the cell differentiates is a beta cell producing insulin.

21. The method of claim 1, wherein the contacting of the sample with a GIP occurs either in vitro, ex vivo, or in vivo.

22. The method of claim 21, wherein the contacting of the sample with a GIP occurs either ex vivo.

23. The method of claim 1, wherein the GIP is a polypeptide comprising an amino acid sequence selected from the group consisting of SEQ ID NO: 7 and 13.

24. The method of claim 1, wherein the GIP is a polypeptide comprising the amino acid sequence X_{AA1}-X_{AA2}-X_{AA3}-X_{AA4}-X_{AA5}-X_{AA6}-X_{AA7}-X_{AA8}-X_{AA9}-X_{AA10}-X_{AA11}, wherein X_{AA1} is Thr, Leu, or Ser; X_{AA2} is Gly or Pro; X_{AA3} is Phe or His; X_{AA4} is Ser, Thr, Gly, or His; X_{AA5} is Val or Ile; X_{AA6} is Lys, Arg, Asn, or Ser; X_{AA7} is Asp or Ser; X_{AA8} is Is1 or Leu; X_{AA9} is Leu; X_{AA10} is Asp, Asn, Ser, or Gly; and X_{AA11} is Leu or Arg.

25. An isolated polypeptide comprising an amino acid sequence selected from the group consisting of:
(a) an amino acid sequence of SEQ ID NO: 7 or 13; and
(b) a variant of an amino acid sequence of SEQ ID NO: 7 or 13, wherein one or more amino acid residues in said variant differs from the amino acid sequence of said mature form, provided that said variant differs in no more than 15% of amino acid residues from said amino acid sequence.

26. The polypeptide of claim 25, wherein said polypeptide comprises the amino acid sequence of a naturally-occurring allelic variant of an amino acid sequence of SEQ ID NO: 7 or 13.

27. The polypeptide of claim 26, wherein said allelic variant comprises an amino acid sequence that is the translation of a nucleic acid sequence differing by a single nucleotide from the nucleic acid sequence of SEQ ID NOS: 12.

28. The polypeptide of claim 25, wherein the amino acid sequence of said variant comprises a conservative amino acid substitution.

29. An isolated nucleic acid molecule comprising a nucleic acid sequence encoding a polypeptide comprising an amino acid sequence,
wherein the amino acid sequence encoded by the nucleic acid molecule is selected from the group consisting of:
(a) the amino acid sequence of SEQ ID NO: 7 or 13;
(b) a variant of an amino acid sequence of SEQ ID NO: 7 or 13, wherein one or more amino acid residues in said variant differs from the amino acid sequence of said mature form, provided that said variant differs in no more than 15% of amino acid residues from said amino acid sequence; and
wherein the nucleic acid molecule is selected from the group consisting of:
(c) a nucleic acid fragment encoding at least a portion of a polypeptide comprising an amino acid sequence of SEQ ID NO: 7 or 13, or a variant of said polypeptide, wherein one or more amino acid residues in said variant differs from the amino acid sequence of said mature form, provided that said variant differs in no more than 15% of amino acid residues from said amino acid sequence; and
(d) a nucleic acid molecule comprising the complement of (a), (b), or (c).

30. The nucleic acid molecule of claim 29, wherein the nucleic acid molecule comprises the nucleotide sequence of a naturally-occurring allelic nucleic acid variant.

31. The nucleic acid molecule of claim 29, wherein the nucleic acid molecule encodes a polypeptide comprising the amino acid sequence of a naturally-occurring polypeptide variant.

32. The nucleic acid molecule of claim 29, wherein the nucleic acid molecule differs by a single nucleotide from the nucleic acid of SEQ ID NO: 12.

33. The nucleic acid molecule of claim 29, wherein said nucleic acid molecule comprises a nucleotide sequence selected from the group consisting of:
(a) the nucleotide sequence of SEQ ID NO: 12;
(b) a nucleotide sequence differing by one or more nucleotides from the nucleotide sequence of SEQ ID NO: 12, provided that no more than 20% of the nucleotides differ from said nucleotide sequence;
(c) a nucleic acid fragment of (a); and
(d) a nucleic acid fragment of (b).

34. The nucleic acid molecule of claim 29, wherein said nucleic acid molecule hybridizes under stringent conditions to the nucleotide sequence of SEQ ID NOS: 12, or a complement of said nucleotide sequence.

35. The nucleic acid molecule of claim 29, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

36. A vector comprising the nucleic acid molecule of claim 35.

37. The vector of claim 36, further comprising a promoter operably-linked to said nucleic acid molecule.

38. A cell comprising the vector of claim 36.

39. An antibody that binds immunospecifically to the polypeptide of claim 25.

40. The antibody of claim 39, wherein said antibody is a monoclonal antibody.

41. The antibody of claim 39, wherein the antibody is a humanized antibody.

42. A peptide less than 400 amino acids in length that includes the amino acid sequence X_{AA1}-X_{AA2}-X_{AA3}-X_{AA4}-X_{AA5}-X_{AA6}-X_{AA7}-X_{AA8}-X_{AA9}-X_{AA10}-X_{AA11}, wherein X_{AA1} is Thr, Leu, or Ser; X_{AA2} is Gly or Pro; X_{AA3} is Phe or His; X_{AA4} is Ser. Thr, Gly, or His; X_{AA5} is Val or Ile; X_{AA6} is Lys, Arg, Asn, or Ser; X_{AA7} is Asp or Ser; X_{AA8} is Ile or Leu; X_{AA9} is Leu; X_{AA10} is Asp, Asn, Ser, or Gly; and X_{AA9} is Leu or Arg.

43. The peptide of claim 42, wherein said peptide is less than 100 amino acids in length.

44. The peptide of claim 42, wherein said peptide comprises the amino acid sequence of SEQ ID NO: 7.

45. A purified complex comprising a first polypeptide and a second polypeptide, wherein the first polypeptide comprises a GIP and the second protein comprises a Groucho corepressor protein.

46. The complex of claim 45, wherein the first polypeptide is labeled.

47. The complex of claim 45, wherein the second polypeptide is labeled.

48. The complex of claim 47, wherein the first polypeptide is labeled.

49. The complex of claim 45, wherein the first polypeptide is selected from the group consisting of class I and class II homeodomain polypeptides.

50. The complex of claim 45, wherein the first polypeptide contains a TN domain.

51. The complex of claim 45, wherein the Groucho corepressor protein is selected from the group consisting of Grg1, Grg2, Grg3, or Grg4 and their mammalian homologs.

52. The complex of claim 45, wherein the GIP comprises a TN-like domain.

53. The complex of claim 45, wherein the GIP is a homeodomain polypeptide.

54. A purified complex comprising a first polypeptide and a second polypeptide, wherein said first polypeptide comprises a region of amino acids of a GIP sufficient to allow the first polypeptide to bind the second polypeptide, and wherein the second polypeptide comprises a region of amino acids of a Groucho corepressor protein sufficient to bind the first polypeptide.

55. A chimeric polypeptide comprising a first domain covalently linked to a second domain, wherein the first domain comprises six or more amino acids of the first polypeptide of claim 45 and the second domain comprises six or more amino acids of the second polypeptide of claim 45.

56. The chimeric polypeptide of claim 55 wherein the first domain comprises a GIP binding domain and the second domain comprises a groucho corepressor binding domain.

57. The chimeric polypeptide of claim 28, wherein the first domain comprises a TN domain.

58. A nucleic acid encoding the chimeric polypeptide of claim 55.

59. A vector comprising the nucleic acid of claim 58.

60. A cell comprising the vector of claim 58.

61. An antibody which specifically binds the complex of claim 45.

62. A kit comprising a reagent which can specifically detect the complex of claim 45.

63. The kit of claim 62, wherein the reagent is selected from the group consisting of an antibody specific for the complex, an antibody specific for the first polypeptide, and an antibody specific for the second polypeptide.

64. A method of identifying an agent which modulates the stability or activity of the complex of claim 45 comprising:
a) providing the complex:
b) contacting the complex with a test agent; and
c) detecting whether the test agent modulates the stability or activity of the complex.

65. A method of identifying an agent which disrupts a polypeptide complex, the method comprising
a) providing the complex of claim 45;
b) contacting the complex with a test agent; and
c) detecting the presence of a polypeptide displaced from the complex,
wherein the presence of displaced polypeptide indicates the agent disrupts the complex.

66. A method for the screening of a candidate substance interacting with the complex of claim 45, comprising:
a) providing the complex of claim 45;
b) obtaining a candidate substance;
c) bringing into contact the complex with the candidate substance; and
d) detecting the complexes formed between the polypeptide and the candidate substance.

67. A method for the screening of a candidate substance interacting with a Groucho-corepressor protein, comprising
a) providing a Groucho-corepressor protein;
b) obtaining a candidate substance;
c) bringing into contact the polypeptide with the candidate substance; and
d) detecting the complexes formed between the polypeptide and the candidate substance.

68. A method for inhibiting the guided differentiation of a cell resulting in the impairment of ventral patterning, the method comprising:
contacting the complex of claim 45 with an agent that disrupts the complex, thereby inhibiting the guided differentiation by disrupting the complex, which is necessary for differentiation.

69. A method of identifying a polypeptide complex in a subject, the method comprising:
a) providing a biological sample from a subject; and
b) detecting, if present, the polypeptide complex of claim 45 in the sample, thereby identifying the complex.

70. A method of determining altered expression of a polypeptide in a subject, the method comprising
a) providing a biological sample from the subject;
b) measuring the level of the complex of claim 45 in the sample; and
c) comparing the level of the complex from step b) to the level of the complex in a reference sample whose level of the complex of claim 45 is known;
thereby determining whether the subject has altered expression of the polypeptide.

71. A method of treating or preventing a disease or disorder involving altered levels of the complex of claim 45 by administering a therapeutically effective amount of at least one molecule that modulates the function of the complex to a subject in need thereof.
